(19) United States
(12) Patent Application Publication
(10) Pub. No.: US 2024/0206757 A1
(43) Pub. Date: Jun. 27, 2024

(54) ELECTROSTATIC CAPACITY SENSOR AND MEASURING INSTRUMENT

(71) Applicant: Murata Manufacturing Co., Ltd., Kyoto (JP)

(72) Inventors: Yoshie KURAMOCHI, Kyoto (JP); Jun TAKAGI, Kyoto (JP); Tomoki TAKAHASHI, Kyoto (JP)

(21) Appl. No.: 18/598,121

(22) Filed: Mar. 7, 2024

Related U.S. Application Data
(63) Continuation of application No. PCT/JP2022/029528, filed on Aug. 1, 2022.

(30) Foreign Application Priority Data
Sep. 10, 2021 (JP) 2021-148131
May 31, 2022 (JP) 2022-088969

(51) Int. Cl. A61B 5/0537; A61B 5/00

(52) U.S. Cl. CPC A61B 5/0537; A61B 5/4875

(57) ABSTRACT

There are provided an electrostatic capacity sensor and a measuring instrument that can reduce influence of a stray capacitance on detection of an electrostatic capacity. An electrostatic capacity sensor includes a sensor unit having a first electrode and a second electrode constituting a capacitor, and an electrostatic capacity detection circuit that is connected to the sensor unit. The electrostatic capacity detection circuit includes a charge and discharge circuit that is connected to the first electrode and the second electrode to charge and discharge the capacitor, a control circuit that controls the charge and discharge circuit such that the capacitor repeats charge and discharge, and an auxiliary capacity circuit that has at least one of a first auxiliary capacitor that is connected to the first electrode in parallel with the capacitor and a second auxiliary capacitor that is connected to the second electrode in parallel with the capacitor.

### CROSS REFERENCE TO RELATED APPLICATION

This is a continuation of International Application No. PCT/JP2022/029528 filed on Aug. 1, 2022 which claims priority from Japanese Patent Application No. 2021-148131 filed on Sep. 10, 2021 and Japanese Patent Application No. 2022-088969 filed on May 31, 2022. The contents of these applications are incorporated herein by reference in their entireties.

### BACKGROUND OF THE DISCLOSURE

### Field of the Disclosure

The present disclosure relates to an electrostatic capacity sensor and a measuring instrument.

### Description of the Related Art

Patent Document 1 discloses an intraoral moisture measuring instrument as a measuring instrument including an electrostatic capacity type sensor (electrostatic capacity sensor). The intraoral moisture measuring instrument described in Patent Document 1 includes a swinging member that swings about a predetermined swinging center with respect to a main body, a moisture content detection unit that is provided on a distal end side of the swinging member, and directly or indirectly abuts on a measurement portion within an oral cavity to detect a moisture content, and a biasing member that biases the swinging member in one of swinging directions. The moisture content detection unit includes an electrostatic capacity type sensor.
Patent Document 1: International Publication No. WO 2015/125222

### BRIEF SUMMARY OF THE DISCLOSURE

In Patent Document 1, the intraoral moisture measuring instrument is used in a state of being held by person with hand. Thus, the intraoral moisture measuring instrument is influenced by the stray capacitance generated between the human body and the intraoral moisture measuring instrument, and measurement accuracy may decrease.

The present disclosure provides an electrostatic capacity sensor and a measuring instrument that can reduce influence of a stray capacitance on detection of an electrostatic capacity.

An electrostatic capacity sensor according to an aspect of the present disclosure includes a sensor unit that has a first electrode and a second electrode constituting a capacitor, and an electrostatic capacity detection circuit that is connected to the sensor unit. The electrostatic capacity detection circuit includes a charge and discharge circuit that is connected to the first electrode and the second electrode to charge and discharge the capacitor, a control circuit that controls the charge and discharge circuit such that the capacitor repeats charging and discharging, and an auxiliary capacity circuit that has at least one of a first auxiliary capacitor connected to the first electrode in parallel with the capacitor and a second auxiliary capacitor connected to the second electrode in parallel with the capacitor.

A measuring instrument according to another aspect of the present disclosure includes the electrostatic capacity sensor, and a handheld housing that accommodates the electrostatic capacity sensor.

The aspects of the present disclosure can reduce the influence of the stray capacitance on the detection of the electrostatic capacity.

### BRIEF DESCRIPTION OF THE SEVERAL VIEWS OF THE DRAWINGS

FIG. 1 is a schematic diagram of a configuration example of a measuring instrument according to a first embodiment.

FIG. 2 is a circuit diagram of a configuration example of an electrostatic capacity sensor of the measuring instrument of FIG. 1.

FIG. 3 is a schematic cross-sectional view of a configuration example of a sensor unit of the electrostatic capacity sensor of FIG. 2.

FIG. 4 is a schematic plan view of the sensor unit of FIG. 3.

FIG. 5 is a schematic bottom view of the sensor unit of FIG. 3.

FIG. 6 is a timing chart of an example of an operation of an electrostatic capacity detection circuit of the electrostatic capacity sensor of FIG. 2.

FIG. 7 is an explanatory diagram of an example of an operation of the electrostatic capacity detection circuit of FIG. 2.

FIG. 8 is an explanatory diagram of an example of an operation of the electrostatic capacity detection circuit of FIG. 2.

FIG. 9 is an explanatory diagram of an example of an operation of the electrostatic capacity detection circuit of FIG. 2.

FIG. 10 is an explanatory diagram of an example of an operation of the electrostatic capacity detection circuit of FIG. 2.

FIG. 11 is an explanatory diagram of an example of an operation of the electrostatic capacity detection circuit of FIG. 2.

FIG. 12 is an explanatory diagram of an example of an operation of the electrostatic capacity detection circuit of FIG. 2.

FIG. 13 is a timing chart of an example of an operation of an electrostatic capacity detection circuit of a comparative example.

FIG. 14 is an explanatory diagram of a capacitance generated when the measuring instrument of FIG. 1 is used.

FIG. 15 is a timing chart of an example of an operation of an electrostatic capacity detection circuit according to a modification example of the first embodiment.

FIG. 16 is a circuit diagram of a configuration example of an electrostatic capacity sensor of a measuring instrument according to a second embodiment.

FIG. 17 is a timing chart of an example of an operation of an electrostatic capacity detection circuit of the electrostatic capacity sensor of FIG. 16.

FIG. 18 is an explanatory diagram of an example of an operation of the electrostatic capacity detection circuit of FIG. 16.

FIG. 19 is an explanatory diagram of an example of an operation of the electrostatic capacity detection circuit of FIG. 16.

FIG. 20 is an explanatory diagram of an example of an operation of the electrostatic capacity detection circuit of FIG. 16.

FIG. 21 is an explanatory diagram of an example of an operation of the electrostatic capacity detection circuit of FIG. 16.

FIG. 22 is an explanatory diagram of an example of an operation of the electrostatic capacity detection circuit of FIG. 16.

FIG. 23 is an explanatory diagram of an example of an operation of the electrostatic capacity detection circuit of FIG. 16.

FIG. 24 is a timing chart of an example of an operation of an electrostatic capacity detection circuit according to a modification example of the second embodiment.

FIG. 25 is a schematic diagram of a configuration example of a measuring instrument according to a third embodiment.

FIG. 26 is a schematic perspective view of a configuration example of a head portion of the measuring instrument of FIG. 25.

FIG. 27 is a schematic perspective view of a configuration example of a head portion of a measuring instrument according to a fourth embodiment.

FIG. 28 is an explanatory diagram of a configuration example of a sensor unit of an electrostatic capacity sensor of the measuring instrument of FIG. 27.

FIG. 29 is a schematic cross-sectional view of a configuration example of a sensor unit of the electrostatic capacity sensor of the measuring instrument of FIG. 27.

FIG. 30 is a schematic plan view of the sensor unit of FIG. 29.

FIG. 31 is a schematic bottom view of the sensor unit of FIG. 29.

FIG. 32 is a schematic cross-sectional view of a configuration example of a sensor unit of an electrostatic capacity sensor of a measuring instrument according to a fifth embodiment.

FIG. 33 is a schematic plan view of the sensor unit of FIG. 32.

FIG. 34 is a schematic bottom view of the sensor unit of FIG. 32.

FIG. 35 is a schematic cross-sectional view of a configuration example of a sensor unit of an electrostatic capacity sensor of a measuring instrument according to a sixth embodiment.

FIG. 36 is a schematic cross-sectional view of a configuration example of a sensor unit of an electrostatic capacity sensor of a measuring instrument according to a seventh embodiment.

FIG. 37 is a schematic cross-sectional view of a configuration example of a sensor unit of an electrostatic capacity sensor of a measuring instrument according to an eighth embodiment.

FIG. 38 is a schematic perspective view of a configuration example of a head portion of a measuring instrument according to a ninth embodiment.

FIG. 39 is a schematic perspective view of a configuration example of a head portion of a measuring instrument according to a tenth embodiment.

FIG. 40 is a schematic diagram of a configuration example of a measuring instrument according to an eleventh embodiment.

### DETAILED DESCRIPTION OF THE DISCLOSURE

### 1. Embodiment

Hereinafter, embodiments will be described in detail with reference to the drawings as appropriate. However, more detailed description than necessary may be omitted. For example, detailed description of already well-known matters or redundant description of substantially the same configuration may be omitted. This is to avoid the following description from becoming unnecessarily redundant and to facilitate the understanding of those skilled in the art. Note that, the inventor (s) provide the accompanying drawings and the following description in order for those skilled in the art to fully understand the present disclosure, and are not intended to limit the subject matters of the claims by the accompanying drawings and the following description.

Positional relationships, such as up, down, left, and right, are based on the positional relationships illustrated in the drawings unless otherwise specified. Each drawing described in the following embodiments is a schematic drawing, and ratios of a size and a thickness of each component in each drawing does not constantly reflect actual dimensional ratios. In addition, the dimensional ratio of each element is not limited to a ratio illustrated in the drawings.

In the following embodiment, an expression “A and B are connected to C and D, respectively” and similar expressions mean that “A is connected to C and B is connected to D” and does not mean that “A and B are connected to C and A and B are connected to D”. In addition, an expression “a plurality of A's is connected to a plurality of C's, respectively” and similar expressions mean that “A and C are connected in a one-to-one correspondence”.

In a circuit configuration of the present disclosure, an expression “connected” includes not only a case of being directly connected by a connection terminal and/or a wiring conductor, but also a case of being electrically connected with another circuit element interposed therebetween. In addition, an expression “connected between A and B” means that another component is connected to both A and B between A and B.

### 1.1 First Embodiment

### 1.1.1 Configuration

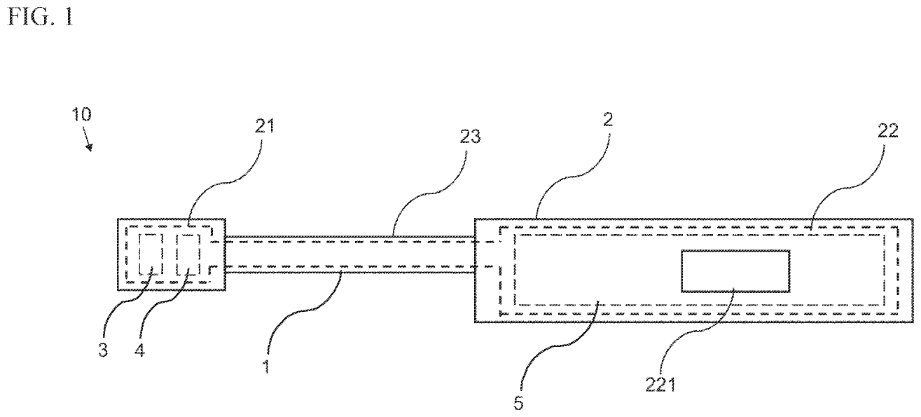

FIG. 1 is a schematic diagram of a configuration example of a measuring instrument 10 according to a first embodiment. The measuring instrument 10 is a moisture measuring instrument for measuring a moisture content of a measurement target. The measurement target is, for example, an organism. In particular, the measurement target is an oral cavity of the organism. In the present embodiment, the measuring instrument 10 is used to measure a moisture content of a specific part of a human oral cavity. In a healthcare application, the measuring instrument 10 is also referred to as an oral moisture meter.

The measuring instrument 10 in FIG. 1 is an electrostatic capacity type moisture measuring instrument. The measuring instrument 10 includes an electrostatic capacity sensor 1 and a handheld housing 2.

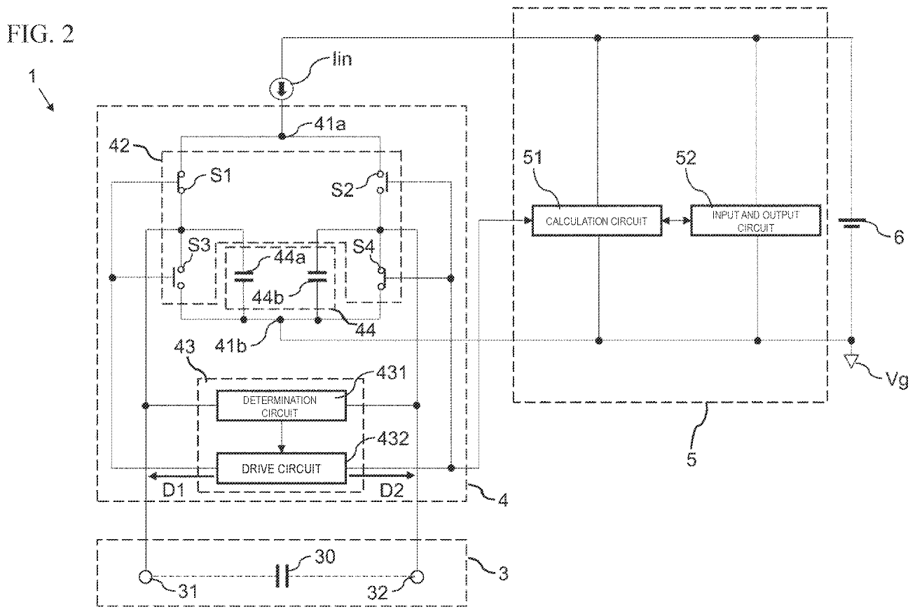

The handheld housing 2 accommodates the electrostatic capacity sensor 1. The handheld housing 2 has a size and a weight that can be held by a person with one hand. The handheld housing 2 has a waterproof structure and protects the electrostatic capacity sensor 1 within the handheld housing 2 from moisture. The handheld housing 2 in FIG. 1 has a rod shape. The handheld housing 2 includes a head portion 21, a grip portion 22, and a probe portion 23. The handheld housing 2 in FIG. 1 has a shape like a so-called toothbrush. The head portion 21 is a part of the handheld housing 2 that comes into contact with the measurement target. The head portion 21 is disposed at a first end of the handheld housing 2 (left end in FIG. 1). In the present embodiment, the head portion 21 is placed in the human oral cavity when used. The grip portion 22 is a portion of the handheld housing 2 that is gripped with the hand. The grip portion 22 is disposed at a second end of the handheld housing 2 (right end in FIG. 1). The grip portion 22 includes a conductive portion 221. The conductive portion 221 is exposed on a surface of the grip portion 22. The conductive portion 221 may be at a position that comes into contact with the hand of the person when the person grips the grip portion 22. The conductive portion 221 is connected to a reference potential Vg (see FIG. 2) to be described later. The probe portion 23 couples the head portion 21 and the grip portion 22. A length of the probe portion 23 may be set such that the person can easily grip the grip portion 22 and bring the head portion 21 into contact with the measurement target.

The electrostatic capacity sensor 1 obtains the moisture content of the measurement target based on the electrostatic capacity. FIG. 2 is a circuit diagram of a configuration example of the electrostatic capacity sensor 1. The electrostatic capacity sensor 1 in FIG. 2 includes a sensor unit 3, an electrostatic capacity detection circuit 4, and a processing circuit 5. In the present embodiment, the sensor unit 3 and the electrostatic capacity detection circuit 4 are positioned in the head portion 21 of the handheld housing 2. The electrostatic capacity detection circuit 4 may be positioned in the probe portion 23 of the handheld housing 2. In the present embodiment, the processing circuit 5 is positioned in the grip portion 22 of the handheld housing 2. As illustrated in FIG. 2, the electrostatic capacity sensor 1 obtains power necessary for an operation of the electrostatic capacity sensor 1 from a DC power supply 6. The DC power supply 6 may be a primary battery or a secondary battery. The DC power supply 6 may be replaceable.

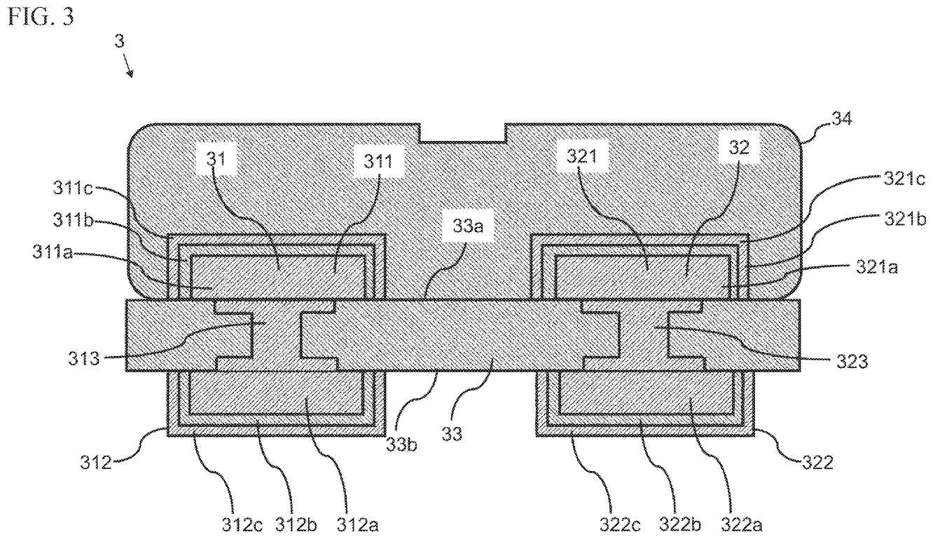

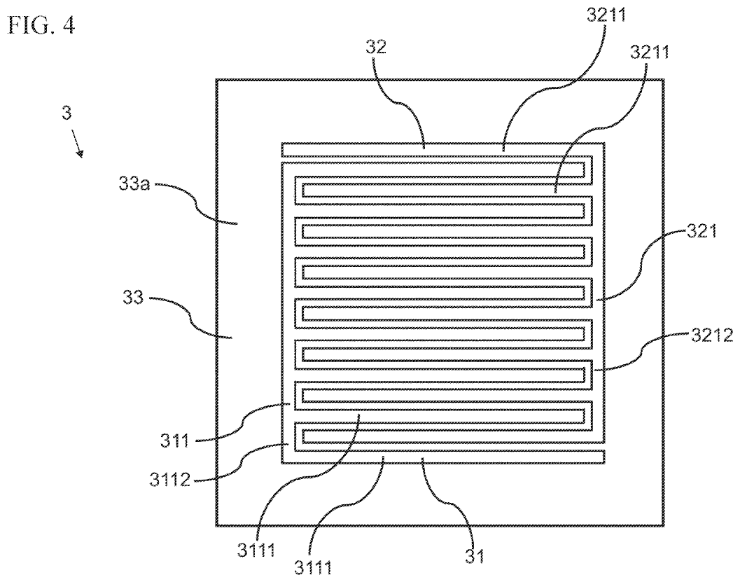

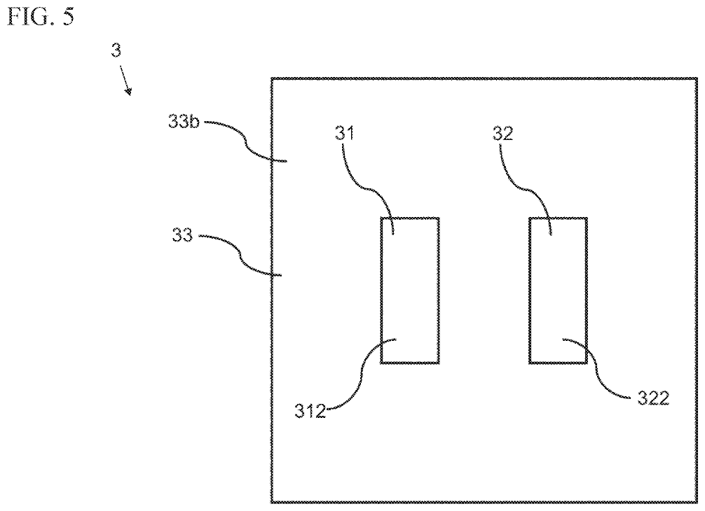

The sensor unit 3 in FIG. 2 includes a first electrode 31 and a second electrode 32. The sensor unit 3 is formed such that the first and second electrodes 31 and 32 form a capacitor 30 together with a part of the measurement target by bringing the first and second electrodes 31 and 32 into contact with the measurement target. Hereinafter, a configuration of the sensor unit 3 will be described in further detail with reference to FIGS. 3 to 5. FIG. 3 is a schematic cross-sectional view of a configuration example of the sensor unit 3. FIG. 4 is a schematic plan view of the sensor unit 3. FIG. 5 is a schematic bottom view of the sensor unit 3.

The sensor unit 3 in FIG. 3 includes a sensor substrate 33 and a protective layer 34 in addition to the first electrode 31 and the second electrode 32. As illustrated in FIGS. 3 to 5, the sensor substrate 33 has a rectangular plate shape. The sensor substrate 33 has a first surface 33a and a second surface 33b in a thickness direction of the sensor substrate 33. The first electrode 31, the second electrode 32, and the protective layer 34 are disposed on the sensor substrate 33.

The first electrode 31 has an electrode portion 311, a terminal portion 312, and a connection portion 313. The electrode portion 311 is used for contact with the measurement target. As illustrated in FIG. 3, the electrode portion 311 is disposed on the first surface 33a of the sensor substrate 33. As illustrated in FIG. 4, the electrode portion 311 has a comb tooth structure. The electrode portion 311 includes a plurality of tooth portions 3111 arranged at a predetermined interval, and a coupling portion 3112 that couples one ends of the plurality of tooth portions 3111 to each other. As illustrated in FIG. 3, the electrode portion 311 includes a plurality of metal layers. The plurality of metal layers of the electrode portion 311 includes a Ni layer 311a, a Pd layer 311b that covers the Ni layer 311a, and an Au layer 311c that covers the Pd layer 311b. The plurality of metal layers of the electrode portion 311 can be formed by plating processing. The terminal portion 312 is used for the connection to the electrostatic capacity detection circuit 4. As illustrated in FIG. 3, the terminal portion 312 is disposed on the second surface 33b of the sensor substrate 33. As illustrated in FIG. 4, the terminal portion 312 has a rectangular plate shape. As illustrated in FIG. 3, the terminal portion 312 includes a plurality of metal layers (metal films). The plurality of metal layers of the terminal portion 312 includes a Ni layer 312a, a Pd layer 312b that covers the Ni layer 312a, and an Au layer 312c that covers the Pd layer 312b. The plurality of metal layers of the terminal portion 312 can be formed by plating processing. The connection portion 313 connects the electrode portion 311 and the terminal portion 312. As illustrated in FIG. 3, the connection portion 313 is a via that penetrates the sensor substrate 33. The connection portion 313 is made of, for example, Ag.

The second electrode 32 has an electrode portion 321, a terminal portion 322, and a connection portion 323. The electrode portion 321 is used for contact with the measurement target. As illustrated in FIG. 3, the electrode portion 321 is disposed on the first surface 33a of the sensor substrate 33. As illustrated in FIG. 4, the electrode portion 321 has a comb tooth structure. The electrode portion 321 includes a plurality of tooth portions 3211 arranged at a predetermined interval, and a coupling portion 3212 that couples one ends of the plurality of tooth portions 3211 to each other. As illustrated in FIG. 3, the electrode portion 321 includes a plurality of metal layers. The plurality of metal layers of the electrode portion 321 includes a Ni layer 321a, a Pd layer 321b that covers the Ni layer 321a, and an Au layer 321c that covers the Pd layer 321b. The plurality of metal layers of the electrode portion 321 can be formed by plating processing. The terminal portion 322 is used for the connection to the electrostatic capacity detection circuit 4. As illustrated in FIG. 3, the terminal portion 322 is disposed on the second surface 33b of the sensor substrate 33. As illustrated in FIG. 4, the terminal portion 322 has a rectangular plate shape. As illustrated in FIG. 3, the terminal portion 322 includes a plurality of metal layers (metal films). The plurality of metal layers of the terminal portion 322 includes a Ni layer 322a, a Pd layer 322b that covers the Ni layer 322a, and an Au layer 322c that covers the Pd layer 322b. The plurality of metal layers of the terminal portion 322 can be formed by plating processing. The connection portion 323 connects the electrode portion 321 and the terminal portion 322. As illustrated in FIG. 3, the connection portion 323 is a via that penetrates the sensor substrate 33. The connection portion 323 is made of, for example, Ag.

As illustrated in FIG. 3, the protective layer 34 is disposed on the first surface 33a of the sensor substrate 33. The protective layer 34 covers the electrode portion 311 of the first electrode 31 and the electrode portion 321 of the second electrode 32. The protective layer 34 protects the first electrode 31 and the second electrode 32. The protective layer 34 has, for example, insulating properties. The protective layer 34 is made of, for example, a material having insulating properties, such as polyimide.

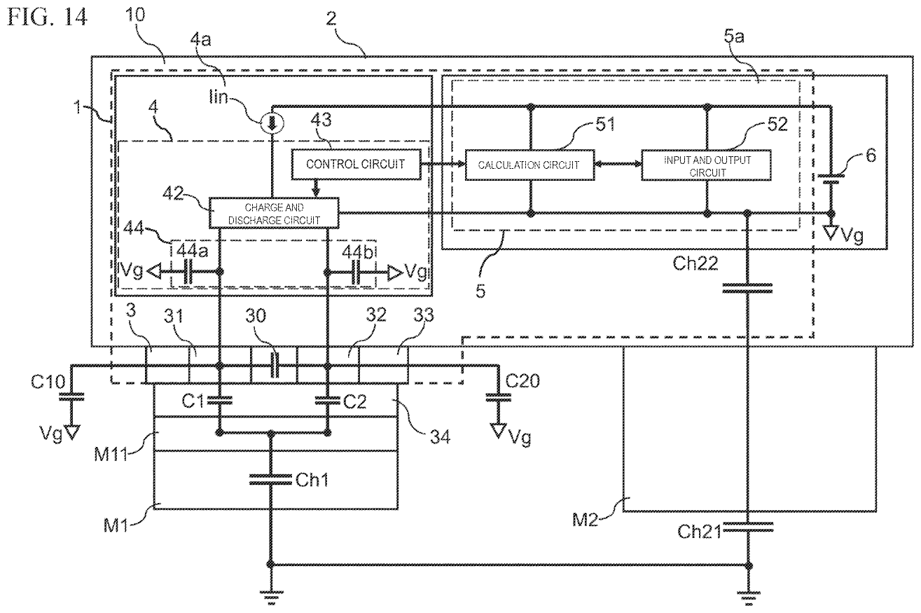

The electrostatic capacity detection circuit 4 in FIG. 2 detects an electrostatic capacity of the capacitor 30 based on a charge and discharge time of the capacitor 30 of the sensor unit 3. The electrostatic capacity detection circuit 4 includes a power supply terminal 41a that is connected to a power supply Iin, a reference potential terminal 41b that is connected to the reference potential Vg, a charge and discharge circuit 42, a control circuit 43, and an auxiliary capacity circuit 44. The charge and discharge circuit 42, the control circuit 43, and the auxiliary capacity circuit 44 are disposed on a circuit substrate 4a different from the sensor substrate 33 (see FIG. 14). The power supply Iin is disposed on the circuit substrate 4a.

The power supply Iin supplies power for charging the capacitor 30 to the electrostatic capacity detection circuit 4. The power supply Iin in FIG. 2 is a constant current source that outputs a constant output current to the electrostatic capacity detection circuit 4. The power supply Iin is operated by the power from the DC power supply 6. Since the power supply Iin may have a known configuration in the related art, the detailed description will be omitted.

The charge and discharge circuit 42 in FIG. 2 is connected to the first and second electrodes 31 and 32 constituting the capacitor 30, and is configured to charge and discharge the capacitor 30 of the sensor unit 3. The charge and discharge circuit 42 in FIG. 2 is connected between the power supply terminal 41a and the reference potential terminal 41b. The charge and discharge circuit 42 includes first to fourth switches S1 to S4.

The first switch S1 is connected between the first electrode 31 and the power supply terminal 41a. The second switch S2 is connected between the second electrode 32 and the power supply terminal 41a. The third switch S3 is connected between the first electrode 31 and the reference potential terminal 41b. The fourth switch S4 is connected between the second electrode 32 and the reference potential terminal 41b. In other words, in the charge and discharge circuit 42, the first switch S1 and the third switch S3 constitute a series circuit, the series circuit of the first switch S1 and the third switch S3 is connected between the power supply terminal 41a and the reference potential terminal 41b, and a connection point of the first switch S1 and the third switch S3 is connected to the first electrode 31. In other words, in the charge and discharge circuit 42, the second switch S2 and the fourth switch S4 constitute a series circuit, the series circuit of the second switch S2 and the fourth switch S4 is connected between the power supply terminal 41a and the reference potential terminal 41b, and a connection point of the second switch S2 and the fourth switch S4 is connected to the second electrode 32.

In the present embodiment, each of the first to fourth switches S1 to S4 is a field effect transistor. Each of the first to fourth switches S1 to S4 is, for example, a MOSFET. Here, the first and second switches S1 and S2 are enhancement type P-channel MOSFETs, and the third and fourth switches S3 and S4 are enhancement type N-channel MOSFETs.

The charge and discharge circuit 42 is configured to be complementarily switchable between a first state and a second state. The first state is a state where a constant current is supplied to the first electrode 31 of the sensor unit 3. In FIG. 2, the first state is a state where an output current from the power supply Iin is supplied to the first electrode 31. In the first state, the first and fourth switches S1 and S4 are turned on, and the second and third switches S2 and S3 are turned off. Thus, the first and second electrodes 31 and 32 are connected to the power supply terminal 41a and the reference potential terminal 41b, respectively. In the first state, the capacitor 30 is charged such that a potential of the first electrode 31 is higher than a potential of the second electrode 32. The second state is a state where a constant current is supplied to the second electrode 32 of the sensor unit 3. In FIG. 2, the second state is a state where the output current from the power supply Iin is supplied to the second electrode 32. In the second state, the first and fourth switches S1 and S4 are turned off, and the second and third switches S2 and S3 are turned on. Thus, the first and second electrodes 31 and 32 are connected to the reference potential terminal 41b and the power supply terminal 41a, respectively. In the second state, the capacitor 30 is charged such that the potential of the second electrode 32 is higher than the potential of the first electrode 31. Since power is supplied to the first and second electrodes 31 and 32 such that positive and negative of the first electrode 31 and the second electrode 32 are alternately switched, it can be said that the charge and discharge circuit 42 is an oscillation circuit.

When the charge and discharge circuit 42 is switched between the first state and the second state, in order to prevent overcurrent due to the fact that two switches (first and third switches S1 and S3 or the second and fourth switches S2 and S4) connected in series are short-circuited, a dead time is provided by setting the charge and discharge circuit to a third state where all of the first switch S1 to the fourth switch S4 are turned off. When the charge and discharge circuit is switched between the first state and the second state, the charge and discharge circuit 42 controls the first to fourth switches S1 to S4 such that first state->third state->second state or second state->third state->first state.

The auxiliary capacity circuit 44 in FIG. 2 includes first and second auxiliary capacitors 44a and 44b. The first and second auxiliary capacitors 44a and 44b are provided to reduce influence of stray capacitance on the detection of the electrostatic capacity of the capacitor 30. A first end of the first auxiliary capacitor 44a is connected to the first electrode 31 and a second end of the first auxiliary capacitor 44a is connected to the reference potential terminal 41b such that the first auxiliary capacitor 44a is in parallel with the capacitor 30. In the present embodiment, the first auxiliary capacitor 44a is connected in parallel with the third switch S3. As a result, the first auxiliary capacitor 44a is connected between the first electrode 31 and the reference potential terminal 41b. A first end of the second auxiliary capacitor 44b is connected to the second electrode 32 and a second end of the second auxiliary capacitor 44b is connected to the reference potential terminal 41b such that the second auxiliary capacitor 44b is in parallel with the capacitor 30. In the present embodiment, the second auxiliary capacitor 44b is connected in parallel with the fourth switch S4. As a result, the second auxiliary capacitor 44b is connected between the second electrode 32 and the reference potential terminal 41b.

Electrostatic capacities of the first and second auxiliary capacitors 44a and 44b may be set, for example, based on a measurable range of the electrostatic capacity of the capacitor 30. The measurable range of the electrostatic capacity of the capacitor 30 is appropriately set based on the measurement target. As an example, the electrostatic capacities of the first and second auxiliary capacitors 44a and 44b may be set to five times any value within the measurable range of the electrostatic capacity of the capacitor 30. Here, any value may be an upper limit value. In the case of the measurement of the moisture content in the oral cavity, as an example, the upper limit value may be 9.4 pF, and the electrostatic capacities of the first and second auxiliary capacitors 44a and 44b may be 47 pF. In the present embodiment, the electrostatic capacities of the first and second auxiliary capacitors 44a and 44b are equal to each other.

The auxiliary capacity circuit 44 is disposed between the sensor substrate 33 of the sensor unit 3 and a circuit substrate 5a and at a position closer to the circuit substrate 5a than the sensor substrate 33. In the present embodiment, the auxiliary capacity circuit 44 is disposed on the circuit substrate 5a. Since the sensor unit 3 is a contact portion that is brought into contact with the measurement target in the electrostatic capacity sensor 1, the influence of the stray capacitance can be suppressed as the sensor is farther from the contact portion.

Accordingly, the influence of the stray capacitance from the first electrode 31 and the second electrode 32 of the sensor unit 3 can be reduced.

The control circuit 43 in FIG. 2 is configured to control the charge and discharge circuit 42 such that the capacitor 30 of the sensor unit 3 repeats charge and discharge. In the present embodiment, the control circuit 43 controls the charge and discharge circuit 42 such that the charge and discharge circuit 42 alternately switches between the first state and the second state.

Hereinafter, the control circuit 43 will be described in further detail. The control circuit 43 in FIG. 2 has a determination circuit 431 and a drive circuit 432.

The determination circuit 431 is configured to determine a timing of switching between charge and discharge of the capacitor 30 of the sensor unit 3. The timing of switching between the charge and discharge of the capacitor 30 is a timing of switching between the first state and the second state of the charge and discharge circuit 42. The determination circuit 431 determines the timing of switching between the charge and discharge of the capacitor 30 of the sensor unit 3 based on the potential of the first electrode 31 and the potential of the second electrode 32. The determination circuit 431 executes determination as to whether or not the potential of the first electrode 31 reaches a first threshold in a case where the charge and discharge circuit 42 is in the first state. The determination circuit 431 executes determination as to whether or not the potential of the second electrode 32 reaches a second threshold when the charge and discharge circuit 42 is in the second state. In the present embodiment, the first threshold and the second threshold are equal to each other. The determination result of the determination circuit 431 is outputted to the drive circuit 432. The determination circuit 431 may include, for example, a first comparator that compares the potential of the first electrode 31 and the first threshold, a second comparator that compares the potential of the second electrode 32 and the second threshold, and an OR circuit to which output signals from the first and second comparator are inputted.

The drive circuit 432 is configured to drive the first to fourth switches S1 to S4 of the charge and discharge circuit 42 in accordance with the determination result of the determination circuit 431. In the present embodiment, the drive circuit 432 outputs a first drive signal D1 common to the first and third switches S1 and S3, and outputs a second drive signal D2 common to the second and fourth switches S2 and S4. As described above, the first and second switches S1 and S2 are the enhancement type P-channel MOSFETs, and the third and fourth switches S3 and S4 are the enhancement type N-channel MOSFETs. In driving the first to fourth switches S1 to S4, a voltage value of the first drive signal D1 and a voltage value of the second drive signal D2 are set to a high level or a low level. The high level and the low level are determined from characteristics of the enhancement type P-channel MOSFETs of the first and second switches S1 and S2 and the enhancement type N-channel MOSFETs of the third and fourth switches S3 and S4. The high level and the low level are set such that the first switch S1 is turned on and the third switch S3 is turned off when the first drive signal D1 is at the high level and the first switch S1 is turned off and the third switch S3 is turned on when the first drive signal D1 is at the low level. The high level and the low level are set such that the second switch S2 is turned on and the fourth switch S4 is turned off when the second drive signal D2 is at the high level, and the second switch S2 is turned off and the fourth switch S4 is turned on when the second drive signal D2 is at the low level. The first drive signal D1 and the second drive signal D2 are prevented from being at the high level or low level at the same time.

In a case where the charge and discharge circuit 42 is set to the first state, the drive circuit 432 sets the voltage value of the first drive signal D1 to the high level and the voltage value of the second drive signal D2 to the low level. As a result, the first and fourth switches S1 and S4 are turned on, and the second and third switches S2 and S3 are turned off. In a case where the charge and discharge circuit 42 is set to the second state, the drive circuit 432 sets the voltage value of the first drive signal D1 to the low level and the voltage value of the second drive signal D2 to the high level. As a result, the first and fourth switches S1 and S4 are turned off, and the second and third switches S2 and S3 are turned on.

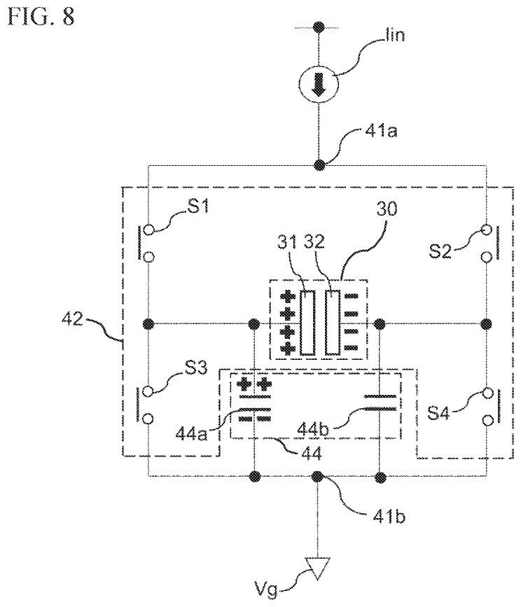

In a state where the voltage value of the first drive signal D1 is set to the low level and the voltage value of the second drive signal D2 is set to the high level, when the determination circuit 431 determines that the potential of the first electrode 31 reaches the first threshold, the drive circuit 432 sets the voltage value of the first drive signal D1 is set to the low level and the voltage value of the second drive signal D2 to the high level. As a result, the charge and discharge circuit 42 is switched from the first state to the second state. In a state where the voltage value of the first drive signal D1 is set to the low level and the voltage value of the second drive signal D2 is set to the high level, when the determination circuit 431 determines that the potential of the second electrode 32 reaches the second threshold, the drive circuit 432 sets the voltage value of the first drive signal D1 to the high level and the voltage value of the second drive signal D2 to the low level. As a result, the charge and discharge circuit 42 is switched from the second state to the first state. Note that, when the drive circuit 432 switches the voltage values of the first and second drive signals D1 and D2 between the high level and the low level, a dead time is provided by setting the charge and discharge circuit to the third state as described above. For example, in a procedure in which the voltage value of the first drive signal D1 is switched between the high level and the low level and the voltage value of the second drive signal D2 is switched between the high level and the low level, the drive circuit 432 sets the voltage value of the first drive signal D1 and the voltage value of the second drive signal D2 to an intermediate voltage at which all of the first to fourth switches S1 to S4 are turned off as illustrated in FIG. 8. As a result, a possibility of being short-circuited between the power supply Iin and the reference potential Vg in the charge and discharge circuit 42 is reduced.

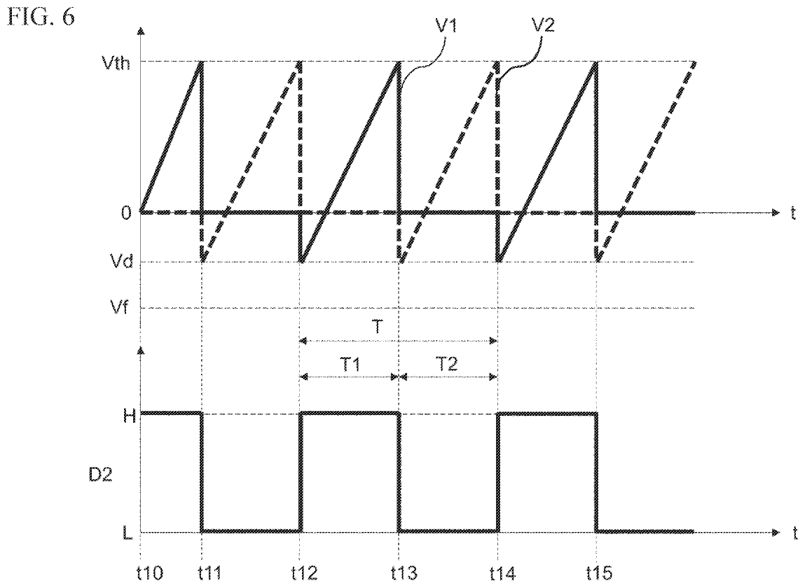

Next, an example of an operation of the electrostatic capacity detection circuit 4 will be described with reference to FIGS. 6 to 13.

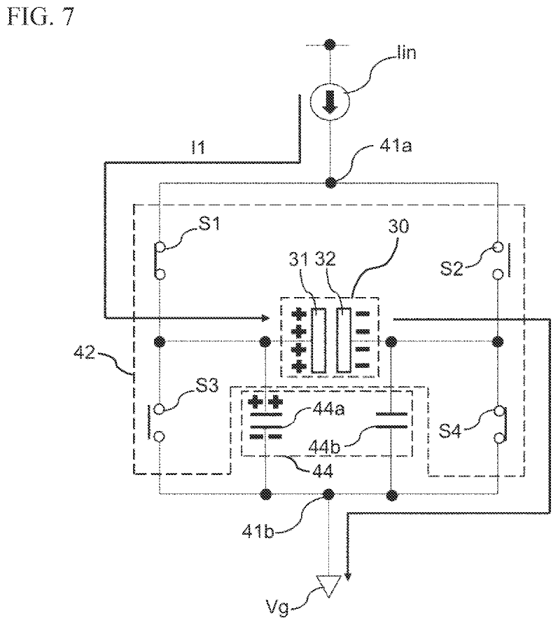

FIG. 6 is a timing chart of an example of the operation of the electrostatic capacity detection circuit 4. In FIG. 6, V1 indicates the potential of the first electrode 31, and V2 indicates the potential of the second electrode 32. In FIG. 6, H corresponds to a state where the voltage value of the second drive signal D2 is at the high level, and L indicates a state where the voltage value of the second drive signal D2 is at the low level. FIGS. 7 to 13 are explanatory diagrams of an example of the operation of the electrostatic capacity detection circuit 4. In FIGS. 7 to 13, the control circuit 43 is omitted only for simplification of the drawings.

At time t10 in FIG. 6, charges are not accumulated in the capacitor 30. The drive circuit 432 sets the charge and discharge circuit 42 to the first state by setting the voltage value of the first drive signal D1 to the high level and the voltage value of the second drive signal D2 to the low level.

FIG. 7 is an explanatory diagram of the operation of the electrostatic capacity detection circuit 4 when the charge and discharge circuit 42 is in the first state. As illustrated in FIG. 7, in the first state, the first and fourth switches S1 and S4 are turned on, and the second and third switches S2 and S3 are turned off. A constant output current I1 is supplied from the power supply Iin to the first electrode 31. As a result, the capacitor 30 is charged such that the potential V1 of the first electrode 31 is higher than the potential V2 of the second electrode 32. Since the charge and discharge circuit 42 has the first auxiliary capacitor 44a connected in parallel with the first electrode 31, the first auxiliary capacitor 44a is connected in parallel with the capacitor 30 in the first state, and charges are also accumulated in the first auxiliary capacitor 44a.

The determination circuit 431 executes determination as to whether or not the potential V1 of the first electrode 31 reaches the first threshold in a case where the charge and discharge circuit 42 is in the first state. In FIG. 6, the first threshold is Vth. At time t11, the determination circuit 431 determines that the potential V1 of the first electrode 31 reaches the first threshold (Vth). As a result, the drive circuit 432 sets the charge and discharge circuit 42 to the second state. In the present embodiment, the drive circuit 432 provides a dead time by setting the charge and discharge circuit 42 to the third state when the charge and discharge circuit 42 is switched from the first state to the second state. More specifically, in a procedure in which the voltage value of the first drive signal D1 is set to the low level from the high level and the voltage value of the second drive signal D2 is set to the high level from the low level, the drive circuit 432 sets the voltage value of the first drive signal D1 and the voltage value of the second drive signal D2 to an intermediate voltage at which all of the first to fourth switches S1 to S4 are turned off as illustrated in FIG. 8. FIG. 8 is an explanatory diagram of the operation of the electrostatic capacity detection circuit 4 when the charge and discharge circuit 42 is in the third state. Thereafter, the drive circuit 432 sets the charge and discharge circuit 42 to the second state by setting the voltage value of the first drive signal D1 to the low level and the voltage value of the second drive signal D2 to the high level.

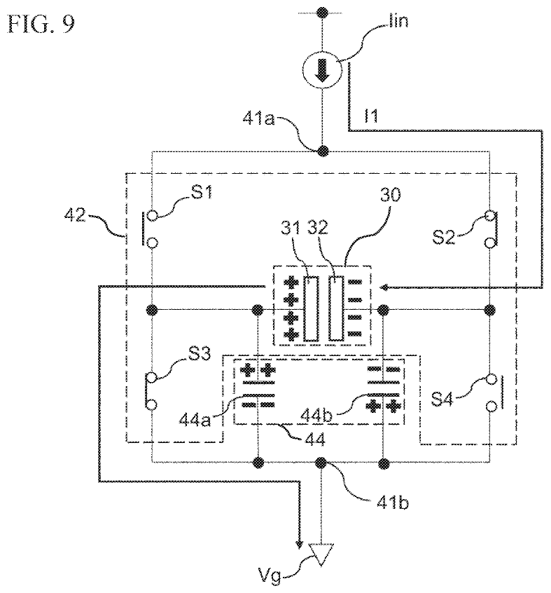

FIG. 9 is an explanatory diagram of the operation of the electrostatic capacity detection circuit 4 immediately after the charge and discharge circuit 42 is switched to the second state. As illustrated in FIG. 9, in the second state, the first and fourth switches S1 and S4 are turned off, and the second and third switches S2 and S3 are turned on. In the capacitor 30, the first electrode 31 is connected to the reference potential terminal 41b, and the second electrode 32 is connected to the power supply terminal 41a. Immediately after the charge and discharge circuit 42 is switched to the second state, the potential V2 of the second electrode 32 is negative. Since the charge and discharge circuit 42 has the second auxiliary capacitor 44b connected in parallel with the second electrode 32, the second auxiliary capacitor 44b is connected in parallel with the capacitor 30 in the second state. As a result, the charges of the capacitor 30 move to the second auxiliary capacitor 44b. In FIG. 6, the potential V2 of the second electrode 32 decreases to Vd. Vd is a negative value. Since Vd is determined by the charges stored in the capacitor 30 in the first state and a combined electrostatic capacity of the capacitor 30 and the second auxiliary capacitor 44b, the following Equation (1) is established.

$$\begin{matrix}[ Math . 1 ] & \\ \vert Vd \vert = \frac{Ce}{Ce + Cg} \cdot \vert Vth \vert & ( 1 )\end{matrix}$$

In Equation (1), Ce is the electrostatic capacity of the capacitor 30, and Cg is the electrostatic capacities of the first auxiliary capacitor 44a and the second auxiliary capacitor 44b.

In the present embodiment, the electrostatic capacity (Cg) of the second auxiliary capacitor 44b and the first threshold (Vth) are set such that |Vd|≤|Vf| is established. Vf is a negative value, and the magnitude of Vf is equal to a threshold voltage of a body diode of a field effect transistor used as the third switch S3. In the case of |Vd|>|Vf|, since a forward voltage of the body diode exceeds |Vf|, the potential V2 of the second electrode 32 decreases to Vf. The magnitude of Vf corresponds to the magnitude of a threshold voltage of a body diode of the field effect transistor of the third switch S3, and is a lower limit value of the potential V2 of the second electrode 32 when the charge and discharge circuit 42 in a case where there is not the second auxiliary capacitor 44b is switched from the first state to the second state.

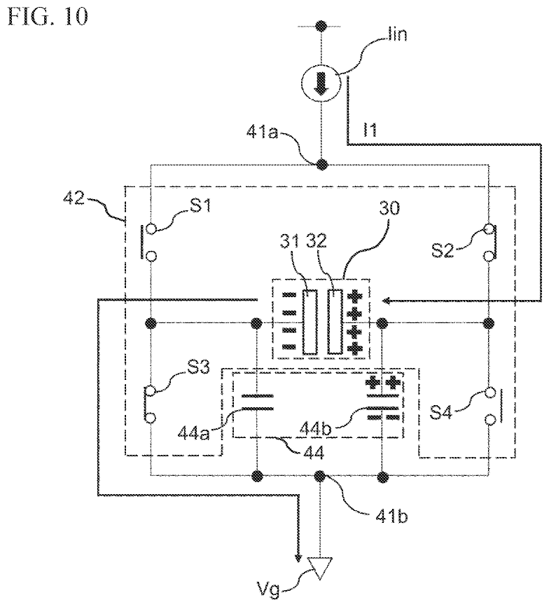

In the second state, the constant output current I1 is supplied to the second electrode 32 from the power supply Iin. As a result, the capacitor 30 is charged such that the potential V2 of the second electrode 32 is higher than the potential V1 of the first electrode 31. In addition, charges are also accumulated in the second auxiliary capacitor 44b. FIG. 10 is an explanatory diagram of the operation of the electrostatic capacity detection circuit 4 when a time elapses since the charge and discharge circuit 42 is switched to the second state. In FIG. 10, the potential V2 of the second electrode 32 is positive.

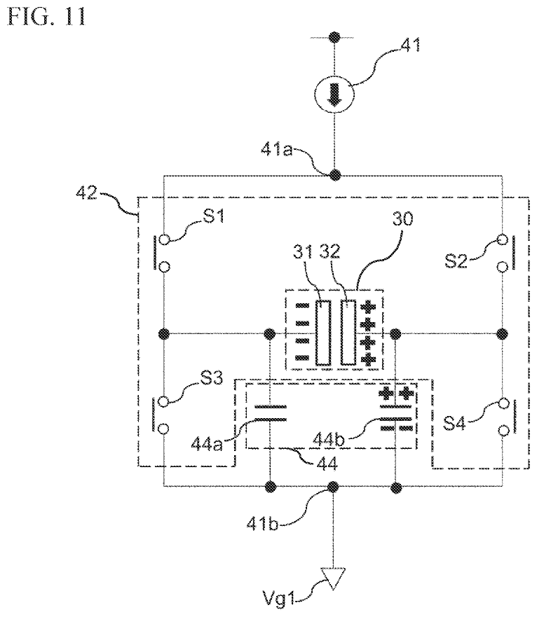

The determination circuit 431 executes determination as to whether or not the potential V2 of the second electrode 32 reaches the second threshold in a case where the charge and discharge circuit 42 is in the second state. In FIG. 6, the second threshold is equal to the first threshold and is Vth. At time t12, the determination circuit 431 determines that the potential V2 of the second electrode 32 reaches the second threshold (Vth). As a result, the drive circuit 432 sets the charge and discharge circuit 42 to the first state. In the present embodiment, the drive circuit 432 provides a dead time by setting the charge and discharge circuit 42 to the third state when the charge and discharge circuit 42 is switched from the second state to the first state. FIG. 11 is an explanatory diagram of the operation of the electrostatic capacity detection circuit 4 when the charge and discharge circuit 42 is in the third state. Thereafter, the drive circuit 432 sets the charge and discharge circuit 42 to the first state by setting the voltage value of the first drive signal D1 to the high level and the voltage value of the second drive signal D2 to the low level.

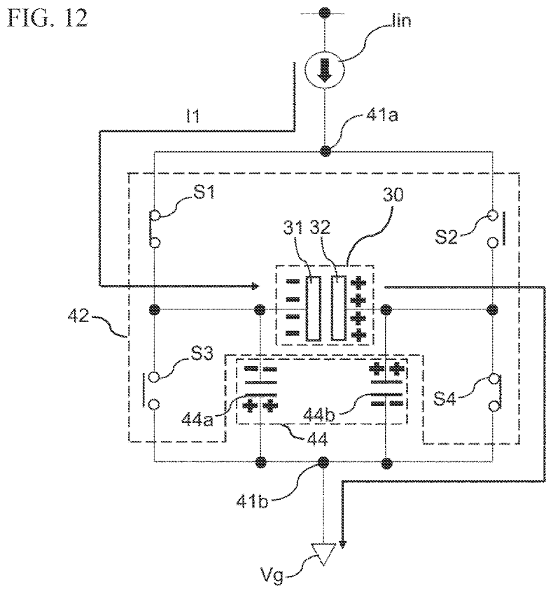

FIG. 12 is an explanatory diagram of the operation of the electrostatic capacity detection circuit 4 immediately after the charge and discharge circuit 42 is switched to the first state. As illustrated in FIG. 12, in the first state, the first and fourth switches S1 and S4 are turned on, and the second and third switches S2 and S3 are turned off. In the capacitor 30, the first electrode 31 is connected to the power supply terminal 41a, and the second electrode 32 is connected to the reference potential terminal 41b. Immediately after the charge and discharge circuit 42 is switched to the first state, the potential V1 of the first electrode 31 is negative. Since the charge and discharge circuit 42 has the first auxiliary capacitor 44a connected in parallel with the first electrode 31, the first auxiliary capacitor 44a is connected in parallel with the capacitor 30 in the first state. As a result, the charges of the capacitor 30 move to the first auxiliary capacitor 44a. In FIG. 6, the potential V1 of the first electrode 31 decreases to Vd, as with the second electrode 32.

In the present embodiment, the electrostatic capacity (Cg) of the first auxiliary capacitor 44a and the second threshold (Vth) are set such that |Vd|≤|Vf| is established. Vf is a negative value, and the magnitude of Vf is equal to a threshold voltage of a body diode of a field effect transistor used as the fourth switch S4. In the case of |Vd|>|Vf|, since the forward voltage of the body diode exceeds |Vf|, the potential V1 of the first electrode 31 decreases to Vf. The magnitude of Vf corresponds to the magnitude of a threshold voltage of a body diode of the field effect transistor of the fourth switch S4, and is a lower limit value of the potential V1 of the first electrode 31 when the charge and discharge circuit 42 in a case where there is not the first auxiliary capacitor 44a is switched from the second state to the first state.

In the first state, the constant output current I1 is supplied from the power supply Iin to the first electrode 31. As a result, the capacitor 30 is charged such that the potential V1 of the first electrode 31 is higher than the potential V2 of the second electrode 32. In addition, charges are also accumulated in the first auxiliary capacitor 44a. After a time elapses since the charge and discharge circuit 42 is switched to the first state, the potential V1 of the first electrode 31 is positive as illustrated in FIG. 7.

At time t13 in FIG. 6, the determination circuit 431 determines that the potential V1 of the first electrode 31 reaches the first threshold (Vth). As a result, the drive circuit 432 sets the charge and discharge circuit 42 to the second state. As a result, the capacitor 30 is charged such that the potential V2 of the second electrode 32 is higher than the potential V1 of the first electrode 31.

At time t14 in FIG. 6, the determination circuit 431 determines that the potential V2 of the second electrode 32 reaches the second threshold (Vth). As a result, the drive circuit 432 sets the charge and discharge circuit 42 to the first state. As a result, the capacitor 30 is charged such that the potential V1 of the first electrode 31 is higher than the potential V2 of the second electrode 32.

At time t15 in FIG. 6, as with the case of time t13, the determination circuit 431 determines that the potential V1 of the first electrode 31 reaches the first threshold (Vth), and the drive circuit 432 sets the charge and discharge circuit 42 to the second state. As a result, the capacitor 30 is charged such that the potential V2 of the second electrode 32 is higher than the potential V1 of the first electrode 31.

As described above, in a case where the charge and discharge circuit 42 is in the first state, when the potential V1 of the first electrode 31 reaches the first threshold (Vth), the control circuit 43 switches the charge and discharge circuit 42 from the first state to the second state. In a case where the charge and discharge circuit 42 is in the second state, when the potential V2 of the second electrode 32 reaches the second threshold (Vth), the control circuit 43 switches the charge and discharge circuit 42 from the second state to the first state. Accordingly, in the electrostatic capacity detection circuit 4, a state where the capacitor 30 is charged such that the potential of the first electrode 31 is higher than the potential of the second electrode 32 and a state where the capacitor 30 is charged such that the potential of the second electrode 32 is higher than the potential of the first electrode 31 are repeated.

In FIG. 6, T indicates a period of charging and discharging of the capacitor 30. The period T is the sum of a first period T1 and a second period T2. The first period T1 is a length of a period of time during which the charge and discharge circuit 42 is in the first state. The length of the period of time during which the charge and discharge circuit 42 is in the first state is a time taken for the potential of the first electrode 31 to be from Vd to Vth by supplying the constant output current I1 from the power supply Iin to a combined capacitor of the capacitor 30 and the first auxiliary capacitor 44a. The second period T2 is a length of a period of time during which the charge and discharge circuit 42 is in the second state. The length of the period of time during which the charge and discharge circuit 42 is in the second state is a time taken for the potential of the second electrode 32 to be from Vd to Vth by supplying the constant output current I1 from the power supply Iin to a combined capacitor of the capacitor 30 and the second auxiliary capacitor 44b. Accordingly, the period T is given by the following Equation (2). In the following Equation (2), i is a value (current value) of the output current I1.

Note that, in a case where the electrostatic capacity of the first auxiliary capacitor 44a and the electrostatic capacity of the second auxiliary capacitor 44b are equal, as illustrated in FIG. 6, the period T1 and the period T2 are equal, and the potential Vd in the period T1 and the potential Vd in the period T2 are equal. Accordingly, periods of time of the periods T1 and T2 or the potential Vd thereof are measured, and thus, it is possible to calculate the electrostatic capacity of the first auxiliary capacitor 44a and the electrostatic capacity of the second auxiliary capacitor 44b.

$$\begin{matrix}[ Math . 2 ] & \\ T = T 1 + T 2 = 2 \cdot \frac{( Vth + Vd ) \cdot ( Ce + Cg )}{i} & ( 2 )\end{matrix}$$

When Vd of the above Equation (1) is substituted into Equation (2), the following Equation (3) is obtained.

$$\begin{matrix}[ Math . 3 ] & \\ T = \frac{2 \cdot Vth}{i} \cdot ( 2 Ce + Cg ) & ( 3 )\end{matrix}$$

As is clear from the above Equation (3), it is possible to calculate the electrostatic capacity Ce of the capacitor 30 from the period T.

Note that, in the present embodiment, the electrostatic capacity Ce is calculated from the equation including the period T, but the present disclosure is not limited thereto, and the electrostatic capacity may be measured by an existing method such as impedance measurement.

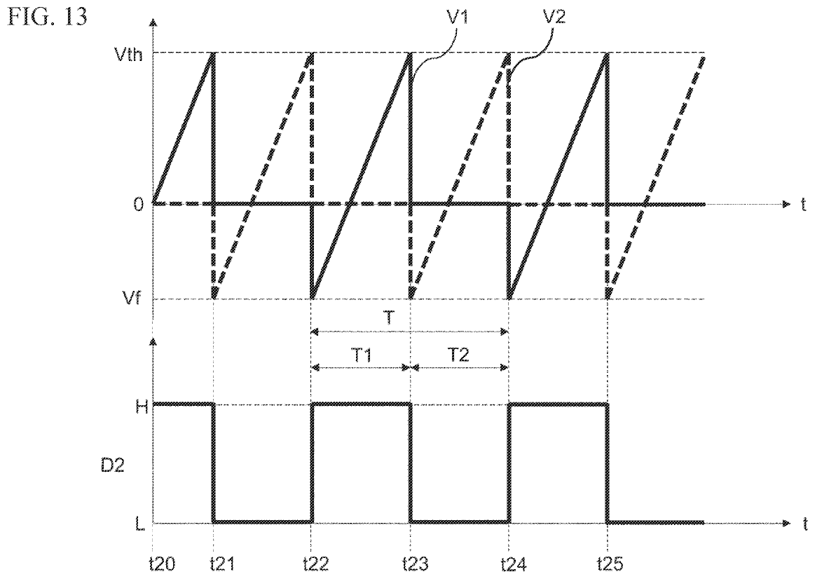

FIG. 13 is a timing chart of an example of an operation of an electrostatic capacity detection circuit of a comparative example. The electrostatic capacity detection circuit of the comparative example is different from the electrostatic capacity detection circuit 4 in that the first and second auxiliary capacitors 44a and 44b are not provided.

In FIG. 13, the charge and discharge circuit 42 is set to the first state at time t20, and the capacitor 30 is charged such that the potential V1 of the first electrode 31 is higher than the potential V2 of the second electrode 32. At time t21, the potential V1 reaches the first threshold Vth, and the charge and discharge circuit 42 is switched to the second state. At time t22, the potential V2 reaches the second threshold Vth, and the charge and discharge circuit 42 is switched to the first state. At time t23, the potential V1 reaches the first threshold Vth, and the charge and discharge circuit 42 is switched to the second state. At time t24, the potential V2 reaches the second threshold Vth, and the charge and discharge circuit 42 is switched to the first state. At time t25, the potential V1 reaches the first threshold Vth, and the charge and discharge circuit 42 is switched to the second state.

As illustrated in FIG. 13, immediately after the charge and discharge circuit 42 is switched to the first state (see times t22 and t24), the potential V1 of the first electrode 31 is Vf. Immediately after the charge and discharge circuit 42 is switched to the second state (see times t21 and t23), the potential V2 of the second electrode 32 is Vf. This is because |Vth| is larger than |Vf|.

In the comparative example, the first period T1 is a time taken for the potential of the first electrode 31 to be Vth from Vf by supplying the constant output current I1 from the power supply Iin to the capacitor 30. The second period T2 is a time taken for the potential of the second electrode 32 to be Vth from Vf by supplying the constant output current I1 from the power supply Iin to the capacitor 30. Accordingly, in the comparative example, the period T is given by the following Equation (4).

$$\begin{matrix}[ Math . 4 ] & \\ T = T 1 + T 2 = \frac{2 \cdot ( Vth + Vf )}{i} \cdot Ce & ( 4 )\end{matrix}$$

When Equation (3) and Equation (4) are compared, in the electrostatic capacity detection circuit 4 of the present embodiment, it can be seen that the influence of a change in the electrostatic capacity Ce in the period T is two times as much as that of the electrostatic capacity detection circuit of the comparative example. The electrostatic capacity detection circuit 4 can reduce the influence of the stray capacitance on the detection of the electrostatic capacity.

The processing circuit 5 in FIG. 2 includes a calculation circuit 51 and an input and output circuit 52. The calculation circuit 51 and the input and output circuit 52 are disposed on the circuit substrate 5a different from the sensor substrate 33 and the circuit substrate 4a (see FIG. 14). The reference potential Vg is provided on the circuit substrate 5a. The DC power supply 6 is disposed on the circuit substrate 5a.

The input and output circuit 52 has a function as an input device for operating the electrostatic capacity sensor 1 and an output device for outputting information from the electrostatic capacity sensor 1. The input and output circuit 52 includes, for example, one or more human-machine interfaces. Examples of the human-machine interface include an input device such as a mechanical switch or a touch pad, an output device such as a display or a speaker, and an input and output device such as a touch panel.

The calculation circuit 51 controls the operation of the electrostatic capacity sensor 1. The calculation circuit 51 is connected to the input and output circuit 52. The calculation circuit 51 can be realized by, for example, a computer system including one or more processors (microprocessors) and one or more memories. The function as the calculation circuit 51 is realized by one or more processors (such as one or more memories) executing a program.

The calculation circuit 51 is connected to the input and output circuit 52. In a case where an operation to start measuring the moisture content is performed by the input device of the input and output circuit 52, the calculation circuit 51 causes the electrostatic capacity detection circuit 4 to start an operation for detecting the electrostatic capacity. The calculation circuit 51 is configured to calculate the electrostatic capacity of the capacitor 30 based on a charging and discharging time of the capacitor 30 by the electrostatic capacity detection circuit 4. In the present embodiment, the charging and discharging time during which the capacitor 30 is charged and discharged by the electrostatic capacity detection circuit 4 is the period T. The calculation circuit 51 in FIG. 2 acquires the second drive signal D2 from the drive circuit 432 of the electrostatic capacity detection circuit 4, and determines the period T based on the second drive signal D2. As illustrated in FIG. 6, the period T corresponds to a period of the second drive signal D2. The calculation circuit 51 can obtain the electrostatic capacity Ce of the capacitor 30 from the period T based on the above Equation (3). The calculation circuit 51 is configured to obtain the moisture content of the measurement target based on the electrostatic capacity Ce of the capacitor 30. The calculation circuit 51 displays the moisture content of the measurement target by the output device of the input and output circuit 52.

### 1.1.2 Use Method

Next, an example of a method of using the measuring instrument 10 in FIG. 1 will be described. For example, the measuring instrument 10 is used by a measurer to measure the moisture content in the oral cavity of the measurement target. The measurer is, for example, a medical professional such as a doctor or a nurse. The measurement target is, for example, a patient. As an example, the measurer holds the grip portion 22 of the handheld housing 2 of the measuring instrument 10 with hand, puts the head portion 21 of the handheld housing 2 of the measuring instrument 10 into the oral cavity of the measurement target, and brings the head portion into contact with a measurement portion, such as lingual mucous membrane, buccal mucosa membrane, palatine mucous membrane, or labial mucous membrane. Since the measuring instrument 10 itself is not grounded, in a case where the measuring instrument 10 is used as described above, the head portion 21 of the measuring instrument 10 is grounded via the measurement target, and the grip portion 22 of the measuring instrument 10 is grounded via the measurer. Thus, when the measuring instrument 10 is used, various stray capacitances may be generated.

FIG. 14 is an explanatory diagram of stray capacitances generated when the measuring instrument 10 of FIG. 1 is used. In FIG. 14, M1 schematically indicates a body of the measurement target. M11 schematically indicates a body surface moisture layer of a measurer. M2 schematically indicates a body of the measurer.

As illustrated in FIG. 14, the head portion 21 of the handheld housing 2 of the measuring instrument 10 comes into contact with a specific part of an oral cavity of the measurement target M1. In FIG. 14, the first and second electrodes 31 and 32 of the sensor unit 3 touch the body surface moisture layer M11 of the measurement target M1 with the protective layer 34 interposed therebetween. As a result, a stray capacitance C1 can be generated between the first electrode 31 and the measurement target M1. A stray capacitance C2 can be generated between the second electrode 32 and the measurement target M1. The electrostatic capacity of the capacitor 30 including the first and second electrodes 31 and 32 changes under the influence of the stray capacitances C1 and C2.

When the measuring instrument 10 is used, stray capacitances unrelated to the electrostatic capacity to be measured are generated. In FIG. 14, a stray capacitance ch1 can be generated between the measurement target M1 and the ground. A stray capacitance Cp1 can be generated between a terminal connected to the first electrode 31 and the reference potential Vg in the electrostatic capacity detection circuit 4. A stray capacitance Cp2 can be generated between a terminal connected to the second electrode 32 and the reference potential Vg in the electrostatic capacity detection circuit 4. The grip portion 22 of the handheld housing 2 of the measuring instrument 10 is held by the measurer M2. In FIG. 14, a stray capacitance Ch21 can be generated between the measurer M2 and the ground. A stray capacitance Ch22 can be generated between the measurer M2 and the reference potential Vg of the processing circuit 5.

In order to measure the moisture content in this manner, in the measuring instrument 10 that comes into contact with the human body and measures the electrostatic capacity, even in a portion other the sensor unit 3 as a measurement portion, stray capacitances Ch1, Ch21, and Ch22 generated between the human body and the measuring instrument 10 and between the human body and the reference potential Vg are connected with a ground potential interposed therebetween, and there is a possibility that accurate capacitance cannot be observed. In a case where an electrostatic capacity to be measured is large, since the influence of such a stray capacitance is relatively small, the influence on measurement accuracy is small. In contrast, in a minute case such as the electrostatic capacity between the first and second electrodes 31 and 32, the influence of the stray capacitance can be a cause of a large error. In the related art, as a countermeasure against such a stray capacitance, there is a countermeasure in such a manner that it is necessary to minimize an area of a substrate having a circuit that converts an electrostatic capacity into a frequency and to isolate a reference potential from another functional circuit and it is possible to ignore a capacitance other than the measurement target from the circuit by a method such as a guard ring. However, an expensive and complicated circuit configuration is required to isolate the reference potential, it is difficult to insert the head portion into the oral cavity due to an increase in the substrate area, and the product price also increases. As for the guard ring, it is also necessary to electrically couple the human body to a product, and a degree of difficulty increases in terms of device configuration, safety, and the like.

In contrast, in the present embodiment, when the stray capacitances unrelated to the electrostatic capacity to be measured are collectively referred to as Cs, the above Equation (3) can be modified as the following Equation (5).

$$\begin{matrix}[ Math . 5 ] & \\ T = \frac{2 \cdot Vth}{i} \cdot ( 2 Ce + Cg + Cs ) & ( 5 )\end{matrix}$$

In the measuring instrument 10, the electrostatic capacity detection circuit 4 includes the first and second auxiliary capacitors 44a and 44b, and sensitivity can be relatively increased with respect to a capacitance to be detected by the electrostatic capacities Cg of the first and second auxiliary capacitors 44a and 44b. In other words, it is less likely to be influenced by the stray capacitance (for example, Ch1, Ch21, and Ch22 in FIG. 14) as a disturbance. The influence of the stray capacitance Cs can be reduced. In other words, in the present embodiment, the influence of the stray capacitance Cs can be reduced by simply disposing the first and second auxiliary capacitors 44a and 44b.

### 1.1.3 Modification Example

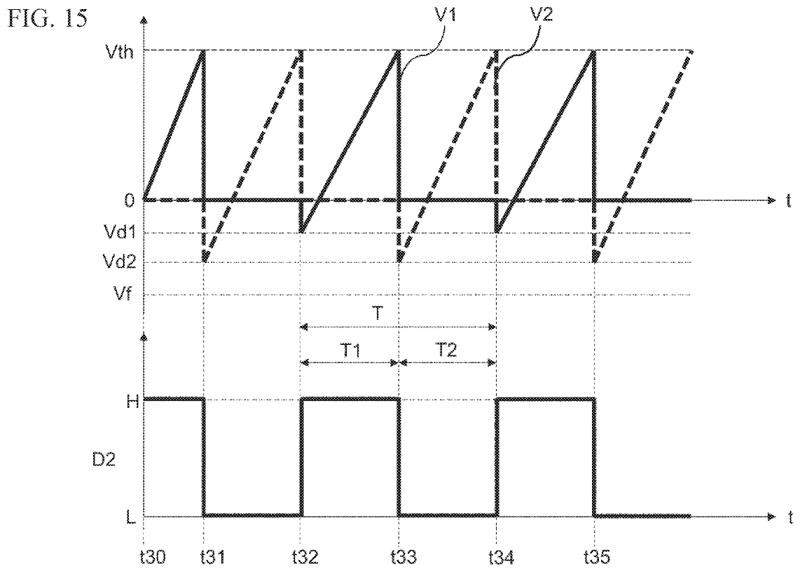

FIG. 15 is a timing chart of an example of an operation of the electrostatic capacity detection circuit according to a modification example of the first embodiment. The present modification example is different from the above configuration in that the electrostatic capacity of the first auxiliary capacitor 44a and the electrostatic capacity of the second auxiliary capacitor 44b are not the same but are different.

At time t30 in FIG. 15, charges are not accumulated in the capacitor 30. The drive circuit 432 sets the charge and discharge circuit 42 to the first state by setting the voltage value of the first drive signal D1 to the high level and the voltage value of the second drive signal D2 to the low level.

In the first state, the constant output current I1 is supplied from the power supply Iin to the first electrode 31. As a result, the capacitor 30 is charged such that the potential V1 of the first electrode 31 is higher than the potential V2 of the second electrode 32. Since the charge and discharge circuit 42 has the first auxiliary capacitor 44a connected in parallel with the first electrode 31, the first auxiliary capacitor 44a is connected in parallel with the capacitor 30 in the first state, and charges are also accumulated in the first auxiliary capacitor 44a.

At time t31, the determination circuit 431 determines that the potential V1 of the first electrode 31 reaches the first threshold (Vth). As a result, the drive circuit 432 sets the charge and discharge circuit 42 to the second state. Immediately after the charge and discharge circuit 42 is switched to the second state, the potential V2 of the second electrode 32 is negative. Since the charge and discharge circuit 42 has the second auxiliary capacitor 44b connected in parallel with the second electrode 32, the second auxiliary capacitor 44b is connected in parallel with the capacitor 30 in the second state. As a result, the charges of the capacitor 30 move to the second auxiliary capacitor 44b. In FIG. 15, the potential V2 of the second electrode 32 decreases to Vd2. Vd2 is a negative value. Since Vd2 is determined by the charges stored in the capacitor 30 in the first state and the combined electrostatic capacity of the capacitor 30 and the second auxiliary capacitor 44b, the following Equation (6) is established.

$$\begin{matrix}[ Math . 6 ] & \\ \vert Vd 2 \vert = \frac{Ce}{Ce + Cg 2} \cdot \vert Vth \vert & ( 6 )\end{matrix}$$

In Equation (6), Ce is the electrostatic capacity of the capacitor 30, and Cg2 is the electrostatic capacity of the second auxiliary capacitor 44b. The magnitude of Vd2 is set not to exceed the magnitude of a threshold voltage of a body diode of the third switch S3. In other words, the electrostatic capacity (Cg2) of the second auxiliary capacitor 44b and the first threshold (Vth) are set such that | Vd2|≤| Vf| is established.

In the second state, the constant output current I1 is supplied to the second electrode 32 from the power supply Iin. As a result, the capacitor 30 is charged such that the potential V2 of the second electrode 32 is higher than the potential V1 of the first electrode 31. In addition, charges are also accumulated in the second auxiliary capacitor 44b.

At time t32, the determination circuit 431 determines that the potential V2 of the second electrode 32 reaches the second threshold (Vth). As a result, the drive circuit 432 sets the charge and discharge circuit 42 to the first state. Immediately after the charge and discharge circuit 42 is switched to the first state, the potential V1 of the first electrode 31 is negative. Since the charge and discharge circuit 42 has the first auxiliary capacitor 44a connected in parallel with the first electrode 31, the first auxiliary capacitor 44a is connected in parallel with the capacitor 30 in the first state. As a result, the charges of the capacitor 30 move to the first auxiliary capacitor 44a. In FIG. 15, the potential V2 of the second electrode 32 decreases to Vd1. Vd1 is a negative value. Since Vd1 is determined by the charges stored in the capacitor 30 in the second state and the combined electrostatic capacity of the capacitor 30 and the first auxiliary capacitor 44a, the following Equation (7) is established.

$$\begin{matrix}[ Math . 7 ] & \\ \vert Vd 1 \vert = \frac{Ce}{Ce + Cg 1} \cdot \vert Vth \vert & ( 7 )\end{matrix}$$

In Equation (7), Cg1 is the electrostatic capacity of the first auxiliary capacitor 44a. The magnitude of Vd1 is set not to exceed the magnitude of a threshold voltage of a body diode of the fourth switch S4. In other words, the electrostatic capacity (Cg1) of the first auxiliary capacitor 44a and the second threshold (Vth) are set such that ∥Vd1|≤|Vf| is established.

In the present embodiment, values of the first threshold and the second threshold are equal, and are Vth. In Equation (6), Vth is the value of the first threshold, and in Equation (7), Vth is the value of the second threshold. In a case where the first threshold and the second threshold are different, Equation (6) is the following Equation (6a), and Equation (7) is the following Equation (7a).

$$\begin{matrix}[ Math . 8 ] & \\ \vert Vd 2 \vert = \frac{C e}{Ce + Cg 2} \cdot \vert Vth 1 \vert & ( 6 a )\end{matrix}$$

$$\vert Vd 1 \vert = \frac{C e}{C e + Cg 1} \cdot \vert Vth 2 \vert \qquad ( 7 a )$$

In the present embodiment, the threshold voltage of the body diode of the third switch S3 and the threshold voltage of the body diode of the fourth switch S4 are equal. In the above conditional expression of |Vd2|≤|Vf|, Vf is a lower limit value of the potential V2 of the second electrode 32 when the charge and discharge circuit 42 in a case where there is not the second auxiliary capacitor 44b is switched from the first state to the second state, and corresponds to the threshold voltage of the body diode of the third switch S3. In the above conditional expression of |Vd1|≤|Vf|, Vf is a lower limit value of the potential V1 of the first electrode 31 when the charge and discharge circuit 42 in a case where there is not the first auxiliary capacitor 44a is switched from the second state to the first state, and corresponds to the threshold voltage of the body diode of the fourth switch S4. When the magnitude of the threshold voltage of the body diode of the third switch S3 is Vf1 and the magnitude of the threshold voltage of the body diode of the fourth switch S4 is Vf2 and these magnitudes are distinguished from each other, the conditional expression of |Vd1|≥|Vf| is represented by the following Equation (8) and the conditional expression of |Vd2|≤|Vf| is represented by the following Equation (9).

$$\begin{matrix}[ Math . 9 ] & \\ \frac{C e}{C e + C g 1} \cdot \vert Vth 2 \vert \leq \vert Vf 2 \vert & ( 8 )\end{matrix}$$

$$\frac{C e}{C e + Cg 2} \cdot \vert Vth 1 \vert \leq \vert Vf 1 \vert \qquad ( 9 )$$

In the present embodiment, the electrostatic capacity of the first auxiliary capacitor 44a, the electrostatic capacity of the second auxiliary capacitor 44b, the first threshold, and the second threshold are set to satisfy the above Equations (8) and (9).

In the first state, the constant output current I1 is supplied from the power supply Iin to the first electrode 31. As a result, the capacitor 30 is charged such that the potential V1 of the first electrode 31 is higher than the potential V2 of the second electrode 32. In addition, charges are also accumulated in the first auxiliary capacitor 44a.

At times t33 and t35 in FIG. 15, the determination circuit 431 determines that the potential V1 of the first electrode 31 reaches the first threshold (Vth). As a result, the drive circuit 432 sets the charge and discharge circuit 42 to the second state. As a result, the capacitor 30 is charged such that the potential V2 of the second electrode 32 is higher than the potential V1 of the first electrode 31.

At time t34 in FIG. 6, the determination circuit 431 determines that the potential V2 of the second electrode 32 reaches the second threshold (Vth). As a result, the drive circuit 432 sets the charge and discharge circuit 42 to the first state. As a result, the capacitor 30 is charged such that the potential V1 of the first electrode 31 is higher than the potential V2 of the second electrode 32.

In FIG. 15, T indicates a period of charging and discharging of the capacitor 30. The period T is the sum of a first period T1 and a second period T2. The first period T1 is a time taken for the potential of the first electrode 31 to be from Vd1 to the first threshold (Vth) by supplying the constant output current I1 from the power supply Iin to the combined capacitor of the capacitor 30 and the first auxiliary capacitor 44a. The second period T2 is a time taken for the potential of the second electrode 32 to be from Vd2 to the second threshold (Vth) by supplying the constant output current I1 from the power supply Iin to the combined capacitor of the capacitor 30 and the second auxiliary capacitor 44b. Accordingly, the period T is given by the following Equation (10). In the following Equation (10), i is the value (current value) of the output current I1.

$$\begin{matrix}[ Math . 10 ] & \\ T = T 1 + T 2 = \frac{( Vth + Vd 1 ) \cdot ( Ce + Cg 1 )}{i} + \frac{( Vth + Vd 2 ) \cdot ( Ce + Cg 2 )}{i} & ( 10 )\end{matrix}$$

When Vd2 of the above Equation (6) and Vd1 of the above Equation (7) are substituted into Equation (10), the following Equation (11) is obtained.

$$\begin{matrix}[ Math . 11 ] & \\ T = \frac{Vth}{i} \cdot ( 4 Ce + Cg 1 + Cg 2 ) & ( 11 )\end{matrix}$$

As is clear from the above Equation (11), it is possible to calculate the electrostatic capacity Ce of the capacitor 30 from the period T.

Note that, in a case where the first threshold is Vth1 and the second threshold is Vth2, the above Equation (11) is converted to the following Equation (12).

$$\begin{matrix}[ Math . 12 ] & \\ T = \frac{( 2 Ce + Cg 1 ) \cdot Vth 1 + ( 2 Ce + Cg 2 ) \cdot Vth 2}{i} & ( 12 )\end{matrix}$$

As is clear from the above Equation (12), it is possible to calculate the electrostatic capacity Ce of the capacitor 30 from the period T. For simplification of the electrostatic capacity sensor 1, it is preferable that the first threshold and the second threshold are equal and the electrostatic capacity of the first auxiliary capacitor 44a and the electrostatic capacity of the second auxiliary capacitor 44b are equal.

In addition, it is possible to calculate the electrostatic capacity of the first auxiliary capacitor 44a and the electrostatic capacity of the second auxiliary capacitor 44b from the periods of time of the periods T1 and T2 constituting the period T. When T1≠T2, the electrostatic capacity of the first auxiliary capacitor 44a and the electrostatic capacity of the second auxiliary capacitor 44b are different. Then, values of the potential Vd1 in the period T1 and the potential Vd2 in the period T2 are also different. As described above, it is possible to calculate the electrostatic capacity of the first auxiliary capacitor 44a and the electrostatic capacity of the second auxiliary capacitor 44b based on the potentials Vd1 and Vd2 as well as the periods T1 and T2.

### 1.1.4 Effects and the Like

As described above, the electrostatic capacity sensor 1 includes the sensor unit 3 having the first electrode 31 and the second electrode 32 constituting the capacitor 30, and the electrostatic capacity detection circuit 4 connected to the sensor unit 3. The electrostatic capacity detection circuit 4 includes the charge and discharge circuit 42 that is connected to the first electrode 31 and the second electrode 32 to charge and discharge the capacitor 30, the control circuit 43 that controls the charge and discharge circuit 42 such that the capacitor 30 repeats charge and discharge, and the auxiliary capacity circuit 44 that includes the first auxiliary capacitor 44a connected to the first electrode 31 in parallel with the capacitor 30 and the second auxiliary capacitor 44b connected to the second electrode 32 in parallel with the capacitor 30. This configuration can reduce the influence of the stray capacitance on the detection of the electrostatic capacity.

In the electrostatic capacity sensor 1, the electrostatic capacity of the first auxiliary capacitor 44a and the electrostatic capacity of the second auxiliary capacitor 44b are equal. This configuration can reduce the influence of the stray capacitance on the detection of the electrostatic capacity.

In the electrostatic capacity sensor 1, the electrostatic capacity of the first auxiliary capacitor 44a and the electrostatic capacity of the second auxiliary capacitor 44b are different. This configuration can reduce the influence of the stray capacitance on the detection of the electrostatic capacity.

In the electrostatic capacity sensor 1, the charge and discharge circuit 42 is configured to be complementary switchable in the first state where the constant output current is supplied to the first electrode 31 and the second state where the constant output current is supplied to the second electrode 32. The control circuit 43 is configured to switch the charge and discharge circuit 42 from the first state to the second state when the potential of the first electrode 31 reaches the first threshold in a case where the charge and discharge circuit 42 is in the first state. The control circuit 43 is configured to switch the charge and discharge circuit 42 from the second state to the first state when the potential of the second electrode 32 reaches the second threshold in a case where the charge and discharge circuit 42 is in the second state. With this configuration, the configuration of the electrostatic capacity detection circuit can be simplified.

In the electrostatic capacity sensor 1, the first threshold and the second threshold are equal. With this configuration, the configuration of the electrostatic capacity detection circuit can be simplified.

In the electrostatic capacity sensor 1, the charge and discharge circuit 42 is connected between the power supply terminal 41a connected to the power supply Iin and the reference potential terminal 41b connected to the reference potential Vg, and includes the first switch S1, the second switch S2, the third switch S3, and the fourth switch S4. The first switch S1 and the third switch S3 constitute the series circuit. The series circuit of the first switch S1 and the third switch S3 is present between the power supply terminal 41a and the reference potential terminal 41b such that the first switch S1 is connected to the power supply terminal 41a and the third switch S3 is connected to the reference potential terminal 41b. The connection point of the first switch S1 and the third switch S3 is connected to the first electrode 31. The second switch S2 and the fourth switch S4 constitute the series circuit. The series circuit of the second switch S2 and the fourth switch S4 is present between the power supply terminal 41a and the reference potential terminal 41b such that the second switch S2 is connected to the power supply terminal 41a and the fourth switch S4 is connected to the reference potential terminal 41b, and is connected in parallel with the series circuit of the first switch S1 and the third switch S3. The connection point of the second switch S2 and the fourth switch S4 is connected to the second electrode 32. In the first state, the first switch S1 and the fourth switch S4 are turned on, and the second switch S2 and the third switch S3 are turned off. In the second state, the first and fourth switches S4 are turned off, and the second and third switches S3 are turned on. With this configuration, the configuration of the electrostatic capacity detection circuit can be simplified.

In the electrostatic capacity sensor 1, the first end of the first auxiliary capacitor 44a is connected to the first electrode 31 and the second end of the first auxiliary capacitor 44a is connected to the reference potential terminal 41b such that the first auxiliary capacitor 44a is in parallel with the third switch S3. The first end of the second auxiliary capacitor 44b is connected to the second electrode 32 and the second end of the second auxiliary capacitor 44b is connected to the reference potential terminal 41b such that the second auxiliary capacitor 44b is in parallel with the fourth switch S4. This configuration can reduce the influence of the stray capacitance on the detection of the electrostatic capacity.

In the electrostatic capacity sensor 1, the following equation is satisfied.

$$\begin{matrix}[ Math . 13 ] & \\ \frac{Ce}{Ce + Cg 1} \cdot \vert Vth 2 \vert \leq \vert Vf 2 \vert & \end{matrix}$$

$$\frac{Ce}{Ce + Cg 2} \cdot \vert Vth 1 \vert \leq \vert Vf 1 \vert$$

Ce is the electrostatic capacity of the capacitor 30. Cg1 is the electrostatic capacity of the first auxiliary capacitor 44a. Cg2 is the electrostatic capacity of the second auxiliary capacitor 44b. Vth1 is the first threshold. Vth2 is the second threshold. Vf1 is a lower limit value of the potential of the first electrode 31 when the charge and discharge circuit 42 in a case where there is not the first auxiliary capacitor 44a is switched from the second state to the first state. Vf2 is a lower limit value of the potential of the second electrode 32 when the charge and discharge circuit 42 in a case where there is not the second auxiliary capacitor 44b is switched from the first state to the second state. This configuration can reduce the influence of the stray capacitance on the detection of the electrostatic capacity.

Note that, in a case where the electrostatic capacity of the first auxiliary capacitor 44a and the electrostatic capacity of the second auxiliary capacitor 44b are equal, Cg1=Cg2=Cg can be obtained. In a case where the first threshold and the second threshold are equal, Vth1=Vth2=Vth can be obtained. In a case where the lower limit value of the potential of the first electrode 31 when the charge and discharge circuit 42 without the first auxiliary capacitor 44a is switched from the second state to the first state and the lower limit value of the potential of the second electrode 32 when the charge and discharge circuit 42 without the second auxiliary capacitor 44b is switched from the first state to the second state are equal, Vf1=Vf2=Vf can be obtained. In this case, the electrostatic capacity detection circuit 4 may satisfy the following equation.

$$\begin{matrix}[ Math . 14 ] & \\ \frac{C e}{Ce + Cg} \cdot \vert Vth \vert \leq \vert Vf \vert & \end{matrix}$$

In the electrostatic capacity sensor 1, Vf1=Vf2 is satisfied. With this configuration, the configuration of the electrostatic capacity detection circuit can be simplified.

In the electrostatic capacity sensor 1, Vf1<0 and Vf2<0 are satisfied. This configuration can increase the amount of change in the electrostatic capacity, and can improve the detection accuracy of the electrostatic capacity.

In the electrostatic capacity sensor 1, the third switch S3 and the fourth switch S4 are the field effect transistors. Vf1 is determined by the threshold voltage of the body diode of the second switch S3. Vf2 is determined by the threshold voltage of the body diode of the fourth switch S4. This configuration can reduce a size of the electrostatic capacity detection circuit, and can increase a speed of switching between the first state and the second state.

The electrostatic capacity sensor 1 further includes the processing circuit 5 that calculates the electrostatic capacity of the capacitor 30 based on the charging and discharging time of the capacitor 30 by the electrostatic capacity detection circuit 4. This configuration can reduce the influence of the stray capacitance on the detection of the electrostatic capacity.

In the electrostatic capacity sensor 1, the sensor unit 3 has the sensor substrate 33 on which the first electrode 31 and the second electrode 32 are disposed. The charge and discharge circuit 42 is disposed on the circuit substrate 4a different from the sensor substrate 33. The auxiliary capacity circuit 44 is disposed between the sensor substrate 33 and the circuit substrate 4a and at a position closer to the circuit substrate 4a than the sensor substrate 33. This configuration can reduce the influence of stray capacitance from the first electrode 31 and the second electrode 32 of the sensor unit 3.

The measuring instrument 10 described above includes the electrostatic capacity sensor 1 and the handheld housing 2 that accommodates the electrostatic capacity sensor 1. This configuration can reduce the influence of the stray capacitance on the detection of the electrostatic capacity.

In the measuring instrument 10, the handheld housing 2 includes the head portion 21 that is disposed at the first end of the handheld housing 2 and comes into contact with the measurement target, the grip portion 22 that is disposed at the second end of the handheld housing 2 and is gripped with hand, and the probe portion 23 that couples the head portion 21 and the grip portion 22. The sensor unit 3 is positioned in the head portion 21. The electrostatic capacity detection circuit 4 is positioned in the head portion 21 or the probe portion 23. The processing circuit 5 is positioned in the grip portion 22. This configuration can reduce the influence of the stray capacitance generated in the grip portion.

In the measuring instrument 10, the grip portion 22 has the conductive portion 221 exposed on the surface of the grip portion 22. The conductive portion 221 is connected to a reference potential Vg of the processing circuit 5. This configuration can reduce the variation in the influence of the stray capacitance on the side of the person who has the measuring instrument.

In the measuring instrument 10, the sensor unit 3 is formed such that the first and second electrodes 31 and 32 form the capacitor 30 together with a part of the measurement target by bringing the first and second electrodes 31 and 32 into contact with the measurement target. The processing circuit 5 is configured to obtain the moisture content of the measurement target based on the electrostatic capacity of the capacitor 30. This configuration can measure the moisture content of the measurement target.

In the measuring instrument 10, the measurement target is the organism. This configuration can measure the moisture content of the organism.

In the measuring instrument 10, the measurement target is the oral cavity of the organism. This configuration can measure the moisture content in the oral cavity of the organism.

### 1.2 Second Embodiment

### 1.2.1 Configuration

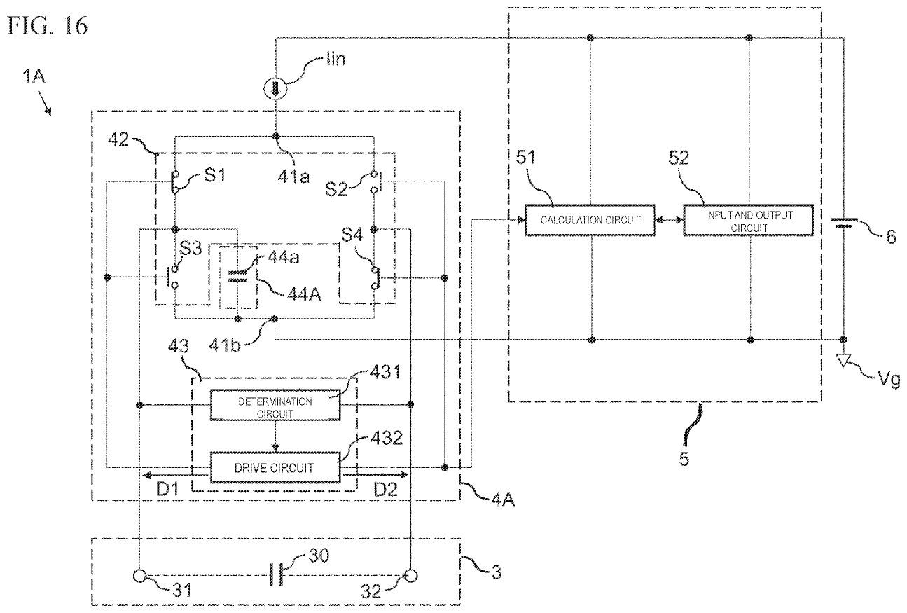

FIG. 16 is a circuit diagram of a configuration example of an electrostatic capacity sensor 1A of a measuring instrument according to a second embodiment. The electrostatic capacity sensor 1A is different from the electrostatic capacity sensor 1 in that an electrostatic capacity detection circuit 4A different from the electrostatic capacity detection circuit 4 of the electrostatic capacity sensor 1 is included. The electrostatic capacity detection circuit 4A in FIG. 16 is different from the electrostatic capacity detection circuit 4 in that an auxiliary capacity circuit 44A different from the auxiliary capacity circuit 44 of the electrostatic capacity detection circuit 4 is included. The auxiliary capacity circuit 44A is different from the auxiliary capacity circuit 44 in that the first auxiliary capacitor 44a is included but a second auxiliary capacitor 44b is not included.

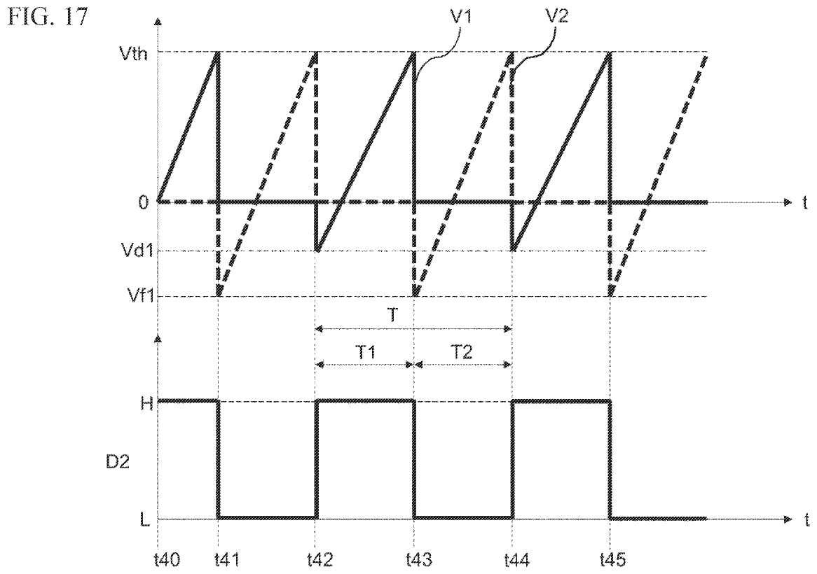

Next, an example of an operation of the electrostatic capacity detection circuit 4A will be described with reference to FIGS. 17 to 23.

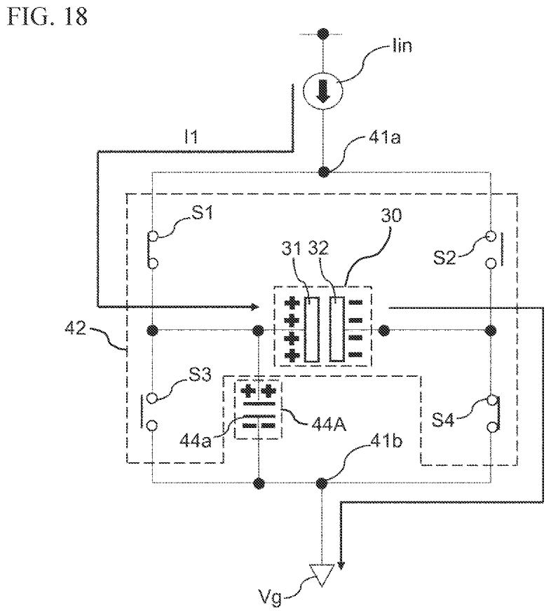

FIG. 17 is a timing chart of an example of the operation of the electrostatic capacity detection circuit 4A. In FIG. 17, V1 indicates a potential of a first electrode 31, and V2 indicates a potential of a second electrode 32. In FIG. 17, H corresponds to a state where the voltage value of the second drive signal D2 is at the high level, and L indicates a state where the voltage value of the second drive signal D2 is at the low level. FIGS. 18 to 23 are explanatory diagrams of an example of the operation of the electrostatic capacity detection circuit 4A. In FIGS. 18 to 23, the control circuit 43 is omitted only for simplification of the drawings.

At time t40 in FIG. 17, charges are not accumulated in the capacitor 30. The drive circuit 432 sets the charge and discharge circuit 42 to the first state by setting the voltage value of the first drive signal D1 to the high level and the voltage value of the second drive signal D2 to the low level.

FIG. 18 is an explanatory diagram of the operation of the electrostatic capacity detection circuit 4A when the charge and discharge circuit 42 is in the first state. As illustrated in FIG. 18, in the first state, the first and fourth switches S1 and S4 are turned on, and the second and third switches S2 and S3 are turned off. The constant output current I1 is supplied from the power supply Iin to the first electrode 31. As a result, the capacitor 30 is charged such that the potential V1 of the first electrode 31 is higher than the potential V2 of the second electrode 32. Since the charge and discharge circuit 42 has the first auxiliary capacitor 44a connected in parallel with the first electrode 31, the first auxiliary capacitor 44a is connected in parallel with the capacitor 30 in the first state, and charges are also accumulated in the first auxiliary capacitor 44a.

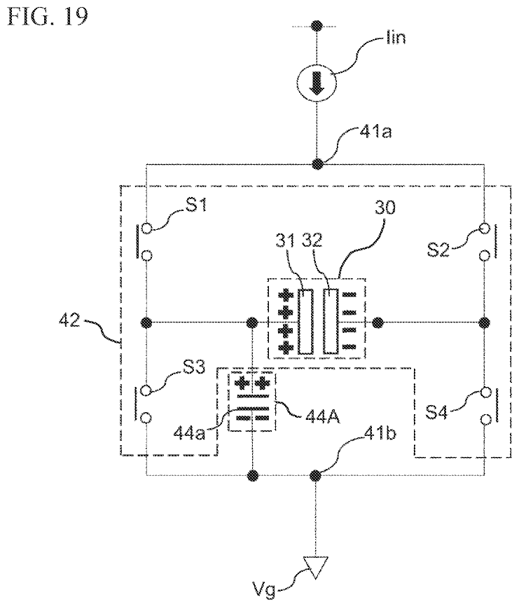

The determination circuit 431 executes determination as to whether or not the potential V1 of the first electrode 31 reaches the first threshold in a case where the charge and discharge circuit 42 is in the first state. In FIG. 17, the first threshold is Vth. At time t41, the determination circuit 431 determines that the potential V1 of the first electrode 31 reaches the first threshold (Vth). As a result, the drive circuit 432 sets the charge and discharge circuit 42 to the second state. In the present embodiment, the drive circuit 432 sets the charge and discharge circuit 42 to the third state before setting the charge and discharge circuit 42 to the second state. FIG. 19 is an explanatory diagram of the operation of the electrostatic capacity detection circuit 4A when the charge and discharge circuit 42 is in the third state. Thereafter, the drive circuit 432 sets the charge and discharge circuit 42 to the second state by setting the voltage value of the first drive signal D1 to the low level and the voltage value of the second drive signal D2 to the high level.

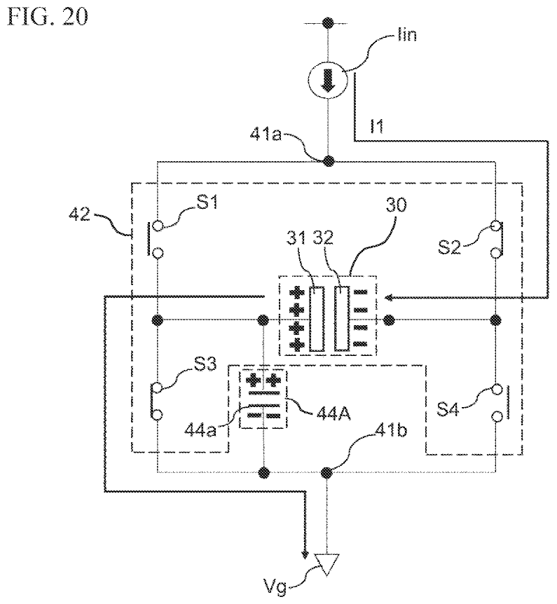

FIG. 20 is an explanatory diagram of the operation of the electrostatic capacity detection circuit 4A immediately after the charge and discharge circuit 42 is switched to the second state. As illustrated in FIG. 20, in the second state, the first and fourth switches S1 and S4 are turned off, and the second and third switches S2 and S3 are turned on. In the capacitor 30, the first electrode 31 is connected to the reference potential terminal 41b, and the second electrode 32 is connected to the power supply terminal 41a. Immediately after the charge and discharge circuit 42 is switched to the second state, the potential V2 of the second electrode 32 is negative. The charge and discharge circuit 42 does not have the second auxiliary capacitor 44b connected in parallel with the second electrode 32. Thus, unlike the first embodiment, the potential V2 of the second electrode 32 decreases to Vf1 in FIG. 17. Vf1 is a negative value, and the magnitude of Vf1 is equal to a threshold voltage of a body diode of a field effect transistor used as the third switch S3.

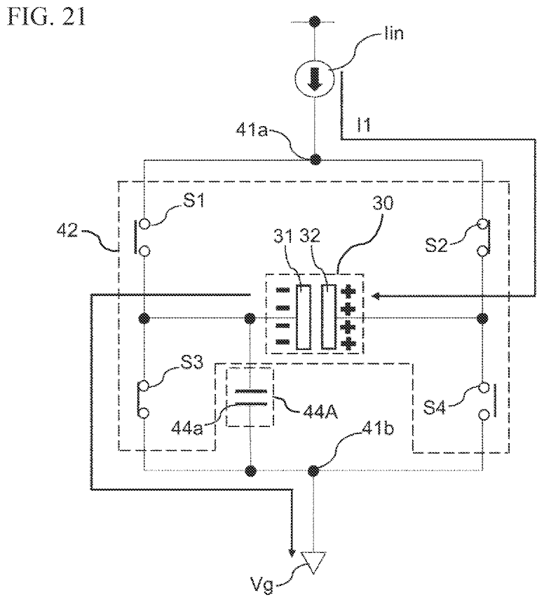

In the second state, the constant output current I1 is supplied to the second electrode 32 from the power supply Iin. As a result, the capacitor 30 is charged such that the potential V2 of the second electrode 32 is higher than the potential V1 of the first electrode 31. FIG. 21 is an explanatory diagram of the operation of the electrostatic capacity detection circuit 4A when a time elapses since the charge and discharge circuit 42 is switched to the second state. In FIG. 21, the potential V2 of the second electrode 32 is positive.

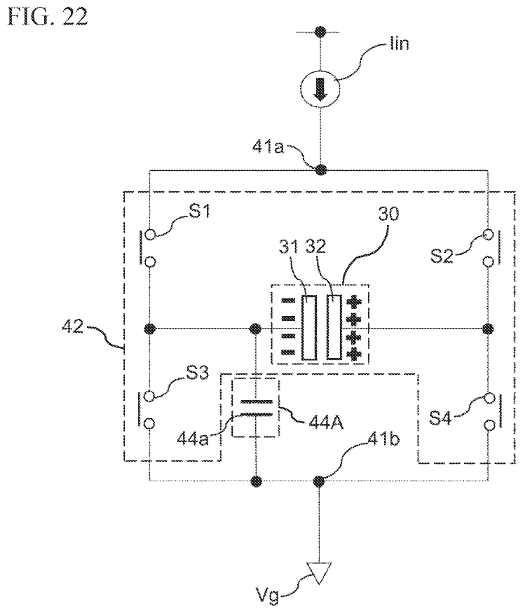

The determination circuit 431 executes determination as to whether or not the potential V2 of the second electrode 32 reaches the second threshold in a case where the charge and discharge circuit 42 is in the second state. In FIG. 17, the second threshold is equal to the first threshold and is Vth. At time t42, the determination circuit 431 determines that the potential V2 of the second electrode 32 reaches the second threshold (Vth). As a result, the drive circuit 432 sets the charge and discharge circuit 42 to the first state. In the present embodiment, the drive circuit 432 sets the charge and discharge circuit 42 to the third state before setting the charge and discharge circuit 42 to the first state. FIG. 22 is an explanatory diagram of the operation of the electrostatic capacity detection circuit 4A when the charge and discharge circuit 42 is in the third state. Thereafter, the drive circuit 432 sets the charge and discharge circuit 42 to the first state by setting the voltage value of the first drive signal D1 to the high level and the voltage value of the second drive signal D2 to the low level.

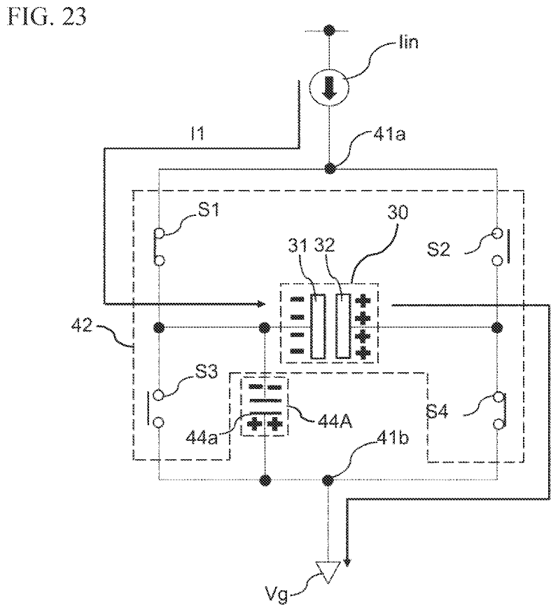

FIG. 23 is an explanatory diagram of the operation of the electrostatic capacity detection circuit 4A immediately after the charge and discharge circuit 42 is switched to the first state. As illustrated in FIG. 23, in the first state, the first and fourth switches S1 and S4 are turned on, and the second and third switches S2 and S3 are turned off. In the capacitor 30, the first electrode 31 is connected to the power supply terminal 41a, and the second electrode 32 is connected to the reference potential terminal 41b. Immediately after the charge and discharge circuit 42 is switched to the first state, the potential V1 of the first electrode 31 is negative. Since the charge and discharge circuit 42 has the first auxiliary capacitor 44a connected in parallel with the first electrode 31, the first auxiliary capacitor 44a is connected in parallel with the capacitor 30 in the first state. As a result, the charges of the capacitor 30 move to the first auxiliary capacitor 44a. In FIG. 17, the potential V1 of the first electrode 31 decreases to Vd1. Vd1 is a negative value. Since Vd1 is determined by the charges stored in the capacitor 30 in the second state and the combined electrostatic capacity of the capacitor 30 and the first auxiliary capacitor 44a, the above Equation (7) is established.

In the present embodiment, the electrostatic capacity (Cg1) of the first auxiliary capacitor 44a and the second threshold (Vth) are set such that | Vd1|≤|Vf2| is established. Vf2 is a negative value, and the magnitude of Vf2 is equal to the threshold voltage of the body diode of the field effect transistor used as the fourth switch S4. In the case of |Vd1|>|Vf21, since the forward voltage of the body diode of the fourth switch S4 exceeds |Vf2|, the potential V1 of the first electrode 31 decreases to Vf2.

In the first state, the constant output current I1 is supplied from the power supply Iin to the first electrode 31. As a result, the capacitor 30 is charged such that the potential V1 of the first electrode 31 is higher than the potential V2 of the second electrode 32. In addition, charges are also accumulated in the first auxiliary capacitor 44a. After a time elapses since the charge and discharge circuit 42 is switched to the first state, the potential V1 of the first electrode 31 is positive as illustrated in FIG. 18.

At times t43 and t45 in FIG. 17, the determination circuit 431 determines that the potential V1 of the first electrode 31 reaches the first threshold (Vth). As a result, the drive circuit 432 sets the charge and discharge circuit 42 to the second state. As a result, the capacitor 30 is charged such that the potential V2 of the second electrode 32 is higher than the potential V1 of the first electrode 31.

At time t44 in FIG. 17, the determination circuit 431 determines that the potential V2 of the second electrode 32 reaches the second threshold (Vth). As a result, the drive circuit 432 sets the charge and discharge circuit 42 to the first state. As a result, the capacitor 30 is charged such that the potential V1 of the first electrode 31 is higher than the potential V2 of the second electrode 32.

In FIG. 17, T indicates a period of charging and discharging of the capacitor 30. The period T is the sum of a first period T1 and a second period T2. The first period T1 is a time taken for the potential of the first electrode 31 to be from Vd1 to the first threshold (Vth) by supplying the constant output current I1 from the power supply Iin to the combined capacitor of the capacitor 30 and the first auxiliary capacitor 44a. The second period T2 is a time taken for the potential of the second electrode 32 to be from Vf1 to the second threshold (Vth) by supplying the constant output current I1 from the power supply Iin to the capacitor 30. Accordingly, the period T is given by the following Equation (12). In the following Equation (12), i is a value (current value) of the output current I1.

$$\begin{matrix}[ Math . 15 ] & \\ T = T 1 + T 2 = \frac{( Vth + Vd 1 ) \cdot ( Ce + Cg 1 )}{i} + \frac{( Vth + Vf 1 ) \cdot Ce}{i} & ( 13 )\end{matrix}$$

When Vd1 of the above Equation (7) is substituted into Equation (13), the following Equation (13) is obtained.

$$\begin{matrix}[ Math . 16 ] & \\ T = \frac{Vth}{i} \cdot ( 2 Ce + Cg 1 ) + \frac{Vth + Vf 1}{i} \cdot Ce & ( 14 )\end{matrix}$$

As is clear from the above Equation (14), it is possible to calculate the electrostatic capacity Ce of the capacitor 30 from the period T.

### 1.2.2 Modification Example

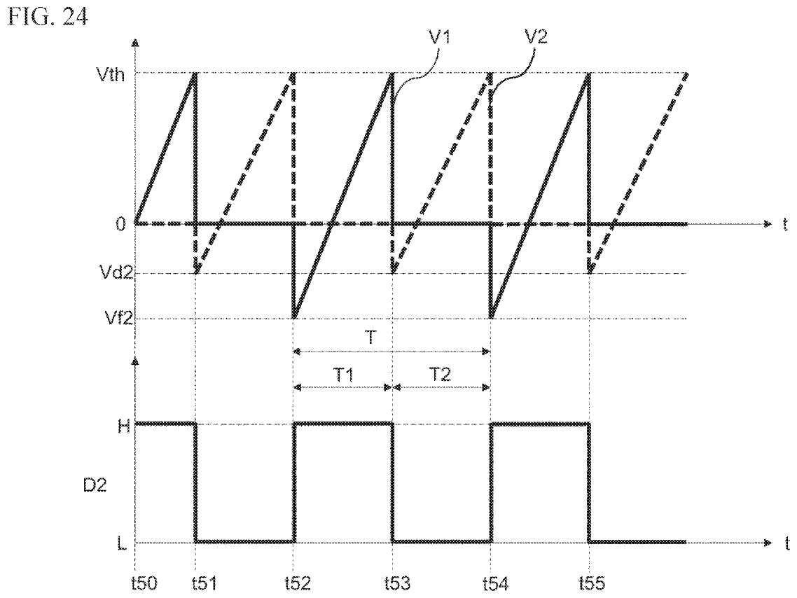

FIG. 24 is a timing chart of an example of an operation of an electrostatic capacity detection circuit according to a modification example of the second embodiment. In the present modification example, the electrostatic capacity detection circuit is different from the electrostatic capacity detection circuit 4A in that the second auxiliary capacitor 44b is included but the first auxiliary capacitor 44a is not included.

At time t50 in FIG. 24, charges are not accumulated in the capacitor 30. The drive circuit 432 sets the charge and discharge circuit 42 to the first state by setting the voltage value of the first drive signal D1 to the high level and the voltage value of the second drive signal D2 to the low level.

In the first state, the constant output current I1 is supplied from the power supply Iin to the first electrode 31. As a result, the capacitor 30 is charged such that the potential V1 of the first electrode 31 is higher than the potential V2 of the second electrode 32.

At time t51, the determination circuit 431 determines that the potential V1 of the first electrode 31 reaches the first threshold (Vth). As a result, the drive circuit 432 sets the charge and discharge circuit 42 to the second state. Immediately after the charge and discharge circuit 42 is switched to the second state, the potential V2 of the second electrode 32 is negative. Since the charge and discharge circuit 42 has the second auxiliary capacitor 44b connected in parallel with the second electrode 32, the second auxiliary capacitor 44b is connected in parallel with the capacitor 30 in the second state. As a result, the charges of the capacitor 30 move to the second auxiliary capacitor 44b. In FIG. 24, the potential V2 of the second electrode 32 decreases to Vd2. Vd2 is a negative value. Since Vd2 is determined by the charges stored in the capacitor 30 in the first state and the combined electrostatic capacity of the capacitor 30 and the second auxiliary capacitor 44b, the above Equation (6) is established.

In the present modification example, the electrostatic capacity (Cg2) of the second auxiliary capacitor 44b and the first threshold (Vth) are set such that |Vd2|≤|Vf1| is established. Vf1 is a negative value, and the magnitude of Vf1 is equal to a threshold voltage of a body diode of a field effect transistor used as the third switch S3. In the case of |Vd2|>|Vf1|, since the forward voltage of the body diode of the third switch S3 exceeds |Vf1|, the potential V2 of the second electrode 32 decreases to Vf1.

In the second state, the constant output current I1 is supplied to the second electrode 32 from the power supply Iin. As a result, the capacitor 30 is charged such that the potential V2 of the second electrode 32 is higher than the potential V1 of the first electrode 31. In addition, charges are also accumulated in the second auxiliary capacitor 44b.

At time t52, the determination circuit 431 determines that the potential V2 of the second electrode 32 reaches the second threshold (Vth). As a result, the drive circuit 432 sets the charge and discharge circuit 42 to the first state. Immediately after the charge and discharge circuit 42 is switched to the first state, the potential V1 of the first electrode 31 is negative. In FIG. 24, the potential V1 of the first electrode 31 decreases to Vf2. Vf2 is a negative value. The magnitude of Vf2 is equal to the threshold voltage of the body diode of the field effect transistor used as the fourth switch S4.

In the first state, the constant output current I1 is supplied from the power supply Iin to the first electrode 31. As a result, the capacitor 30 is charged such that the potential V1 of the first electrode 31 is higher than the potential V2 of the second electrode 32.

At times t53 and t55 in FIG. 24, the determination circuit 431 determines that the potential V1 of the first electrode 31 reaches the first threshold (Vth). As a result, the drive circuit 432 sets the charge and discharge circuit 42 to the second state. As a result, the capacitor 30 is charged such that the potential V2 of the second electrode 32 is higher than the potential V1 of the first electrode 31.

At time t54 in FIG. 24, the determination circuit 431 determines that the potential V2 of the second electrode 32 reaches the second threshold (Vth). As a result, the drive circuit 432 sets the charge and discharge circuit 42 to the first state. As a result, the capacitor 30 is charged such that the potential V1 of the first electrode 31 is higher than the potential V2 of the second electrode 32.

In FIG. 24, T indicates a period of charging and discharging of the capacitor 30. The period T is the sum of a first period T1 and a second period T2. The first period T1 is a time taken for the potential of the first electrode 31 to be Vth from Vf2 by supplying the constant output current I1 from the power supply Iin to the capacitor 30. The second period T2 is a time taken for the potential of the second electrode 32 to be from Vd2 to Vth by supplying the constant output current I1 from the power supply Iin to the combined capacitor of the capacitor 30 and the second auxiliary capacitor 44b. Accordingly, the period T is given by the following Equation (14). In the following Equation (14), i is a value (current value) of the output current I1.

$$\begin{matrix}[ Math . 17 ] & \\ T = T 1 + T 2 = \frac{( Vth + Vf 2 ) \cdot Ce}{i} + \frac{( Vth + Vd 2 ) \cdot ( Ce + Cg 2 )}{i} & ( 15 )\end{matrix}$$

When Vd2 of the above Equation (6) is substituted into Equation (15), the following Equation (16) is obtained.

$$\begin{matrix}[ Math . 18 ] & \\ T = \frac{Vth + Vf 2}{i} \cdot Ce + \frac{Vth}{i} \cdot ( 2 Ce + Cg 2 ) & ( 16 )\end{matrix}$$

As is clear from the above Equation (16), it is possible to calculate the electrostatic capacity Ce of the capacitor 30 from the period T.

### 1.2.3 Effects and the Like

As described above, the electrostatic capacity sensor 1A includes the sensor unit 3 having the first electrode 31 and the second electrode 32 constituting the capacitor 30, and the electrostatic capacity detection circuit 4A connected to the sensor unit 3. The electrostatic capacity detection circuit 4A includes the charge and discharge circuit 42 connected to the first electrode 31 and the second electrode 32 to charge and discharge the capacitor 30, the control circuit 43 that controls the charge and discharge circuit 42 such that the capacitor 30 repeats charge and discharge, and the auxiliary capacity circuit 44A having one of the first auxiliary capacitor 44a connected to the first electrode 31 in parallel with the capacitor 30 and the second auxiliary capacitor 44b connected to the second electrode 32 in parallel with the capacitor 30. This configuration can reduce the influence of the stray capacitance on the detection of the electrostatic capacity.

### 1.3 Third Embodiment

### 1.3.1 Configuration

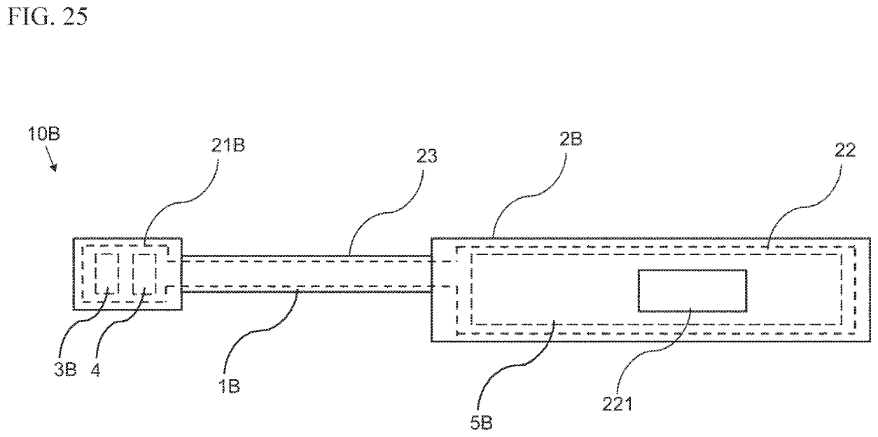

FIG. 25 is a schematic diagram of a configuration example of a measuring instrument 10B according to a third embodiment. The measuring instrument 10B is an occlusal force measuring instrument for measuring an occlusal force of upper and lower jaw teeth of a person.

The measuring instrument 10B in FIG. 25 is an electrostatic capacity type occlusal force measuring instrument. The measuring instrument 10B includes an electrostatic capacity sensor 1B and a handheld housing 2B.

The handheld housing 2B accommodates the electrostatic capacity sensor 1B. The handheld housing 2B has a size and a weight that can be held by a person with one hand. The handheld housing 2B has a waterproof structure and protects the electrostatic capacity sensor 1B within the handheld housing 2B from moisture. The handheld housing 2B in FIG. 25 has a rod shape. The handheld housing 2B in FIG. 25 has a shape like a so-called toothbrush. The handheld housing 2B includes a head portion 21B, the grip portion 22, and the probe portion 23 that couples the head portion 21B and the grip portion 22. The handheld housing 2B in FIG. 25 is different from the handheld housing 2 in FIG. 1 in the structure of the head portion 21B.

The head portion 21B is a part of the handheld housing 2B that comes into contact with the measurement target. The head portion 21B is disposed at a first end of the handheld housing 2B (left end in FIG. 25). In the present embodiment, the head portion 21B is placed in the human oral cavity when used and is sandwiched between the upper and lower jaw teeth. The head portion 21B is made of a soft material to transmit the occlusal force by the upper and lower jaw teeth to the electrostatic capacity sensor 1B.

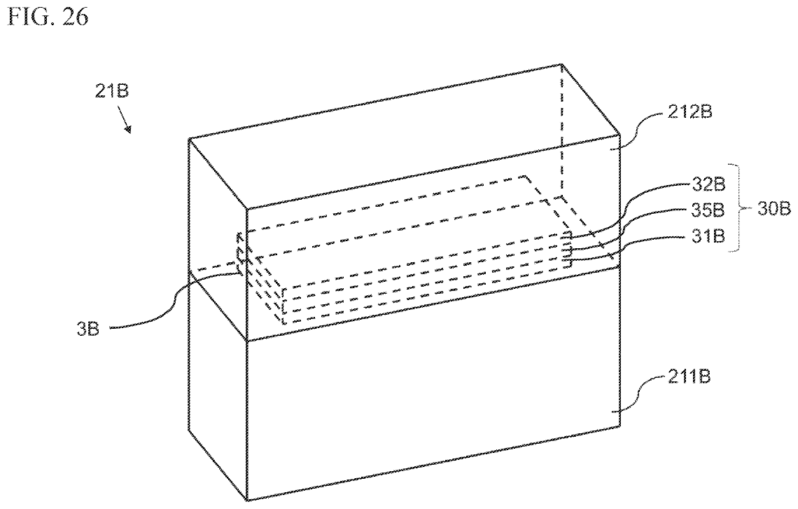

FIG. 26 is a schematic perspective view of a configuration example of the head portion 21B. The head portion 21B has a pair of resin layers 211B and 212B. A sensor unit 3B to be described later is disposed between the pair of resin layers 211B and 212B. The resin layers 211B and 212B have, for example, a rectangular plate shape. The resin layers 211B and 212B are made of a flexible resin. The flexible resin includes an acrylic resin, a urethane resin, a silicone resin, a styrene resin, and a polyamide resin.

The electrostatic capacity sensor 1B obtains the occlusal force based on the electrostatic capacity. The electrostatic capacity sensor 1B includes the sensor unit 3B, the electrostatic capacity detection circuit 4, and a processing circuit 5B. In the present embodiment, the sensor unit 3B and the electrostatic capacity detection circuit 4 are positioned in the head portion 21B of the handheld housing 2B. In the present embodiment, the processing circuit 5B is positioned in the grip portion 22B of the handheld housing 2B.

As illustrated in FIG. 26, the sensor unit 3B includes first and second electrodes 31B and 32B, and a deformation portion 35B. The deformation portion 35B is deformed by an applied pressure. The pressure can be applied, for example, by a person biting with the upper and lower jaw teeth. The deformation portion 35B has, for example, a rectangular plate shape. The deformation portion 35B is made of a flexible resin. The flexible resin includes an acrylic resin, a urethane resin, a silicone resin, a styrene resin, and a polyamide resin. These resins have a large change in physical properties with respect to a load, and it is possible to suppress a load on the user. The first and second electrodes 31B and 32B have, for example, a rectangular plate shape. The first and second electrodes 31B and 32B can be formed by sputtering, evaporation, or printing. A precious metal such as Au, Ag, and Pd, and a base metal such as Cu, Al, and Ni are included as materials of the first and second electrodes 31B and 32B. In the sensor unit 3B, the deformation portion 35B is present between the first and second electrodes 31B and 32B. As a result, the sensor unit 3B is formed such that the first and second electrodes 31B and 32B form the capacitor 30B together with the deformation portion 35B. More specifically, the first and second electrodes 31B and 32B function as electrodes of the capacitor 30B. The deformation portion 35B functions as a dielectric for the first and second electrodes 31B and 32B. In other words, the deformation portion 35B having flexibility is displaced, and thus, the electrostatic capacity between the first and second electrodes 31B and 32B, that is, the electrostatic capacity of the capacitor 30B changes.

The processing circuit 5B is different from the processing circuit 5 in FIG. 2 in the operation of the calculation circuit 51. In a case where the calculation circuit 51 performs an operation to start measuring the occlusal force by the input device of the input and output circuit 52, the calculation circuit 51 of the processing circuit 5B of FIG. 25 causes the electrostatic capacity detection circuit 4 to start the operation for detecting the electrostatic capacity. The calculation circuit 51 is configured to calculate the electrostatic capacity of the capacitor 30 based on a charging and discharging time of the capacitor 30 by the electrostatic capacity detection circuit 4. As with the first and second embodiments, the calculation circuit 51 can obtain the electrostatic capacity Ce of the capacitor 30B from the period T. The calculation circuit 51 is configured to obtain the occlusal force of the upper and lower jaw teeth based on the electrostatic capacity Ce of the capacitor 30B. The calculation circuit 51 displays information indicating the occlusal force by the output device of the input and output circuit 52.

### 1.3.2 Effects and the Like

In the measuring instrument 10B described above, the sensor unit 3B includes the deformation portion 35B deformed by the applied pressure. The sensor unit 3B is formed such that the first and second electrodes 31B and 32B form the capacitor 30B together with the deformation portion 35B. The processing circuit 5B is configured to obtain the pressure based on the electrostatic capacity of the capacitor 30B. This configuration can measure the pressure.

In particular, the pressure may be applied to the deformation portion 35B by a person biting with the upper and lower jaw teeth. In this case, it is possible to measure the occlusal force of the upper and lower jaw teeth of a person.

### 1.4 Fourth Embodiment

### 1.4.1 Configuration

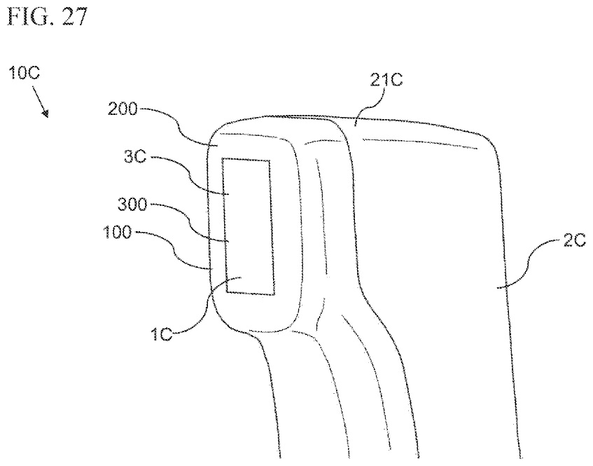

FIG. 27 is a schematic perspective view of a configuration example of a head portion 21C of a measuring instrument 10C according to a fourth embodiment. As with the measuring instrument 10, the measuring instrument 10C is an electrostatic capacity type moisture measuring instrument. The measuring instrument 10C includes an electrostatic capacity sensor 1C and a handheld housing 2C.

The handheld housing 2C accommodates the electrostatic capacity sensor 1C. The handheld housing 2C includes a head portion 21C. Although not illustrated in FIG. 27, the handheld housing 2C includes the grip portion 22 and the probe portion 23, as with the handheld housing 2 in FIG. 1.

The electrostatic capacity sensor 1C calculates the moisture content of the measurement target based on the electrostatic capacity. The electrostatic capacity sensor 1C includes a sensor unit 3C. As with the electrostatic capacity sensor 1 of FIG. 2, the electrostatic capacity sensor 1C includes the electrostatic capacity detection circuit 4 and the processing circuit 5.

In the present embodiment, at least the sensor unit 3C is positioned in the head portion 21C of the handheld housing 2C. A surface 300 of the sensor unit 3C is exposed to an outside from the head portion 21C of the handheld housing 2C. The surface 300 of the sensor unit 3C and a frame-shaped region 200 surrounding the surface 300 of the sensor unit 3C in the head portion 21C constitute a contact region 100 that comes into contact with the measurement target. The contact region 100 is a region planned to come into contact with the measurement target at the time of measurement by the measuring instrument 10C. In the present embodiment, the surface 300 of the sensor unit 3C is positioned on a predetermined plane that includes the frame-shaped region 200 of the head portion 21C. In other words, it can be said that the surface 300 of the sensor unit 3C and the frame-shaped region 200 of the head portion 21C are on the same plane.

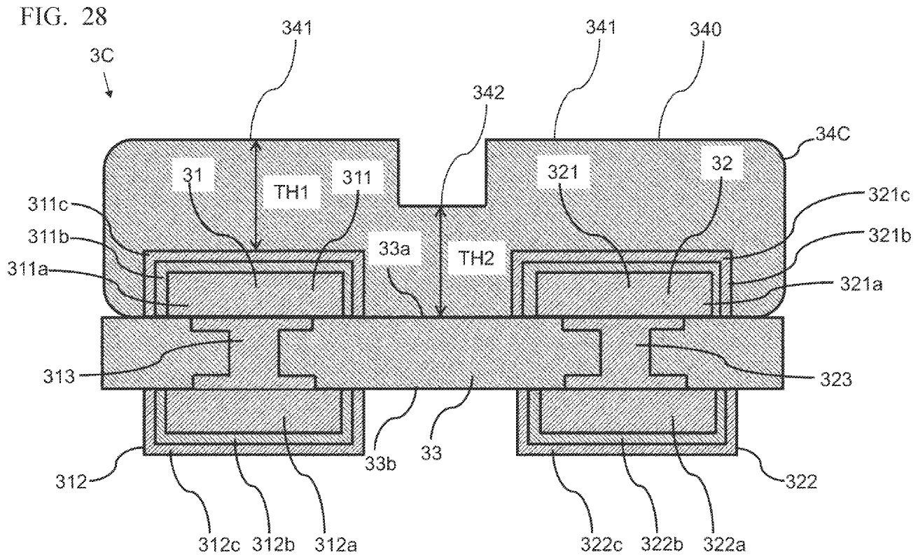

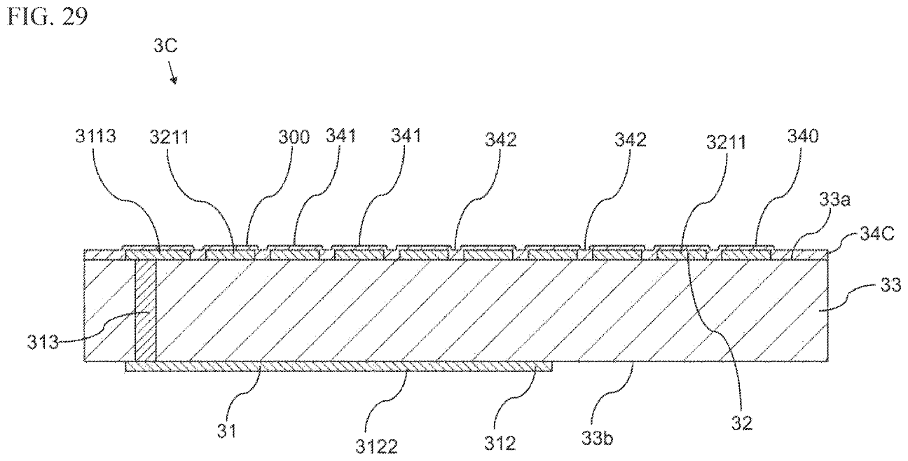

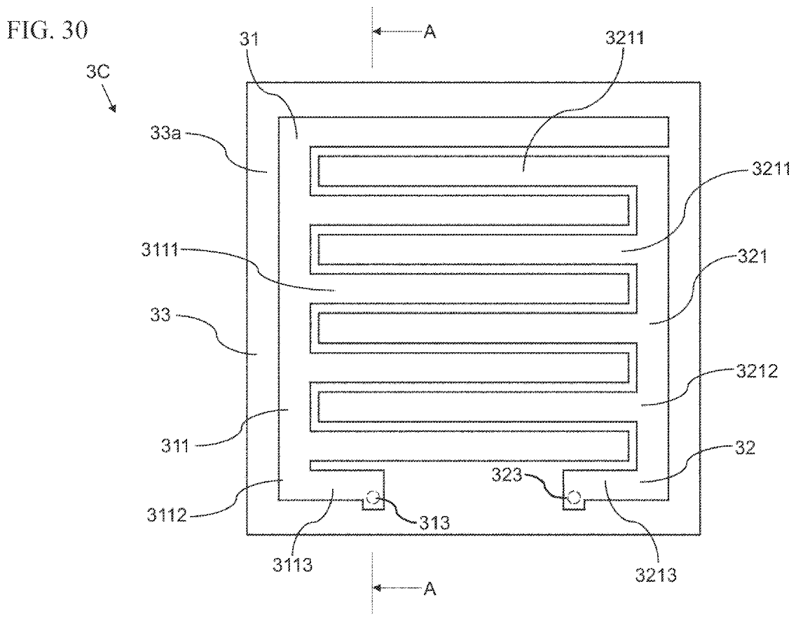

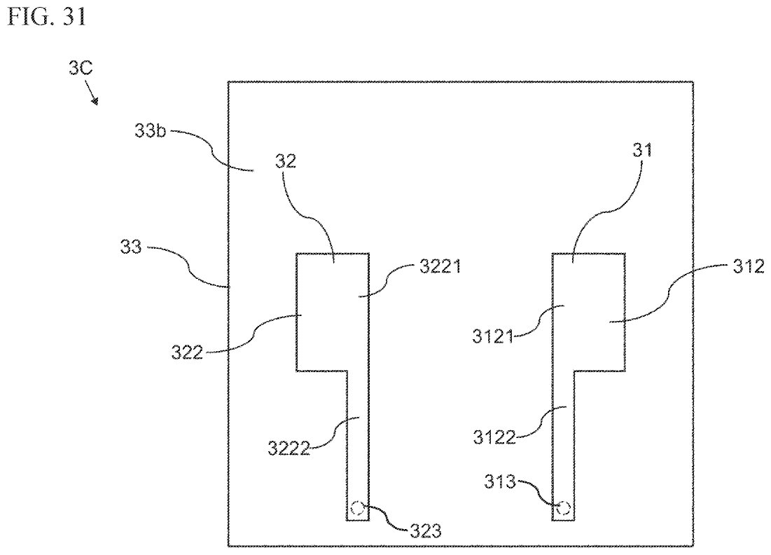

FIG. 28 is an explanatory diagram of a configuration example of the sensor unit 3C of the electrostatic capacity sensor 1C. FIG. 29 is a schematic cross-sectional view of the configuration example of the sensor unit 3C of the electrostatic capacity sensor 1C. FIG. 30 is a schematic plan view of the sensor unit 3C. FIG. 31 is a schematic bottom view of the sensor unit 3C. In particular, FIG. 29 is a cross-sectional view taken along line A-A of FIG. 30.

The sensor unit 3C in FIGS. 28 and 29 includes the first electrode 31, the second electrode 32, the sensor substrate 33, and a protective layer 34C. The sensor unit 3C is formed such that the first and second electrodes 31 and 32 form the capacitor 30 (see FIG. 2) together with a part of the measurement target by bringing the first and second electrodes 31 and 32 into contact with the measurement target.

As illustrated in FIGS. 30 and 31, the sensor substrate 33 has a rectangular plate shape. As illustrated in FIGS. 28 and 29, the sensor substrate 33 has the first surface 33a and the second surface 33b in a thickness direction of the sensor substrate 33. As illustrated in FIGS. 28 and 29, the first electrode 31, the second electrode 32, and the protective layer 34C are disposed on the sensor substrate 33. In FIG. 30, the illustration of the protective layer 34C is omitted.

As illustrated in FIGS. 30 and 31, the first electrode 31 includes the electrode portion 311, the terminal portion 312, and the connection portion 313.

The electrode portion 311 is used for contact with the measurement target. As illustrated in FIG. 30, the electrode portion 311 is disposed on the first surface 33a of the sensor substrate 33. The electrode portion 311 in FIG. 30 has a comb tooth structure. The electrode portion 311 includes the plurality of tooth portions 3111 disposed at a predetermined interval, the coupling portion 3112 that couples one ends of the plurality of tooth portions 3111 to each other, and a connection portion 3113 that is connected to the terminal portion 312. The connection portion 3113 extends from an end portion of the coupling portion 3112 to be arranged with the plurality of tooth portions 3111. As illustrated in FIG. 28, the electrode portion 311 includes, for example, a plurality of metal layers. The plurality of metal layers of the electrode portion 311 in FIG. 28 includes, for example, the Ni layer 311a, the Pd layer 311b that covers the Ni layer 311a, and the Au layer 311c that covers the Pd layer 311b. The plurality of metal layers of the electrode portion 311 can be formed by plating processing.

The terminal portion 312 is used for the connection to the electrostatic capacity detection circuit 4. As illustrated in FIG. 31, the terminal portion 312 is disposed on the second surface 33b of the sensor substrate 33. The terminal portion 312 in FIG. 31 has a rectangular pad portion 3121 and a connection portion 3122 that is connected to the electrode portion 311. The connection portion 3122 is in a belt shape extending from the pad portion 3121. As illustrated in FIG. 28, the terminal portion 312 includes, for example, a plurality of metal layers (metal films). The plurality of metal layers of the terminal portion 312 in FIG. 28 includes, for example, the Ni layer 312a, the Pd layer 312b that covers the Ni layer 312a, and the Au layer 312c that covers the Pd layer 312b. The plurality of metal layers of the terminal portion 312 can be formed by plating processing.

The connection portion 313 connects the electrode portion 311 and the terminal portion 312. More specifically, the connection portion 313 connects an end portion of the connection portion 3113 of the electrode portion 311 and an end portion of the connection portion 3122 of the terminal portion 312. As illustrated in FIG. 29, the connection portion 313 is a via that penetrates the sensor substrate 33. The connection portion 313 is made of, for example, Ag.

As illustrated in FIGS. 30 and 31, the second electrode 32 includes the electrode portion 321, the terminal portion 322, and the connection portion 323.

The electrode portion 321 is used for contact with the measurement target. As illustrated in FIG. 30, the electrode portion 321 is disposed on the first surface 33a of the sensor substrate 33. The electrode portion 321 in FIG. 30 has a comb tooth structure. The electrode portion 321 includes a plurality of tooth portions 3211 disposed at a predetermined interval, a coupling portion 3212 that couples one ends of the plurality of tooth portions 3211 to each other, and a connection portion 3213 that is connected to the terminal portion 322. The connection portion 3213 extends from an end portion of the coupling portion 3212 to be arranged with the plurality of tooth portions 3211. As illustrated in FIG. 28, the electrode portion 321 includes, for example, a plurality of metal layers. The plurality of metal layers of the electrode portion 321 in FIG. 28 includes, for example, the Ni layer 321a, the Pd layer 321b that covers the Ni layer 321a, and the Au layer 321c that covers the Pd layer 321b. The plurality of metal layers of the electrode portion 321 can be formed by plating processing.

The terminal portion 322 is used for the connection to the electrostatic capacity detection circuit 4. As illustrated in FIG. 31, the terminal portion 322 is disposed on the second surface 33b of the sensor substrate 33. The terminal portion 322 in FIG. 31 has a rectangular pad portion 3221 and a connection portion 3222 that is connected to the electrode portion 321. The connection portion 3222 is in a belt shape extending from the pad portion 3221. As illustrated in FIG. 28, the terminal portion 322 includes, for example, a plurality of metal layers (metal films). The plurality of metal layers of the terminal portion 322 in FIG. 28 includes, for example, the Ni layer 322a, the Pd layer 322b that covers the Ni layer 322a, and the Au layer 322c that covers the Pd layer 322b. The plurality of metal layers of the terminal portion 322 can be formed by plating processing.

The connection portion 323 connects the electrode portion 321 and the terminal portion 322. More specifically, the connection portion 323 connects an end portion of the connection portion 3213 of the electrode portion 321 and an end portion of the connection portion 3222 of the terminal portion 322. The connection portion 323 is a via that penetrates the sensor substrate 33. The connection portion 323 is made of, for example, Ag.

The protective layer 34C is used to protect the first electrode 31 and the second electrode 32. In particular, the protective layer 34C is used to protect the electrode portion 311 of the first electrode 31 and the electrode portion 321 of the second electrode 32. As illustrated in FIGS. 28 and 29, the protective layer 34C is disposed on the first surface 33a of the sensor substrate 33. The protective layer 34C covers the electrode portion 311 of the first electrode 31 and the electrode portion 321 of the second electrode 32. The protective layer 34C has, for example, insulating properties. The protective layer 34C is made of, for example, a material having insulating properties, such as polyimide.

A surface 340 of the protective layer 34C in FIG. 28 has an uneven shape. In FIG. 28, the surface 340 of the protective layer 34C includes a protruding region 341 and a recessed region 342. The protruding region 341 includes a region that covers the electrode portion 311 of the first electrode 31 or the electrode portion 321 of the second electrode 32, and the recessed region 342 does not include a region that covers the electrode portion 311 of the first electrode 31 or the electrode portion 321 of the second electrode 32. In other words, the surface 340 of the protective layer 34C in FIG. 28 reflects the uneven shape generated by forming the electrode portions 311 and 321 on the first surface 33a of the sensor substrate 33. As described above, in a case where the protective layer 34C reflects unevenness of a base, generally, a thickness of the protective layer 34C is approximately constant. In other words, a thickness TH1 of the protective layer 34C in the protruding region 341 and a thickness TH2 of the protective layer 34C in the recessed region 342 are equal and substantially equal. The thickness TH1 of the protective layer 34C in the protruding region 341 is a distance between the protruding region 341 and the electrode portion 311 of the first electrode 31 or the electrode portion 321 of the second electrode 32. The thickness TH2 of the protective layer 34C in the recessed region 342 is a distance between the recessed region 342 and the first surface 33a. The protective layer 34C in FIG. 28 can be formed by, for example, a spin coating method. Note that, in the protective layer 34C, the thickness TH1 and the thickness TH2 are not necessarily substantially equal, and for example, as illustrated in FIG. 29, the thicknesses of the protective layer 34C in the protruding region 341 and the recessed region 342 may be different. Note that, in FIG. 29, the protective layer 34C is thicker in the recessed region 342 than in the protruding region 341.

In the present embodiment, the surface 340 of the protective layer 34C defines the surface 300 exposed from the head portion 21C in the sensor unit 3C. Then, as described above, the surface 340 of the protective layer 34C has an uneven shape, and thus, the surface 300 of the sensor unit 3C has an uneven shape.

As compared to a case where the surface 300 of the sensor unit 3C is flat, the surface 300 of the sensor unit 3C has an uneven shape, and thus, a specific surface area of the sensor unit 3C is large. When the specific surface area of the sensor unit 3C is large, the electrostatic capacity of the capacitor 30 constituted by the sensor unit 3C and a part of the measurement target can be large. Thus, it is possible to improve the detection accuracy of the electrostatic capacity. In particular, in the sensor unit 3C in FIGS. 28 and 29, a distance between the measurement target and the electrode portion 311 or the electrode portion 321 is likely to be reduced in the recessed region 342, and thus, the electrostatic capacity can be partially large.

As compared to a case where the surface 300 of the sensor unit 3C is flat, the surface 300 of the sensor unit 3C has an uneven shape, and thus, a friction coefficient (mainly, static friction coefficient) of the surface 300 of the sensor unit 3C with respect to the measurement target is large. As a result, a possibility that a positional relationship between the sensor unit 3C and the measurement target fluctuates at the time of measurement can be reduced. That is, a grip force of the sensor unit 3C is improved, and the sensor unit 3C is fixed by the measurement target. As a result, the surface 300 of the sensor unit 3C is easily pressed against the measurement target, and the pressure applied to the surface 300 of the sensor unit 3C by the measurement target is likely to be large. The pressure applied to the surface 300 of the sensor unit 3C is large depending on the measurement target. As a result, since the close contact of the sensor unit 3C to the measurement target is improved, the measurement is stabilized, and the detection accuracy of the electrostatic capacity can be improved.

### 1.4.2 Effects and the Like

In the measuring instrument 10C described above, the sensor unit 3C has the surface 300 exposed from the head portion 21C. The surface 300 of the sensor unit 3C has an uneven shape. As compared to a case where the surface 300 of the sensor unit 3C is flat, this configuration can increase the specific surface area of the sensor unit 3C and the friction coefficient (mainly, static friction coefficient) of the surface 300 of the sensor unit 3C with respect to the measurement target and can improve the detection accuracy of the electrostatic capacity.

The measuring instrument 10C described above has the contact region 100 that comes into contact with the measurement target. The contact region 100 includes the surface 300 of the sensor unit 3C and the frame-shaped region 200 surrounding the surface 300 of the sensor unit 3C in the head portion 21C. The contact region 100 has an uneven shape. As compared to a case where the contact region 100 is flat, this configuration can increase the friction coefficient (mainly, static friction coefficient) of the contact region 100, and can improve the detection accuracy of the electrostatic capacity.

In the measuring instrument 10C, the surface 300 of the sensor unit 3C has an uneven shape. As compared to a case where the surface 300 of the sensor unit 3C is flat, this configuration can increase the specific surface area of the sensor unit 3C and the friction coefficient (mainly, static friction coefficient) of the surface 300 of the sensor unit 3C with respect to the measurement target and can improve the detection accuracy of the electrostatic capacity.

### 1.5 Fifth Embodiment

### 1.5.1 Configuration

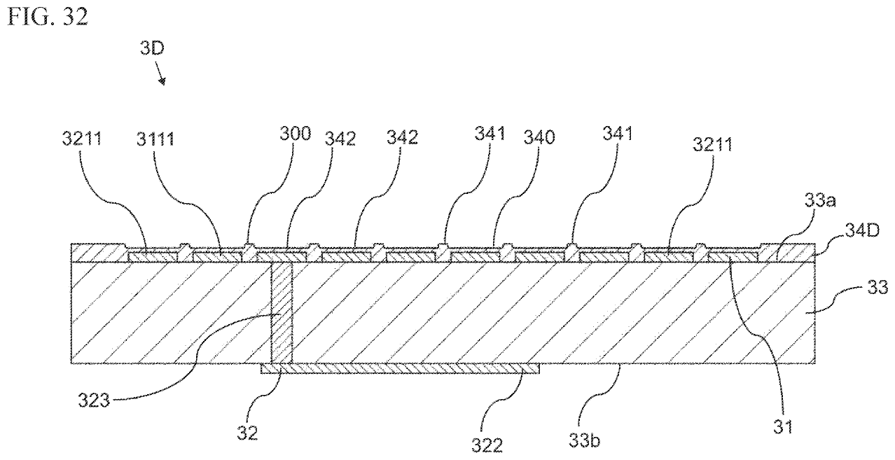

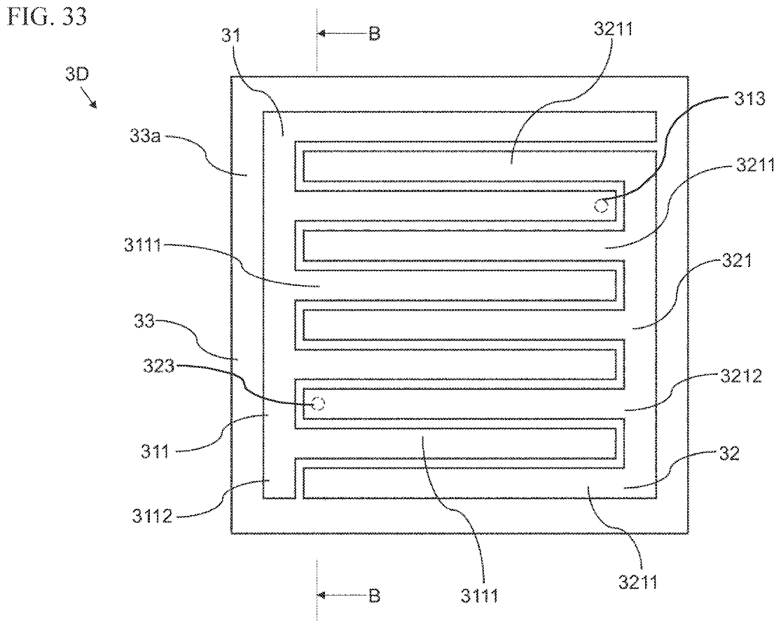

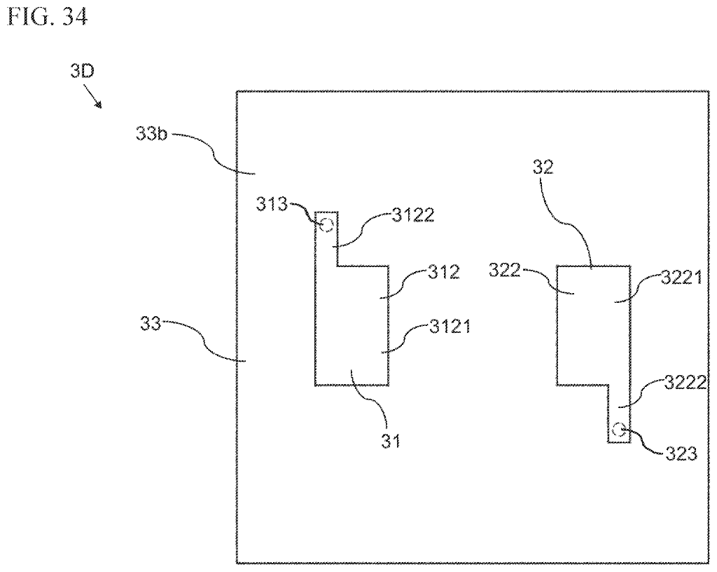

FIG. 32 is a schematic cross-sectional view of a configuration example of a sensor unit 3D of an electrostatic capacity sensor. FIG. 33 is a schematic plan view of the sensor unit 3D. FIG. 34 is a schematic bottom view of the sensor unit 3D. In particular, FIG. 32 is a cross-sectional view taken along line B-B of FIG. 33.

As with the sensor unit C, the sensor unit 3D is positioned in the head portion 21C of the handheld housing 2C. The surface 300 of the sensor unit 3D is exposed to an outside from the head portion 21C of the handheld housing 2C. The surface 300 of the sensor unit 3D and the frame-shaped region 200 surrounding the surface 300 of the sensor unit 3D in the head portion 21C constitute a contact region 100 that comes into contact with the measurement target.

The sensor unit 3D in FIG. 32 includes the first electrode 31, the second electrode 32, the sensor substrate 33, and a protective layer 34D. The sensor unit 3D is formed such that the first and second electrodes 31 and 32 form the capacitor 30 (see FIG. 2) together with a part of the measurement target by bringing the first and second electrodes 31 and 32 into contact with the measurement target.

As illustrated in FIGS. 33 and 34, the sensor substrate 33 has a rectangular plate shape. As illustrated in FIG. 32, the sensor substrate 33 has the first surface 33a and the second surface 33b in the thickness direction of the sensor substrate 33. As illustrated in FIG. 32, the first electrode 31, the second electrode 32, and the protective layer 34D are disposed on the sensor substrate 33. In FIG. 33, the illustration of the protective layer 34D is omitted.

As illustrated in FIGS. 33 and 34, the first electrode 31 includes the electrode portion 311, the terminal portion 312, and the connection portion 313.

The electrode portion 311 is used for contact with the measurement target. As illustrated in FIG. 33, the electrode portion 311 is disposed on the first surface 33a of the sensor substrate 33. The electrode portion 311 in FIG. 33 has a comb tooth structure. The electrode portion 311 includes a plurality of tooth portions 3111 arranged at a predetermined interval, and a coupling portion 3112 that couples one ends of the plurality of tooth portions 3111 to each other. The electrode portion 311 includes, for example, a plurality of metal layers. The plurality of metal layers of the electrode portion 311 includes, for example, a Ni layer, a Pd layer that covers the Ni layer, and an Au layer that covers the Pd layer. The plurality of metal layers of the electrode portion 311 can be formed by plating processing.

The terminal portion 312 is used for the connection to the electrostatic capacity detection circuit 4. As illustrated in FIG. 34, the terminal portion 312 is disposed on the second surface 33b of the sensor substrate 33. The terminal portion 312 in FIG. 34 has the rectangular pad portion 3121 and the connection portion 3122 that is connected to the electrode portion 311. The connection portion 3122 is in a belt shape extending from the pad portion 3121. The terminal portion 312 includes, for example, a plurality of metal layers (metal films). The plurality of metal layers of the terminal portion 312 includes, for example, a Ni layer, a Pd layer that covers the Ni layer, and an Au layer that covers the Pd layer. The plurality of metal layers of the terminal portion 312 can be formed by plating processing.

The connection portion 313 connects the electrode portion 311 and the terminal portion 312. More specifically, the connection portion 313 connects one end portions of the plurality of tooth portions 3111 of the electrode portion 311 to the end portion of the connection portion 3122 of the terminal portion 312. The connection portion 313 is a via that penetrates the sensor substrate 33. The connection portion 313 is made of, for example, Ag.

As illustrated in FIGS. 33 and 34, the second electrode 32 has the electrode portion 321, the terminal portion 322, and the connection portion 323.

The electrode portion 321 is used for contact with the measurement target. As illustrated in FIG. 33, the electrode portion 321 is disposed on the first surface 33a of the sensor substrate 33. The electrode portion 321 in FIG. 33 has a comb tooth structure. The electrode portion 321 includes a plurality of tooth portions 3211 arranged at a predetermined interval, and a coupling portion 3212 that couples one ends of the plurality of tooth portions 3211 to each other. The electrode portion 321 includes, for example, a plurality of metal layers. The plurality of metal layers of the electrode portion 321 includes, for example, a Ni layer, a Pd layer that covers the Ni layer, and an Au layer that covers the Pd layer. The plurality of metal layers of the electrode portion 321 can be formed by plating processing.

The terminal portion 322 is used for the connection to the electrostatic capacity detection circuit 4. As illustrated in FIG. 34, the terminal portion 322 is disposed on the second surface 33b of the sensor substrate 33. The terminal portion 322 in FIG. 34 has the rectangular pad portion 3221 and the connection portion 3222 that is connected to the electrode portion 321. The connection portion 3222 is in a belt shape extending from the pad portion 3221. The terminal portion 322 includes, for example, a plurality of metal layers (metal films). The plurality of metal layers of the terminal portion 322 includes, for example, a Ni layer, a Pd layer that covers the Ni layer, and an Au layer that covers the Pd layer. The plurality of metal layers of the terminal portion 322 can be formed by plating processing.

The connection portion 323 connects the electrode portion 321 and the terminal portion 322. More specifically, the connection portion 323 connects one end portions of the plurality of tooth portions 3211 of the electrode portion 321 to the end portion of the connection portion 3222 of the terminal portion 322. As illustrated in FIG. 32, the connection portion 323 is a via that penetrates the sensor substrate 33. The connection portion 323 is made of, for example, Ag.

The protective layer 34D is used to protect the first electrode 31 and the second electrode 32. In particular, the protective layer 34D is used to protect the electrode portion 311 of the first electrode 31 and the electrode portion 321 of the second electrode 32. As illustrated in FIG. 32, the protective layer 34D is disposed on the first surface 33a of the sensor substrate 33. The protective layer 34D covers the electrode portion 311 of the first electrode 31 and the electrode portion 321 of the second electrode 32. The protective layer 34D has, for example, insulating properties. The protective layer 34D is made of, for example, a material having insulating properties, such as polyimide.

The surface 340 of the protective layer 34D in FIG. 32 has an uneven shape. In FIG. 32, the surface 340 of the protective layer 34D includes the protruding region 341 and the recessed region 342. A distance from the first surface 33a of the sensor substrate 33 in the recessed region 342 is shorter than a distance from the first surface 33a of the sensor substrate 33 in the protruding region 341. The protruding region 341 does not include a region that covers the electrode portion 311 of the first electrode 31 or the electrode portion 321 of the second electrode 32, and the recessed region 342 includes a region that covers the electrode portion 311 of the first electrode 31 or the electrode portion 321 of the second electrode 32.

In the present embodiment, the surface 340 of the protective layer 34D defines the surface 300 exposed from the head portion 21C in the sensor unit 3D. Then, as described above, the surface 340 of the protective layer 34D has an uneven shape, and thus, the surface 300 of the sensor unit 3D has an uneven shape.

As compared to a case where the surface 300 of the sensor unit 3D is flat, the surface 300 of the sensor unit 3D has an uneven shape, and thus, the specific surface area of the sensor unit 3D is large. When the specific surface area of the sensor unit 3D is large, the electrostatic capacity of the capacitor 30 constituted by the sensor unit 3D together with a part of the measurement target can be large. Thus, it is possible to improve the detection accuracy of the electrostatic capacity. In particular, in the sensor unit 3D in FIG. 32, the distance between the measurement target and the electrode portion 311 or the electrode portion 321 is likely to be reduced in the recessed region 342, and thus, the electrostatic capacity can be partially large.

As compared to a case where the surface 300 of the sensor unit 3D is flat, the surface 300 of the sensor unit 3D has an uneven shape, and thus, the friction coefficient (mainly, static friction coefficient) of the surface 300 of the sensor unit 3D with respect to the measurement target is large. As a result, a possibility that a positional relationship between the sensor unit 3D and the measurement target fluctuates at the time of measurement can be reduced. That is, a grip force of the sensor unit 3D is improved, and the sensor unit 3D is fixed by the measurement target. As a result, the surface 300 of the sensor unit 3D is easily pressed against the measurement target, and the pressure applied to the surface 300 of the sensor unit 3D by the measurement target is likely to be large. The pressure applied to the surface 300 of the sensor unit 3D is large depending on the measurement target. As a result, since the close contact of the sensor unit 3D to the measurement target is improved, the measurement is stabilized, and the detection accuracy of the electrostatic capacity can be improved.

### 1.5.2 Effects and the Like

In the measuring instrument 10D described above, the sensor unit 3D has the surface 300 exposed from the head portion 21C. The surface 300 of the sensor unit 3D has an uneven shape. As compared to a case where the surface 300 of the sensor unit 3D is flat, this configuration can increase the specific surface area of the sensor unit 3D and the friction coefficient (mainly, static friction coefficient) of the surface 300 of the sensor unit 3D with respect to the measurement target and can improve the detection accuracy of the electrostatic capacity.

The measuring instrument 10D described above has the contact region 100 that comes into contact with the measurement target. The contact region 100 includes the surface 300 of the sensor unit 3D and the frame-shaped region 200 surrounding the surface 300 of the sensor unit 3D in the head portion 21C. The contact region 100 has an uneven shape. As compared to a case where the contact region 100 is flat, this configuration can increase the friction coefficient (mainly, static friction coefficient) of the contact region 100, and can improve the detection accuracy of the electrostatic capacity.

In the measuring instrument 10D, the surface 300 of the sensor unit 3D has an uneven shape. As compared to a case where the surface 300 of the sensor unit 3D is flat, this configuration can increase the specific surface area of the sensor unit 3D and the friction coefficient (mainly, static friction coefficient) of the surface 300 of the sensor unit 3D with respect to the measurement target and can improve the detection accuracy of the electrostatic capacity.

### 1.6 Six Embodiment

### 1.6.1 Configuration

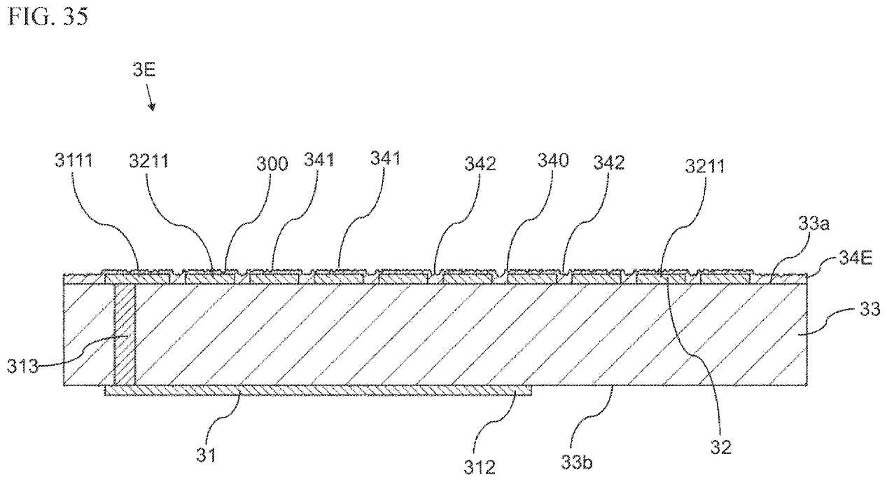

FIG. 35 is a schematic cross-sectional view of a configuration example of a sensor unit 3E of an electrostatic capacity sensor of a measuring instrument according to a sixth embodiment.

As with the sensor unit C, the sensor unit 3E is positioned at the head portion 21C of the handheld housing 2C. The surface 300 of the sensor unit 3E is exposed to an outside from the head portion 21C of the handheld housing 2C. The surface 300 of the sensor unit 3E and the frame-shaped region 200 surrounding the surface 300 of the sensor unit 3E in the head portion 21C constitute a contact region 100 that comes into contact with the measurement target.

The sensor unit 3E in FIG. 35 includes the first electrode 31, the second electrode 32, the sensor substrate 33, and a protective layer 34E. The first electrode 31, the second electrode 32, and the sensor substrate 33 of the sensor unit 3E are similar to the first electrode 31, the second electrode 32, and the sensor substrate 33 of the sensor unit 3C.

The surface 340 of the protective layer 34E in FIG. 35 has an uneven shape, as with the surface 340 of the protective layer 34C in FIGS. 28 and 29. In FIG. 35, the surface 340 of the protective layer 34E includes the protruding region 341 and the recessed region 342. A distance from the first surface 33a of the sensor substrate 33 in the recessed region 342 is shorter than a distance from the first surface 33a of the sensor substrate 33 in the protruding region 341. The protruding region 341 includes a region that covers the electrode portion 311 of the first electrode 31 or the electrode portion 321 of the second electrode 32, and the recessed region 342 does not include a region that covers the electrode portion 311 of the first electrode 31 or the electrode portion 321 of the second electrode 32.

On the surface 340 of the protective layer 34E in FIG. 35, a surface having the uneven shape is a rough surface having irregular unevenness. More specifically, each of the protruding region 341 and the recessed region 342 of the surface 340 of the protective layer 34E has a rough surface with irregular unevenness. A known technique such as an etching technique can be used to roughen the uneven shape of the surface 340 of the protective layer 34E.

In the present embodiment, the surface 340 of the protective layer 34E, that is, the surface having the uneven shape on the surface 300 of the sensor unit 3E has irregular unevenness, and thus, the specific surface area of the sensor unit 3E is further large, and the friction coefficient (mainly, static friction coefficient) of the surface 300 of the sensor unit 3E with respect to the measurement target is further large. As a result, it is possible to further improve the detection accuracy of the electrostatic capacity.

### 1.6.2 Effects and the Like

As described above, the sensor unit 3E has the surface 300 exposed from the head portion 21C. The surface 300 of the sensor unit 3E has an uneven shape. As compared to a case where the surface 300 of the sensor unit 3E is flat, this configuration can increase the specific surface area of the sensor unit 3E and the friction coefficient (mainly, static friction coefficient) of the surface 300 of the sensor unit 3E with respect to the measurement target and can improve the detection accuracy of the electrostatic capacity.

In the sensor unit 3E, the surface having the uneven shape on the surface 300 of the sensor unit 3E is a rough surface having irregular unevenness. This configuration can further increase the specific surface area of the sensor unit 3E and the friction coefficient (mainly, static friction coefficient) of the surface 300 of the sensor unit 3E with respect to the measurement target, and can further improve the detection accuracy of the electrostatic capacity.

### 1.7 Seventh Embodiment

### 1.7.1 Configuration

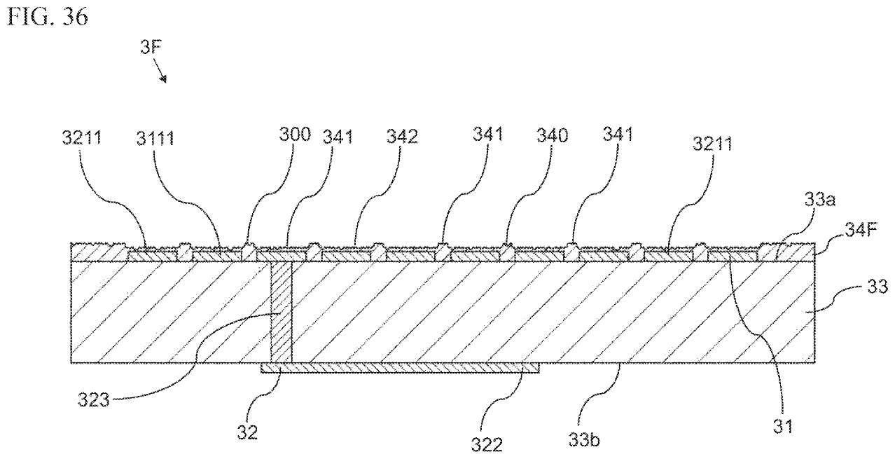

FIG. 36 is a schematic cross-sectional view of a configuration example of a sensor unit 3F of an electrostatic capacity sensor of a measuring instrument according to a seventh embodiment.

As with the sensor unit C, the sensor unit 3F is positioned at the head portion 21C of the handheld housing 2C. The surface 300 of the sensor unit 3F is exposed to an outside from the head portion 21C of the handheld housing 2C. The surface 300 of the sensor unit 3F and the frame-shaped region 200 surrounding the surface 300 of the sensor unit 3F in the head portion 21C constitute a contact region 100 that comes into contact with the measurement target.

The sensor unit 3F in FIG. 36 includes the first electrode 31, the second electrode 32, the sensor substrate 33, and a protective layer 34F. The first electrode 31, the second electrode 32, and the sensor substrate 33 of the sensor unit 3F are similar to the first electrode 31, the second electrode 32, and the sensor substrate 33 of the sensor unit 3D.

The surface 340 of the protective layer 34F in FIG. 36 has an uneven shape, as with the surface 340 of the protective layer 34D in FIG. 32. In FIG. 36, the surface 340 of the protective layer 34F includes the protruding region 341 and the recessed region 342. A distance from the first surface 33a of the sensor substrate 33 in the recessed region 342 is shorter than a distance from the first surface 33a of the sensor substrate 33 in the protruding region 341. The protruding region 341 does not include a region that covers the electrode portion 311 of the first electrode 31 or the electrode portion 321 of the second electrode 32, and the recessed region 342 includes a region that covers the electrode portion 311 of the first electrode 31 or the electrode portion 321 of the second electrode 32.

On the surface 340 of the protective layer 34F in FIG. 36, the surface having the uneven shape is a rough surface having irregular unevenness. More specifically, each of the protruding region 341 and the recessed region 342 of the surface 340 of the protective layer 34F has a rough surface with irregular unevenness. A known technique such as an etching technique can be used to roughen the uneven shape of the surface 340 of the protective layer 34F.

In the present embodiment, the surface 340 of the protective layer 34F, that is, the surface having the uneven shape on the surface 300 of the sensor unit 3F has irregular unevenness, and thus, the specific surface area of the sensor unit 3F is further large, and thus, the friction coefficient (mainly, static friction coefficient) of the surface 300 of the sensor unit 3F with respect to the measurement target is further large. As a result, it is possible to further improve the detection accuracy of the electrostatic capacity.

### 1.7.2 Effects and the Like

As described above, the sensor unit 3F has the surface 300 exposed from the head portion 21C. The surface 300 of the sensor unit 3F has an uneven shape. As compared to a case where the surface 300 of the sensor unit 3F is flat, this configuration can increase the specific surface area of the sensor unit 3F and the friction coefficient (mainly, static friction coefficient) of the surface 300 of the sensor unit 3F with respect to the measurement target and can improve the detection accuracy of the electrostatic capacity.

In the sensor unit 3F, the surface having the uneven shape on the surface 300 of the sensor unit 3F is a rough surface having irregular unevenness. This configuration can further increase the specific surface area of the sensor unit 3F and the friction coefficient (mainly, static friction coefficient) of the surface 300 of the sensor unit 3F with respect to the measurement target and can further improve the detection accuracy of the electrostatic capacity.

### 1.8 Eighth Embodiment

### 1.8.1 Configuration

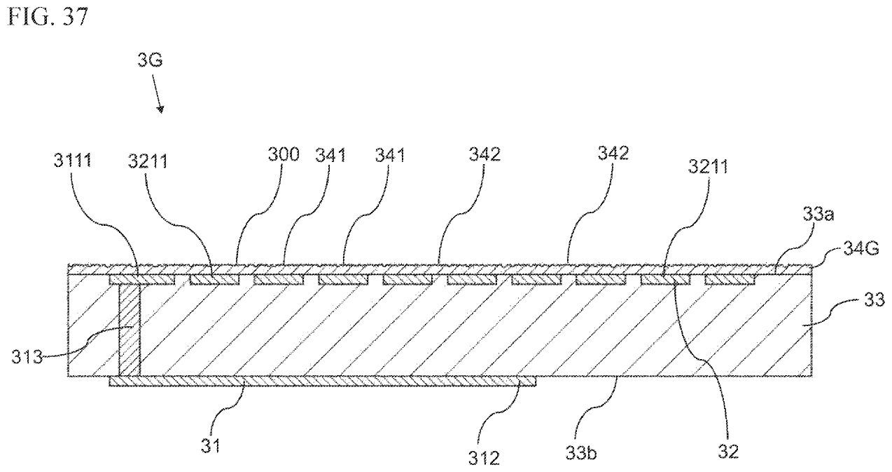

FIG. 37 is a schematic cross-sectional view of a configuration example of a sensor unit 3G of an electrostatic capacity sensor of a measuring instrument according to an eighth embodiment.

As with the sensor unit C, the sensor unit 3G is positioned at the head portion 21C of the handheld housing 2C. The surface 300 of the sensor unit 3G is exposed to the outside from the head portion 21C of the handheld housing 2C. The surface 300 of the sensor unit 3G and the frame-shaped region 200 surrounding the surface 300 of the sensor unit 3G in the head portion 21C constitute a contact region 100 that comes into contact with the measurement target. The sensor unit 3G in FIG. 37 includes the first

electrode 31, the second electrode 32, the sensor substrate 33, and a protective layer 34G. The first electrode 31, the second electrode 32, and the sensor substrate 33 of the sensor unit 3G are similar to the first electrode 31, the second electrode 32, and the sensor substrate 33 of the sensor unit 3C. However, in the sensor unit 3G in FIG. 37, the electrode portion 311 of the first electrode 31 and the electrode portion 321 of the second electrode 32 are positioned on the first surface 33a of the sensor substrate 33, but the surface of the electrode portion 311 of the first electrode 31 and the surface of the electrode portion 321 of the second electrode 32 are positioned on the same plane as the first surface 33a of the sensor substrate 33.

The surface 340 of the protective layer 34G in FIG. 37 does not have an uneven shape like the surface 340 of the protective layer 34C in FIGS. 28 and 29, but includes a rough surface having irregular unevenness. A known technique such as an etching technique can be used to roughen the surface 340 of the protective layer 34G. Note that, the surface 340 of the protective layer 34C in FIGS. 28 and 29 is a rough surface having irregular unevenness as a whole, but the entire surface 340 does not necessarily have to be a rough surface having irregular unevenness.

In the present embodiment, the surface 340 of the protective layer 34G, that is, the surface 300 of the sensor unit 3G is a rough surface having irregular unevenness, and thus, the specific surface area of the sensor unit 3G is large, and the friction coefficient (mainly, static friction coefficient) of the surface 300 of the sensor unit 3G with respect to the measurement target is large. As a result, it is possible to improve the detection accuracy of the electrostatic capacity.

### 1.8.2 Effects and the Like

As described above, the sensor unit 3G has the surface 300 exposed from the head portion 21C. The surface 300 of the sensor unit 3G includes a rough surface. As compared to a case where the surface 300 of the sensor unit 3G is flat, this configuration can increase the specific surface area of the sensor unit 3G and the friction coefficient (mainly, static friction coefficient) of the surface 300 of the sensor unit 3G with respect to the measurement target and can improve the detection accuracy of the electrostatic capacity.

In the sensor unit 3G, the surface having the uneven shape on the surface 300 of the sensor unit 3G is a rough surface. This configuration can increase the specific surface area of the sensor unit 3G and the friction coefficient (mainly, static friction coefficient) of the surface 300 of the sensor unit 3G with respect to the measurement target and can improve the detection accuracy of the electrostatic capacity.

### 1.9 Ninth Embodiment

### 1.9.1 Configuration

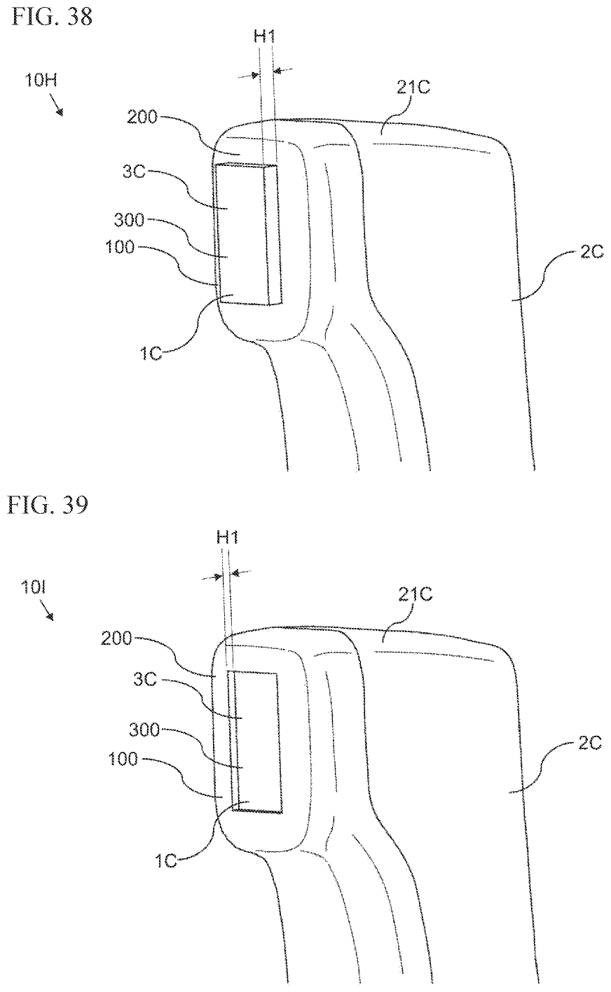

FIG. 38 is a schematic perspective view of a configuration example of a head portion 21C of a measuring instrument 10H according to a ninth embodiment. As with the measuring instrument 10C, the measuring instrument 10H is an electrostatic capacity type moisture measuring instrument. The measuring instrument 10H includes the electrostatic capacity sensor 1C and the handheld housing 2C.

The handheld housing 2C accommodates the electrostatic capacity sensor 1C. The handheld housing 2C includes the head portion 21C. Although not illustrated in FIG. 38, the handheld housing 2C includes the grip portion 22 and the probe portion 23, as with the handheld housing 2 in FIG. 1.

The electrostatic capacity sensor 1C calculates the moisture content of the measurement target based on the electrostatic capacity. The electrostatic capacity sensor 1C includes a sensor unit 3C. As with the electrostatic capacity sensor 1 of FIG. 2, the electrostatic capacity sensor 1C includes the electrostatic capacity detection circuit 4 and the processing circuit 5.

In the present embodiment, at least the sensor unit 3C is positioned in the head portion 21C of the handheld housing 2C. A surface 300 of the sensor unit 3C is exposed to an outside from the head portion 21C of the handheld housing 2C. The surface 300 of the sensor unit 3C and the frame-shaped region 200 surrounding the surface 300 of the sensor unit 3C in the head portion 21C constitute a contact region 100 that comes into contact with the measurement target.

The surface 300 of the sensor unit 3C in FIG. 38 protrudes with respect to the frame-shaped region 200 of the head portion 21C. Accordingly, in a case where the contact region 100 of the measuring instrument 10H is brought into contact with the measurement target, the surface 300 of the sensor unit 3C is sufficiently likely to come into contact with the measurement target, the variation in the measurement of the measuring instrument 10H is suppressed, and it is possible to improve the detection accuracy of the electrostatic capacity. In particular, the sensor unit 3C is more likely to come into contact with the measurement target in a case where the surface 300 of the sensor unit 3C protrudes with respect to the frame-shaped region 200 of the head portion 21C than in a case where the surface 300 of the sensor unit 3C is recessed with respect to the frame-shaped region 200 of the head portion 21C. Since the surface 300 of the sensor unit 3C protrudes with respect to the frame-shaped region 200 of the head portion 21C, even though the sensor unit 3C is charged in a state where static electricity is likely to be generated, such as a low humidity environment in winter, the static electricity charged in the sensor unit 3C can be effectively discharged. Thus, the variation in the measurement result due to the charging of the sensor unit 3C is suppressed, and thus, it is possible to improve the detection accuracy of the electrostatic capacity. In this case, when an area of the surface 300 of the sensor unit 3C is 1 mm² or more, the static electricity can be more effectively discharged.

In the present embodiment, the entire surface 300 of the sensor unit 3C protrudes from the frame-shaped region 200 of the head portion 21C. Accordingly, the sensor unit 3C comes into contact with the measurement target on the entire surface while obtaining the grip force. As a result, a signal (change in electrostatic capacity) detected by the sensor unit 3C is large. As a result, it is possible to improve the detection accuracy of the electrostatic capacity. Since the entire surface 300 of the sensor unit 3C protrudes from the frame-shaped region 200 of the head portion 21C, the static electricity charged on the sensor unit 3C is more effectively discharged. As a result, the variation in the measurement result due to the charging of the sensor unit 3C is suppressed, and thus, it is possible to improve the detection accuracy of the electrostatic capacity.

In the measuring instrument 10H in FIG. 38, a sensor height H1 is 5 μm or more and 1 mm or less. Since the sensor height H1 is 5 μm or more, the variation in the measurement of the measuring instrument 10H can be suppressed as compared with a case where the sensor height H1 is less than 5 μm. Since the sensor height H1 is 1 mm or less, a possibility that an excess pressure is applied to the measurement target when the sensor unit 3C comes into contact with the measurement target can be reduced, as compared to a case where the sensor height H1 is larger than 1 mm. In a case where the excess pressure is applied to the measurement target when the sensor unit 3C comes into contact with the measurement target, there is a possibility that pain is felt when the measurement target is a person.

The sensor height H1 in FIG. 38 is defined as a distance between the surface 300 of the sensor unit 3C and a predetermined plane including the frame-shaped region 200 of the head portion 21C. In the sensor unit 3C, as illustrated in FIGS. 28 and 29, the surface 300 of the sensor unit 3C has an uneven shape. Here, the thickness of the protective layer 34C is very thin. Thus, a distance between the first surface 33a of the sensor substrate 33 of the sensor unit 3C and the predetermined plane can be used as the distance between the surface 300 of the sensor unit 3C and the predetermined plane, that is, the sensor height H1. As a result, the sensor height H1 can be set regardless of the shape of the surface 300 of the sensor unit 3C.

### 1.9.2 Effects and the Like

In the measuring instrument 10H described above, the sensor unit 3C has the surface 300 exposed from the head portion 21C. The head portion 21C has the frame-shaped region 200 surrounding the surface 300 of the sensor unit 3C. At least a part of the surface 300 of the sensor unit 3C protrudes with respect to the frame-shaped region 200 of the head portion 21C. As compared to a case where at least a part of the surface 300 of the sensor unit 3C neither protrudes nor is recessed with respect to the frame-shaped region 200 of the head portion 21C, since the close contact of the sensor unit 3C to the measurement target is improved, the measurement is stabilized, and this configuration can improve the detection accuracy of the electrostatic capacity. Since the static electricity charged in the sensor unit 3C is effectively discharged, the variation in the measurement result due to the charging of the sensor unit 3C is suppressed, and thus, this configuration can improve the detection accuracy of the electrostatic capacity.

In the measuring instrument 10H, the entire surface 300 of the sensor unit 3C protrudes from the frame-shaped region 200 of the head portion 21C. Since the close contact of the sensor unit 3C to the measurement target is further improved, the measurement is stabilized, and thus, this configuration can further improve the detection accuracy of the electrostatic capacity. Since the static electricity charged in the sensor unit 3C is more effectively discharged, the variation in the measurement result due to the charging of the sensor unit 3C is suppressed, and thus, this configuration can improve the detection accuracy of the electrostatic capacity.

In the measuring instrument 10H, a distance (sensor height H1) between the surface 300 of the sensor unit 3C and the predetermined plane including the frame-shaped region 200 of the head portion 21C is 5 μm or more and 1 mm or less. This configuration can improve the detection accuracy of the electrostatic capacity while reducing a possibility that an excess pressure is applied to the measurement target when the sensor unit 3C comes into contact with the measurement target.

### 1.10 Tenth Embodiment

### 1.10.1 Configuration

FIG. 39 is a schematic perspective view of a configuration example of a head portion 21C of a measuring instrument 10I according to a tenth embodiment. As with the measuring instrument 10C, the measuring instrument 10I is an electrostatic capacity type moisture measuring instrument. The measuring instrument 10I includes the electrostatic capacity sensor 1C and the handheld housing 2C.

The handheld housing 2C accommodates the electrostatic capacity sensor 1C. The handheld housing 2C includes the head portion 21C. Although not illustrated in FIG. 39, the handheld housing 2C includes the grip portion 22 and the probe portion 23, as with the handheld housing 2 in FIG. 1.

The electrostatic capacity sensor 1C calculates the moisture content of the measurement target based on the electrostatic capacity. The electrostatic capacity sensor 1C includes a sensor unit 3C. As with the electrostatic capacity sensor 1 of FIG. 2, the electrostatic capacity sensor 1C includes the electrostatic capacity detection circuit 4 and the processing circuit 5.

In the present embodiment, at least the sensor unit 3C is positioned in the head portion 21C of the handheld housing 2C. A surface 300 of the sensor unit 3C is exposed to an outside from the head portion 21C of the handheld housing 2C. The surface 300 of the sensor unit 3C and the frame-shaped region 200 surrounding the surface 300 of the sensor unit 3C in the head portion 21C constitute a contact region 100 that comes into contact with the measurement target.

The surface 300 of the sensor unit 3C in FIG. 39 is recessed with respect to the frame-shaped region 200 of the head portion 21C. As a result, in a case where the contact region 100 of the measuring instrument 10I is brought into contact with the measurement target, a part of the measurement target is deformed and enters a space surrounded by the frame-shaped region 200 of the head portion 21C. As a result, the surface 300 of the sensor unit 3C is sufficiently likely to come into contact with the measurement target, the variation in the measurement of the measuring instrument 10I is suppressed, and it is possible to improve the detection accuracy of the electrostatic capacity. In particular, the grip force is more improved in a case where the surface 300 of the sensor unit 3C is recessed with respect to the frame-shaped region 200 of the head portion 21C than in a case where the surface 300 of the sensor unit 3C protrudes with respect to the frame-shaped region 200 of the head portion 21C, and the sensor unit 3C is easily fixed to the measurement target.

In the present embodiment, the entire surface 300 of the sensor unit 3C is recessed from the frame-shaped region 200 of the head portion 21C. Accordingly, the sensor unit 3C is more likely to come into contact with the measurement target over the entire surface while obtaining the grip force. As a result, a signal (change in electrostatic capacity) detected by the sensor unit 3C is large. As a result, it is possible to improve the detection accuracy of the electrostatic capacity.

In the measuring instrument 10I in FIG. 39, the sensor height I1 is 5 μm or more and 1 mm or less. Since the sensor height I1 is 5 μm or more, the variation in the measurement of the measuring instrument 10I can be suppressed as compared to a case where the sensor height I1 is less than 5 μm. Since the sensor height I1 is 1 mm or less, a possibility that an excess pressure is applied to the measurement target when the sensor unit 3C comes into contact with the measurement target can be reduced, as compared to a case where the sensor height I1 is larger than 1 mm. In a case where the excess pressure is applied to the measurement target when the sensor unit 3C comes into contact with the measurement target, there is a possibility that pain is felt when the measurement target is a person.

### 1.10.2 Effects and the Like

In the measuring instrument 10I described above, the sensor unit 3C has the surface 300 exposed from the head portion 21C. The head portion 21C has the frame-shaped region 200 surrounding the surface 300 of the sensor unit 3C. At least a part of the surface 300 of the sensor unit 3C is recessed with respect to the frame-shaped region 200 of the head portion 21C. As compared to a case where at least a part of the surface 300 of the sensor unit 3C neither protrudes nor is recessed with respect to the frame-shaped region 200 of the head portion 21C, since the close contact of the sensor unit 3C to the measurement target is improved, the measurement is stabilized, and this configuration can improve the detection accuracy of the electrostatic capacity.

In the measuring instrument 10I, the entire surface 300 of the sensor unit 3C is recessed from the frame-shaped region 200 of the head portion 21C. Since the close contact of the sensor unit 3C to the measurement target is further improved, the measurement is stabilized, and thus, this configuration can further improve the detection accuracy of the electrostatic capacity.

In the measuring instrument 10I, a distance (sensor height I1) between the surface 300 of the sensor unit 3C and the predetermined plane including the frame-shaped region 200 of the head portion 21C is 5 μm or more and 1 mm or less. This configuration can improve the detection accuracy of the electrostatic capacity while reducing a possibility that an excess pressure is applied to the measurement target when the sensor unit 3C comes into contact with the measurement target.

### 1.11 Eleventh Embodiment

### 1.11.1 Configuration

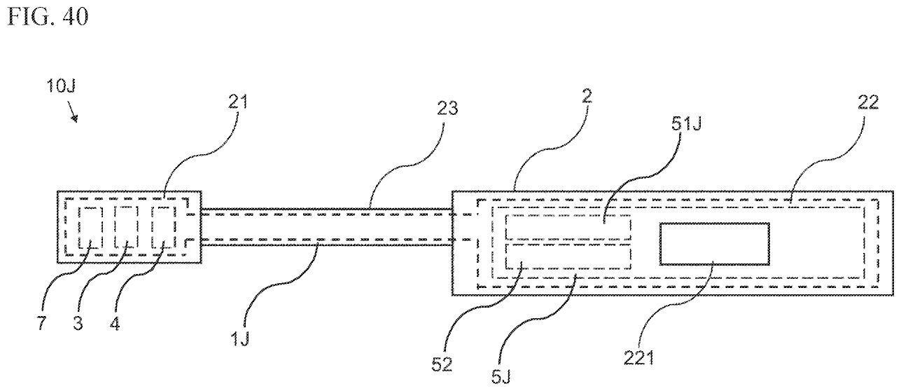

FIG. 40 is a schematic diagram of a configuration example of a measuring instrument 10J according to an eleventh embodiment. The measuring instrument 10J in FIG. 40 is an electrostatic capacity type moisture measuring instrument. The measuring instrument 10J includes an electrostatic capacity sensor 1J and the handheld housing 2.

The electrostatic capacity sensor 1J determines the moisture content of the measurement target based on the electrostatic capacity. The electrostatic capacity sensor 1J includes the sensor unit 3, the electrostatic capacity detection circuit 4, a processing circuit 5J, and a load detection circuit 7. In the present embodiment, the sensor unit 3, the electrostatic capacity detection circuit 4, and the load detection circuit 7 are positioned in the head portion 21 of the handheld housing 2. In the present embodiment, the processing circuit 5J is positioned in the grip portion 22 of the handheld housing 2.

The load detection circuit 7 detects a load received by the sensor unit 3 from the measurement target. The load detection circuit 7 may detect the load itself received by the sensor unit 3 from the measurement target or a physical quantity correlated with the load. The load detection circuit 7 may include, for example, a pressure sensor.

The processing circuit 5J includes a calculation circuit 51J and the input and output circuit 52.

The calculation circuit 51J is connected to the input and output circuit 52. In a case where an operation to start measuring the moisture content is performed by the input device of the input and output circuit 52, the calculation circuit 51J causes the electrostatic capacity detection circuit 4 to start the operation for detecting the electrostatic capacity. The calculation circuit 51J is configured to calculate the electrostatic capacity of the capacitor 30 based on the charging and discharging time of the capacitor 30 by the electrostatic capacity detection circuit 4. The calculation circuit 51J is configured to obtain the moisture content of the measurement target based on the electrostatic capacity of the capacitor 30. The calculation circuit 51J displays the moisture content of the measurement target by the output device of the input and output circuit 52.

Here, in a case where the contact between the sensor unit 3 and the measurement target is insufficient, the reliability of the calculated electrostatic capacity of the capacitor 30 may be low. When the reliability of the electrostatic capacity of the capacitor 30 is low, naturally, the reliability of the calculation result (the moisture content of the measurement target) based on the electrostatic capacity of the capacitor 30 is also reduced. From this perspective, in the present embodiment, the calculation circuit 51J is configured to determine whether or not to output the calculation result (the moisture content of the measurement target) based on the electrostatic capacity of the capacitor in accordance with the load received by the sensor unit 3 from the measurement target. More specifically, when the load detection circuit 7 acquires the load received by the sensor unit 3 from the measurement target, the calculation circuit 51J compares the load received by the sensor unit 3 from the measurement target with a predetermined value. The calculation circuit 51J outputs the calculation result (the moisture content of the measurement target) based on the electrostatic capacity of the capacitor while the load received by the sensor unit 3 from the measurement target is equal to or larger than the predetermined value. The processing circuit 5J does not output the calculation result (the moisture content of the measurement target) based on the electrostatic capacity of the capacitor while the load received by the sensor unit 3 from the measurement target is less than the predetermined value. The predetermined value is, for example, 2.3 gf/mm².

As described above, the processing circuit 5J outputs the calculation result (the moisture content of the measurement target) based on the electrostatic capacity of the capacitor while the load received by the sensor unit 3 from the measurement target is equal to or larger than the predetermined value, and does not output the calculation result (the moisture content of the measurement target) based on the electrostatic capacity of the capacitor while the load received by the sensor unit 3 from the measurement target is less than the predetermined value. Accordingly, only in a case where the electrostatic capacity of the capacitor 30 is reliable, since the calculation result can be outputted, the detection accuracy of the electrostatic capacity can be improved.

In the present embodiment, the probe portion 23 is formed such that the position of the head portion 21 with respect to the grip portion 22 changes in accordance with the load received by the sensor unit 3 from the measurement target. The probe portion 23 is formed such that the head portion 21 is inclined to a front side with respect to a length direction of the grip portion 22 in a case where the load received by the sensor unit 3 from the measurement target is 0 (at the time of no load). In a case where the load received by the sensor unit 3 from the measurement target is the predetermined value, the probe portion 23 is formed such that the head portion 21 is in parallel with the length direction of the grip portion 22. As an example, the probe portion 23 can be made of a material having spring properties.

### 1.11.2 Effects and the Like

In the measuring instrument 10J described above, the processing circuit 5J outputs the calculation result based on the electrostatic capacity of the capacitor 30 while the load received by the sensor unit 3 from the measurement target is equal to or larger than the predetermined value, and does not output the calculation result based on the electrostatic capacity of the capacitor 30 while the load received by the sensor unit 3 from the measurement target is less than the predetermined value. Only in a case where the electrostatic capacity of the capacitor 30 is reliable, since the calculation result can be outputted, this configuration can improve the detection accuracy of the electrostatic capacity.

### 2. Modification Example

The embodiments of the present disclosure are not limited to the embodiments described above. The embodiments described above can be modified in various ways depending on the design and the like as long as the possible benefit of the present disclosure can be achieved. Modification examples of the embodiments described above are listed below. Modification examples to be described below can be applied in combination as appropriate.

In a modification example, the first to fourth switches S1 to S4 of the electrostatic capacity detection circuit 4 are not necessarily field effect transistors. The first to fourth switches S1 to S4 may be semiconductor switches or mechanical switches. In a case where the third switch S3 and the fourth switch S4 are not field effect transistors and do not have body diodes, Vf2 in Equation (8) is a lower limit value of the potential V2 of the second electrode 32 when the charge and discharge circuit 42 in a case where there is not the second auxiliary capacitor 44b is switched from the first state to the second state, and the magnitude of Vf2 is equal to the magnitude of the second threshold. Vf9 in Equation (9) is a lower limit value of the potential V1 of the first electrode 31 when the charge and discharge circuit 42 in a case where there is not the first auxiliary capacitor 44a is switched from the second state to the first state, and the magnitude of Vf1 is equal to the magnitude of the first threshold.

In the modification example, the structures of the sensor unit 3 and 3B are not particularly limited. The sensor units 3 and 3B may have a known structure in the related art. In the case of a moisture meter, the sensor unit 3 may be formed such that the first and second electrodes 31 and 32 form the capacitor 30 together with a part of the measurement target by bringing the first and second electrodes 31 and 32 into contact with the measurement target. In the case of an occlusal force meter, the sensor unit 3B includes the deformation portion 35B deformed by the applied pressure, and may be formed such that the first and second electrodes 31B and 32B form the capacitor 30B together with the deformation portion 35B.

In the embodiment, the electrostatic capacity is calculated based on the period T, but the present disclosure is not limited thereto, and may be measured by impedance measurement or the like. The electrostatic capacity is not limited to a total capacitance, and only the auxiliary capacitor may be measured and calculated.

In addition, in order to maintain the conditions at the time of startup of the measuring instrument to be constant, the operation to discharge the charges of the capacitor may be performed before startup and then the measurement may be started. The present disclosure is not limited thereto, and the measurement may be performed after the capacitor is fully charged. There is a possibility that the variation in the charges stored in the capacitor at the time of startup adversely affects the measurement result. Then, the charges at the time of startup are set under a constant condition, and thus, it is possible to reduce an adverse effect and improve the measurement accuracy.

In the fourth and sixth embodiments, the protruding region 341 and the recessed region 342 on the surface 340 of the protective layer 34C may be disposed regardless of the electrode portions 311 and 321 on the first surface 33a of the sensor substrate 33. In the fifth and seventh embodiments, the protruding region 341 and the recessed region 342 of the surface 340 of the protective layer 34D may be disposed regardless of the electrode portions 311 and 321 of the first surface 33a of the sensor substrate 33.

In a modification example, the frame-shaped region 200 of the head portion 21C may have a rough surface having an uneven shape or irregular unevenness. In this case, the surface 300 of the sensor unit 3C may be flat. That is, the contact region 100 may have a rough surface having an uneven shape or irregular unevenness or both the uneven shape and the rough surface, and the surface 300 of the sensor unit 3C do not have a rough surface having an uneven shape or irregular unevenness or both the uneven shape and the rough surface. Note that, when the measuring instrument 10C is used, the head portion 21C may be covered with a protective resin film. It is desirable that the rough surface having the uneven shape or irregular unevenness has such a dimensional shape that the measuring instrument 10C functions even from the above of such a resin film.

In the ninth and tenth embodiments, any one of the sensor units 3, and 3D to 3G may be employed instead of the sensor unit 3C. In the ninth embodiment, the surface 300 of the sensor unit 3C may have a curved surface (protruding surface). As a result, a part, but not all, of the surface 300 of the sensor unit 3C may protrude from the frame-shaped region 200 of the head portion 21C. In the tenth embodiment, the surface 300 of the sensor unit 3C may have a curved surface (recessed surface). As a result, a part, but not all, of the surface 300 of the sensor unit 3C may be recessed from the frame-shaped region 200 of the head portion 21C. The shape of the surface 300 of the sensor unit 3C can be appropriately set depending on how much all or a part of the surface 300 of the sensor unit 3C protrudes or is recessed with respect to the frame-shaped region 200 of the head portion 21C.

In the eleventh embodiment, any one of the sensor units 3B to 3G may be employed instead of the sensor unit 3.

### 3. Aspects

As is clear from the embodiments and the modification examples described above, the present disclosure includes the following aspects. Hereinafter, the reference numerals are attached with parentheses only for clarifying the correspondence relationship with the embodiments. Note that, in consideration of the readability of the text, the description of the reference numerals in parentheses may be omitted from the second time onwards.

A first aspect is an electrostatic capacity sensor (1; 1A; 1B to 1G; 1J), and includes a sensor unit (3; 3B) having a first electrode (31) and a second electrode (32) constituting a capacitor (30), and an electrostatic capacity detection circuit (4; 4A) connected to the sensor unit (3; 3B to 3G). The electrostatic capacity detection circuit (4; 4A) includes a charge and discharge circuit (42) that is connected to the first electrode (31) and the second electrode (32) to charge and discharge the capacitor (30), a control circuit (43) that controls the charge and discharge circuit (42) such that the capacitor (30) repeats charge and discharge, and an auxiliary capacity circuit (44; 44A) that has at least one of a first auxiliary capacitor (44a) connected to the first electrode (31) in parallel with the capacitor (30) and a second auxiliary capacitor (44b) connected to the second electrode (32) in parallel with the capacitor (30). This aspect can reduce the influence of the stray capacitance on the detection of the electrostatic capacity.

A second aspect is the electrostatic capacity sensor (1; 1B to 1G; 1J) based on the first aspect. In the second aspect, the auxiliary capacity circuit (44) includes the first auxiliary capacitor (44a) and the second auxiliary capacitor (44b). This aspect can reduce the influence of the stray capacitance on the detection of the electrostatic capacity.

A third aspect is the electrostatic capacity sensor (1; 1B to 1G; 1J) based on the second aspect. In the third aspect, an electrostatic capacity of the first auxiliary capacitor (44a) and an electrostatic capacity of the second auxiliary capacitor (44b) are equal. This aspect can reduce the influence of the stray capacitance on the detection of the electrostatic capacity.

A fourth aspect is the electrostatic capacity sensor (1; 1B to 1G; 1J) based on the second aspect. In the fourth aspect, an electrostatic capacity of the first auxiliary capacitor (44a) and an electrostatic capacity of the second auxiliary capacitor (44b) are different. This aspect can reduce the influence of the stray capacitance on the detection of the electrostatic capacity.

A fifth aspect is the electrostatic capacity sensor (1; 1A; 1B to 1G; 1J) based on any one of the first to fourth aspects. In the fifth aspect, the charge and discharge circuit (42) is configured to be complementarily switchable between a first state where a constant output current is supplied to the first electrode (31) and a second state where a constant output current is supplied to the second electrode (32). The control circuit (43) is configured to switch the charge and discharge circuit (42) from the first state to the second state when a potential of the first electrode (31) reaches a first threshold in a case where the charge and discharge circuit (42) is in the first state. The control circuit (43) is configured to switch the charge and discharge circuit (42) from the second state to the first state when a potential of the second electrode (32) reaches a second threshold in a case where the charge and discharge circuit (42) is in the second state. This aspect can simplify the configuration of the electrostatic capacity detection circuit.

A sixth aspect is the electrostatic capacity sensor (1; 1A; 1B to 1G; 1J) based on the fifth aspect. In the sixth aspect, the first threshold and the second threshold are equal. This aspect can simplify the configuration of the electrostatic capacity detection circuit.

A seventh aspect is the electrostatic capacity sensor (1; 1A; 1B to 1G; 1J) based on the fifth or sixth aspect. In the seventh aspect, the charge and discharge circuit (42) is connected between a power supply terminal (41a) connected to a power supply (Iin) and a reference potential terminal (41b) connected to a reference potential (Vg), and includes a first switch (S1), a second switch (S2), a third switch (S3), and a fourth switch (S4). The first switch (S1) and the third switch (S3) constitute a series circuit. The series circuit of the first switch (S1) and the third switch (S3) is present between the power supply terminal (41a) and the reference potential terminal (41b) such that the first switch (S1) is connected to the power supply terminal (41a) and the third switch (S3) is connected to the reference potential terminal (41b). A connection point of the first switch (S1) and the third switch (S3) is connected to the first electrode (31). The second switch (S2) and the fourth switch (S4) constitute a series circuit. The series circuit of the second switch (S2) and the fourth switch (S4) is present between the power supply terminal (41a) and the reference potential terminal (41b) such that the second switch (S2) is connected to the power supply terminal (41a) and the fourth switch (S4) is connected to the reference potential terminal (41b), and is connected in parallel with the series circuit of the first switch (S1) and the third switch (S3). A connection point of the second switch (S2) and the fourth switch (S4) is connected to the second electrode (32). In the first state, the first and fourth switches (S4) are turned on, and the second and third switches (S3) are turned off. In the second state, the first and fourth switches (S4) are turned off, and the second and third switches (S3) are turned on. This aspect can simplify the configuration of the electrostatic capacity detection circuit.

An eighth aspect is the electrostatic capacity sensor (1; 1A; 1B to 1G; 1J) based on the seventh aspect. In the eighth aspect, a first end of the first auxiliary capacitor (44a) is connected to the first electrode (31) and a second end of the first auxiliary capacitor (44a) is connected to the reference potential terminal (41b) such that the first auxiliary capacitor (44a) is in parallel with the third switch (S3). A first end of the second auxiliary capacitor (44b) is connected to the second electrode (32) and a second end of the second auxiliary capacitor (44b) is connected to the reference potential terminal (41b) such that the second auxiliary capacitor (44b) is in parallel with the fourth switch (S4). This aspect can reduce the influence of the stray capacitance on the detection of the electrostatic capacity.

A ninth aspect is the electrostatic capacity sensor (1; 1B to 1G; 1J) based on the eighth aspect. In the ninth aspect, the electrostatic capacity detection circuit (4) satisfies the following equations.

$$\begin{matrix}[ Math . 19 ] & \\ \frac{Ce}{Ce + Cg 1} \cdot \vert Vth 2 \vert \leq \vert Vf 2 \vert & \end{matrix}$$

$$\frac{Ce}{Ce + Cg 2} \cdot \vert Vth 1 \vert \leq \vert Vf 1 \vert$$

Ce is an electrostatic capacity of the capacitor (30). Cg1 is an electrostatic capacity of the first auxiliary capacitor (44a). Cg2 is an electrostatic capacity of the second auxiliary capacitor (44b). Vth1 is the first threshold. Vth2 is the second threshold. Vf1 is a lower limit value of the potential of the first electrode (31) when the charge and discharge circuit (42) in a case where there is not the first auxiliary capacitor (44a) is switched from the second state to the first state. Vf2 is a lower limit value of the potential of the second electrode (32) when the charge and discharge circuit (42) in a case where there is not the second auxiliary capacitor (44b) is switched from the first state to the second state. This aspect can reduce the influence of the stray capacitance on the detection of the electrostatic capacity.

A tenth aspect is the electrostatic capacity sensor (1; 1B to 1G; 1J) based on the ninth aspect. In the tenth aspect, Vf1=Vf2 is satisfied. This aspect can simplify the configuration of the electrostatic capacity detection circuit.

An eleventh aspect is the electrostatic capacity sensor (1; 1B to 1G; 1J) based on the ninth or tenth aspect. In the eleventh aspect, Vf1<0 and Vf2<0 are satisfied. In the aspect, the amount of change in the electrostatic capacity can be increased, and the detection accuracy of the electrostatic capacity can be improved.

A twelfth aspect is the electrostatic capacity sensor (1; 1B to 1G; 1J) based on any one of the ninth to eleventh aspects. In the twelfth aspect, each of the third switch (S3) and the fourth switch (S4) is a field effect transistor. Vf1 is determined by a threshold voltage of a body diode of the third switch (S3). Vf2 is determined by a threshold voltage of a body diode of the fourth switch (S4). In this aspect, it is possible to reduce a size of the electrostatic capacity detection circuit and increase a speed of switching between the first state and the second state.

A thirteenth aspect is the electrostatic capacity sensor (1; 1A; 1B to 1G; 1J) based on any one of the first to twelfth aspects. In the thirteenth aspect, the sensor unit (3; 3B to 3G) includes a sensor substrate (33) on which the first electrode (31) and the second electrode (32) are disposed. The charge and discharge circuit (42) is disposed on a circuit substrate (4a) different from the sensor substrate (33). The auxiliary capacity circuit (44; 44A) is disposed between the sensor substrate (33) and the circuit substrate (4a) and at a position closer to the circuit substrate (4a) than the sensor substrate (33). In this aspect, the influence of the stray capacitance from the first electrode and the second electrode of the sensor unit can be reduced.

A fourteenth aspect is the electrostatic capacity sensor (1; 1A; 1B to 1G; 1J) based on any one of the first to thirteenth aspects. In the fourteenth aspect, the electrostatic capacity sensor (1; 1A; 1B to 1G; 1J) further includes a processing circuit (5; 5B; 5J) that calculates an electrostatic capacity of the capacitor (30) based on a charging and discharging time of the capacitor (30) by the electrostatic capacity detection circuit (4; 4A). This aspect can reduce the influence of the stray capacitance on the detection of the electrostatic capacity.

A fifteenth aspect is a measuring instrument (10; 10B; to 10J), and includes the electrostatic capacity sensor (1; 1A: 1B to 1G; 1J) based on any one of the first to thirteenth aspects and a handheld housing (2; 2B; 2C) that accommodates the electrostatic capacity sensor (1; 1A; 1B to 1G; 1J). This aspect can reduce the influence of the stray capacitance on the detection of the electrostatic capacity.

A sixteenth aspect is the measuring instrument (10; 10B to 10J) based on the fifteenth aspect. In the sixteenth aspect, the electrostatic capacity sensor (1; 1A; 1B to 1G; 1J) further includes a processing circuit (5; 5B; 5J) that calculates an electrostatic capacity of the capacitor (30) based on a charging and discharging time of the capacitor (30) by the electrostatic capacity detection circuit (4; 4A). This aspect can reduce the influence of the stray capacitance on the detection of the electrostatic capacity.

A seventeenth aspect is the measuring instrument (10; 10B to 10J) based on the sixteenth aspect. In the seventeenth aspect, the handheld housing (2; 2B; 2C) includes a head portion (21; 21B; 21C) that is disposed at a first end of the handheld housing (2; 2B; 2C) to come into contact with a measurement target, a grip portion (22; 22B) that is disposed at a second end of the handheld housing (2; 2B; 2C) and is gripped with hand, a probe portion (23) that couples the head portion (21; 21B; 21C) and the grip portion (22; 22B). The sensor unit (3; 3B to 3G) is positioned in the head portion (21; 21B; 21C). The electrostatic capacity detection circuit (4) is positioned in the head portion (21; 21B; 21C) or the probe portion (23). The processing circuit (5; 5B; 5J) is positioned in the grip portion (22; 22B). According to this aspect, the influence of the stray capacitance generated in the grip portion can be reduced.

An eighteenth aspect is the measuring instrument (10; 10B to 10J) based on the seventeenth aspect. In the eighteenth aspect, the grip portion (22; 22B) has a conductive portion (221; 221B) that is exposed on a surface of the grip portion (22; 22B). The conductive portion (221; 221B) is connected to a reference potential (Vg) of the processing circuit (5; 5B; 5J). In this aspect, it is possible to reduce the variation in the influence of the stray capacitance on the side of the person who has the measuring instrument.

A nineteenth aspect is the measuring instrument (10C) based on any one of the sixteenth to eighteenth aspects. In the nineteenth aspect, the sensor unit (3C; 3D; 3E; 3F) has a surface (300) that is exposed from the head portion (21C). The surface (300) of the sensor unit (3C; 3D; 3E; 3F) has an uneven shape. As compared to a case where the surface of the sensor unit is flat, this aspect can increase the specific surface area of the sensor unit and the friction coefficient (mainly, static friction coefficient) of the surface of the sensor unit with respect to the measurement target and can improve the detection accuracy of the electrostatic capacity.

A twentieth aspect is the measuring instrument (10H; 10I) based on the seventeenth aspect. In the twentieth aspect, the sensor unit (3C) has a surface (300) that is exposed from the head portion (21C). The head portion (21C) has a frame-shaped region (200) surrounding a surface (300) of the sensor unit (3C). At least a part of the surface (300) of the sensor unit (3C) protrudes or is recessed with respect to the frame-shaped region (200) of the head portion (21C). As compared to a case where at least a part of the surface of the sensor unit neither protrudes nor is recessed with respect to the frame-shaped region of the head portion, since the close contact of the sensor unit to the measurement target is improved, the measurement is stabilized, and this aspect can improve the detection accuracy of the electrostatic capacity. Since the static electricity charged in the sensor unit 3C is effectively discharged, the variation in the measurement result due to the charging of the sensor unit 3C is suppressed, and thus, this configuration can improve the detection accuracy of the electrostatic capacity.

A twenty-first aspect is the measuring instrument (10H) based on the twentieth aspect. In the twenty-first aspect, the entire surface (300) of the sensor unit (3C) protrudes from the frame-shaped region (200) of the head portion (21C). Since the close contact of the sensor unit to the measurement target is further improved, the measurement is stabilized, and thus, this aspect can further improve the detection accuracy of the electrostatic capacity. Since the static electricity charged in the sensor unit 3C is more effectively discharged, the variation in the measurement result due to the charging of the sensor unit 3C is suppressed, and thus, this configuration can improve the detection accuracy of the electrostatic capacity.

A twenty-second aspect is the measuring instrument (10H; 10I) based on the twentieth aspect. In the twenty-second aspect, a distance between the surface (300) of the sensor unit (3C) and the predetermined plane is 5 μm or more and 1 mm or less. This aspect can improve the detection accuracy of the electrostatic capacity while reducing a possibility that an excess pressure is applied to the measurement target when the sensor unit comes into contact with the measurement target.

A twenty-third aspect is the measuring instrument (10J) based on the seventeenth aspect. In the twenty-third aspect, the processing circuit (5J) outputs a calculation result based on the electrostatic capacity of the capacitor (30) while the load received by the sensor unit (3) from the measurement target is equal to or more than a predetermined value, and does not output the calculation result based on the electrostatic capacity of the capacitor (30) while the load received by the sensor unit (3) from the measurement target is less than the predetermined value. Only in a case where the electrostatic capacity of the capacitor is reliable, since the calculation result can be outputted, the detection accuracy of the electrostatic capacity can be improved.

A twenty-fourth aspect is the measuring instrument (10; 10C to 10J) based on any one of the sixteenth to twenty-third aspects. In the twenty-fourth aspect, the sensor unit (3; 3C to 3G) is formed such that the first and second electrodes (31, 32) form the capacitor (30) together with a part of a measurement target by bringing the first and second electrodes (31, 32) into contact with the measurement target. The processing circuit (5; 5J) is configured to obtain the moisture content of the measurement target based on the electrostatic capacity of the capacitor (30). This aspect enables the measurement of the moisture content of the measurement target.

A twenty-fifth aspect is the measuring instrument (10; 10C to 10J) based on the twenty-fourth aspect. In the twenty-fifth aspect, the measurement target is an organism. This aspect enables the measurement of the moisture content of the organism.

A twenty-sixth aspect is the measuring instrument (10; 10C to 10J) based on the twenty-fourth or twenty-fifth aspect. In the twenty-sixth aspect, the measurement target is an oral cavity of an organism. This aspect enables the measurement of the moisture content in the oral cavity of the organism.

A twenty-seventh aspect is the measuring instrument (10B) based on the twenty-sixth aspect. In the twenty-seventh aspect, the sensor unit (3B) includes a deformation portion (35B) that is deformed by an applied pressure. The sensor unit (3B) is formed such that the first and second electrodes (31B, 32B) form the capacitor (30B) together with the deformation portion (35B). The processing circuit (5) is configured to obtain the pressure based on the electrostatic capacity of the capacitor (30B). This aspect enables the measurement of the pressure. In particular, the pressure may be applied to the deformation portion (35B) by a person biting with the upper and lower jaw teeth. In this case, it is possible to measure the occlusal force of the upper and lower jaw teeth of a person.

The second to fourteenth aspects and the sixteenth to twenty-seventh aspects are not essential.

The present disclosure is applicable to an electrostatic capacity detection circuit, an electrostatic capacity sensor, and a measuring instrument. Specifically, the present disclosure is applicable to an electrostatic capacity detection circuit for detecting an electrostatic capacity based on charge and discharge of a capacitor, an electrostatic capacity sensor including the electrostatic capacity detection circuit, and a measuring instrument including the electrostatic capacity sensor.
10, 10B to 10J measuring instrument
1, 1A, 1B to 1G, 1J electrostatic capacity sensor
2, 2B, 2C handheld housing
21, 21B, 21C head portion
22, 22B grip portion
221, 221B conductive portion
23 probe portion
3, 3B to 3G sensor unit
30, 30B capacitor
31B first electrode
32B second electrode
33 sensor substrate
35B deformation portion
4, 4A electrostatic capacity detection circuit
41a power supply terminal
41b reference potential terminal
42 charge and discharge circuit
S1 first switch
S2 second switch
S3 third switch
S4 fourth switch
43 control circuit
44, 44A auxiliary capacity circuit
44a first auxiliary capacitor
44b second auxiliary capacitor
4a circuit substrate
5, 5B, 5J processing circuit
Iin power supply
Vg reference potential

1. An electrostatic capacity sensor comprising:
a sensor comprising a first electrode and a second electrode constituting a capacitor; and
an electrostatic capacity detection circuit that is connected to the sensor,
wherein the electrostatic capacity detection circuit comprises:
a charge and discharge circuit that is connected to the first electrode and the second electrode, and that is configured to charge and discharge the capacitor,
a control circuit configured to control the charge and discharge circuit such that the capacitor repeatedly charges and discharges, and
an auxiliary capacity circuit comprising a first auxiliary capacitor connected to the first electrode in parallel with the capacitor, or a second auxiliary capacitor connected to the second electrode in parallel with the capacitor.

2. The electrostatic capacity sensor according to claim 1, wherein the auxiliary capacity circuit comprises the first auxiliary capacitor and the second auxiliary capacitor.

3. The electrostatic capacity sensor according to claim 2, wherein an electrostatic capacity of the first auxiliary capacitor is equal to an electrostatic capacity of the second auxiliary capacitor.

4. The electrostatic capacity sensor according to claim 2, wherein an electrostatic capacity of the first auxiliary capacitor is different than an electrostatic capacity of the second auxiliary capacitor.

5. The electrostatic capacity sensor according to claim 1,
wherein the charge and discharge circuit is configured to complementarily switch between a first state where a constant output current is supplied to the first electrode and a second state where a constant output current is supplied to the second electrode, and
wherein the control circuit is configured to switch the charge and discharge circuit from the first state to the second state when a potential of the first electrode reaches a first threshold and the charge and discharge circuit is in the first state, and to switch the charge and discharge circuit from the second state to the first state when a potential of the second electrode reaches a second threshold value and the charge and discharge circuit is in the second state.

6. The electrostatic capacity sensor according to claim 5, wherein the first threshold is equal to the second threshold.

7. The electrostatic capacity sensor according to claim 5,
wherein the charge and discharge circuit is connected between a power supply terminal connected to a power supply and a reference potential terminal connected to a reference potential, and comprises a first switch, a second switch, a third switch, and a fourth switch,
wherein the first switch and the third switch constitute a first series circuit,
wherein the first series circuit is connected between the power supply terminal and the reference potential terminal such that the first switch is connected to the power supply terminal and the third switch is connected to the reference potential terminal,
wherein a connection point of the first switch and the third switch is connected to the first electrode,
wherein the second switch and the fourth switch constitute a second series circuit,
wherein the second series circuit is connected between the power supply terminal and the reference potential terminal such that the second switch is connected to the power supply terminal and the fourth switch is connected to the reference potential terminal, and is connected to the first series circuit,
wherein a connection point of the second switch and the fourth switch is connected to the second electrode,
wherein in the first state, the first and fourth switches are ON, and the second and third switches are OFF, and
wherein in the second state, the first and fourth switches are OFF, and the second and third switches are ON.

8. The electrostatic capacity sensor according to claim 7,
wherein a first end of the first auxiliary capacitor is connected to the first electrode and a second end of the first auxiliary capacitor is connected to the reference potential terminal such that the first auxiliary capacitor is in parallel with the third switch, and
a first end of the second auxiliary capacitor is connected to the second electrode and a second end of the second auxiliary capacitor is connected to the reference potential terminal such that the second auxiliary capacitor is in parallel with the fourth switch.

9. The electrostatic capacity sensor according to claim 8, wherein in the electrostatic capacity detection circuit:
$$\frac{Ce}{Ce + Cg 1} \cdot \vert Vth 2 \vert \leq \vert Vf 2 \vert$$
$$and$$
$$\frac{Ce}{Ce + Cg 2} \cdot \vert Vth 1 \vert \leq \vert Vf 1 \vert$$
and wherein:
Ce is an electrostatic capacity of the capacitor,
Cg1 is an electrostatic capacity of the first auxiliary capacitor,
Cg2 is an electrostatic capacity of the second auxiliary capacitor,
Vth1 is the first threshold,
Vth2 is the second threshold,
Vf1 is a lower limit value of the potential of the first electrode when the charge and discharge circuit is switched from the second state to the first state and the auxiliary capacity circuit does not comprise the first auxiliary capacitor, and
Vf2 is a lower limit value of the potential of the second electrode when the charge and discharge circuit is switched from the first state to the second state and the auxiliary capacity circuit does not comprise the second auxiliary capacitor.

10. The electrostatic capacity sensor according to claim 9, wherein Vf1=Vf2.

11. The electrostatic capacity sensor according to claim 9, wherein Vf1<0 and Vf2<0.

12. The electrostatic capacity sensor according to claim 9,
wherein each of the third switch and the fourth switch is a field effect transistor,
Vf1 is based on a threshold voltage of a body diode of the third switch, and
Vf2 is based on a threshold voltage of a body diode of the fourth switch.

13. The electrostatic capacity sensor according to claim 1,
wherein the sensor comprises a sensor substrate on which the first electrode and the second electrode are disposed,
wherein the charge and discharge circuit is disposed on a circuit substrate different from the sensor substrate, and
wherein the auxiliary capacity circuit is disposed between the sensor substrate and the circuit substrate and at a position closer to the circuit substrate than the sensor substrate.

14. The electrostatic capacity sensor according to claim 1, further comprising:
a processing circuit configured to calculate an electrostatic capacity of the capacitor based on a time of charging and discharging of the capacitor by the electrostatic capacity detection circuit.

15. A measuring instrument comprising:
the electrostatic capacity sensor according to claim 1;
a handheld housing that accommodates the electrostatic capacity sensor; and
a processing circuit configured to calculate an electrostatic capacity of the capacitor based on a charging and discharging time of the capacitor by the electrostatic capacity detection circuit.

16. The measuring instrument according to claim 15,
wherein the handheld housing comprises:
a head portion that is disposed at a first end of the handheld housing and that contacts a measurement target,
a grip portion that is disposed at a second end of the handheld housing and that is gripped with hand, and
a probe portion that couples the head portion and the grip portion,
wherein the sensor is in the head portion,
wherein the electrostatic capacity detection circuit is in the head portion or the probe portion,
wherein the processing circuit is in the grip portion,
wherein the grip portion has a conductive portion that is exposed on a surface of the grip portion,
wherein the conductive portion is connected to a reference potential of the processing circuit, and
wherein the sensor has a surface that is exposed from the head portion.

17. The measuring instrument according to claim 16, wherein a distance between the surface of the sensor and a predetermined plane including a region of the head portion surrounding the surface of the sensor is 5 μm or more and 1 mm or less.

18. The measuring instrument according to claim 15,
wherein the first and second electrodes form the capacitor together with a part of a measurement target when the first and second electrodes are in contact with the measurement target, and
wherein the processing circuit is configured to obtain a moisture content of the measurement target based on the electrostatic capacity of the capacitor.

19. The measuring instrument according to claim 18, wherein the measurement target is an oral cavity.

20. The measuring instrument according to claim 19,
wherein the sensor has a deformation portion that is configured to deform when pressure is applied to the deformation portion,
wherein the the first and second electrodes form the capacitor together with the deformation portion, and
wherein the processing circuit is configured to measure the pressure based on the electrostatic capacity of the capacitor.
